(19) United States
(12) Patent Application Publication
(10) Pub. No.: US 2002/0035137 A1
(43) Pub. Date: Mar. 21, 2002

(54) Amino (oxo) acetic acid protein tyrosine phosphatase inhibitors

(76) Inventors: Gang Liu, Gurnee, IL (US); Bruce G. Szczepankiewicz, Lindenhurst, IL (US); Zhonghua Pei, Libertyville, IL (US); Zhili Xin, Lake Bluff, IL (US); Thorsten K. Oost, Pleasant Prairie, WI (US); David A. Janowick, Beach Park, IL (US)

Correspondence Address: ABBOTT LABORATORIES, D-0377 / AP6D-2, 100 Abbott Park Road, Abbott Park, IL, 60064-6050, US

(21) Appl. No.: 09/918,928

(22) Filed: Jul. 31, 2001

Related U.S. Application Data
(60) Provisional application No. 60/228,651, filed on Aug. 29, 2000.

(51) Int. Cl. A61K 31/66; C07D 417/02; C07D 413/02; C 07D 4 3/02; A61K 31/506; A61K 31/501; A61K 31/497; A61K 31/4439

(52) U.S. Cl. 514/360; 514/362; 514/364; 514/369; 514/372; 514/376; 514/380; 514/381; 514/386; 514/114; 514/252.05; 514/255.05; 514/256; 514/340; 544/238; 544/333; 544/322; 544/405; 546/268.1; 546/269.1; 546/274.4; 546/271.1; 546/272.1; 546/276.1; 548/122; 548/134; 548/132; 548/187; 548/243; 548/253; 548/319.1; 548/366.7

(57) ABSTRACT

Compounds of formula (I)

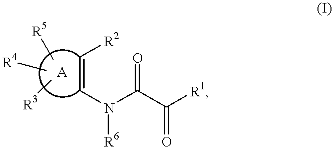

or therapeutically acceptable salts thereof, are protein tyrosine kinase PTP1B inhibitors. Preparation of the compounds, compositions containing the compounds, and treatment of diseases using the compounds are disclosed.

This application claims priority to the provisional application Serial No. 60/228,651 filed on Aug. 29, 2000.

### TECHNICAL FIELD

The instant invention is directed to compounds useful for inhibiting protein tyrosine phosphatase PTP1B, preparation of the compounds, compositions containing the compounds, and treatment of diseases using the compounds.

### BACKGROUND OF THE INVENTION

PTP1B belongs to a family of protein tyrosine phosphatases involved in the regulation of the cellular signaling mechanisms which are implicated in metabolism, growth, proliferation, and differentiation (Science 1991, 253, 401-406). Overexpression or altered activity of tyrosine phosphatase PTP1B can contribute to the progression of various diseases (Ann. Rev. Biochem. 1985, 54, 897-930). Two independent studies have indicated that PTP 1B knock-out mice have increased glucose tolerance, increased insulin sensitivity and decreased weight gain on a high fat diet. Furthermore, there is evidence which suggests inhibition of protein tyrosine phosphatase PTP1B is therapeutically beneficial for the treatment of diseases such as type I and II diabetes, obesity, autoimmune disease, acute and chronic inflammation, osteoporosis and various forms of cancer (J. Natl. Cancer Inst. 1994, 86, 372-378; Mol. Cell. Biol. 1994, 14, 6674-6682; The EMBO J. 1993, 12, 1937-1946; J. Biol. Chem. 1994, 269, 30659-30667; and Biochemical Pharmacology 1997, 54, 703-711).

Because of the important role played by unregulated protein tyrosine phosphatase PTP1B in these diseases, agents which inhibit the enzyme have been the subject of active current research for their clinical potential. Reference is made to WO 99/46236, WO 99/46237, WO 99/46267 and WO 99/46268; and although each teaches certain heteroaryl and aryl amino(oxo)acetic acid protein tyrosine phosphatase PTP1B inhibitors, the potency of these compounds decreases drastically as pH levels increase from 5.5 (J. Biol. Chem. 2000, 275, 10300-10307; and J. Biol. Chem. 2000, 275, 7101-7108). The instant invention provides PTP1B inhibitors which demonstrate potency at physiological pH levels, making them more suitable for drug development.

### SUMMARY OF THE INVENTION

In the principle embodiment of the instant invention, therefore, is provided a protein tyrosine phosphatase PTP1B inhibitor of formula (I)

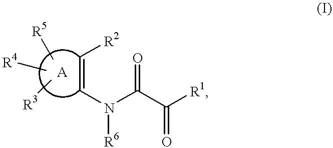

or a therapeutically acceptable salt thereof, wherein

A is selected from the group consisting of aryl, heteroaryl, and heterocycloalkyl;

R¹ is selected from the group consisting of alkoxy, alkyl, amino, aminosulfonyl, aryl, arylalkyl, aryloxy, hydroxy, perfluoroalkoxy, and perfluoroalkyl;

R² is selected from the group consisting of alkoxy, alkoxycarbonyl, alkyl, amido, amino, carboxy, cyano, nitro, SO₃H, PO(OH)₂, CH₂PO(OH)₂, CHFPO(OH)₂, CF₂(PO(OH)₂, C(═NH)NH₂, and the following 5-membered heterocycles:

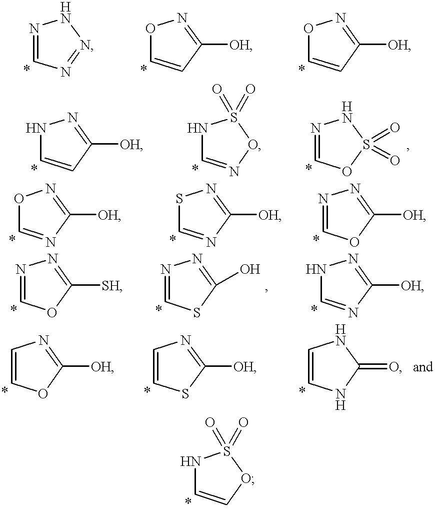

wherein * denotes the point of attachment to the parent molecular moeity;

R³, R⁴, and R⁵ are independently selected from the group consisting of hydrogen, alkoxy, alkyl, amido, amino, aminosulfonyl, arylcarbonylamino, cyano, halo, hydroxy, nitro, perfluoroalkoxy, and perfluoroalkyl; and

R⁶ is selected from the group consisting of alkyl, aryl, arylalkyl, cycloalkenyl, cycloalkenylalkyl, cycloalkyl, cycloalkylalkyl, heteroaryl, heteroarylalkyl, heterocycloalkyl, and (heterocycloalkyl)alkyl.

In another embodiment of the instant invention is provided a compound of formula

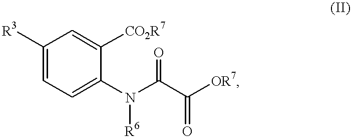

or a therapeutically acceptable salt thereof, wherein

R³ is selected from the group consisting of hydrogen, amido alkoxy, arylcarbonylamino, cyano and hydroxy;

R⁶ is selected from the group consisting of alkyl, aryl, arylalkyl, cycloalkenyl, cycloalkenylalkyl, cycloalkyl, cycloalkylalkyl, heteroaryl, heteroarylalkyl, heterocycloalkyl, and (heterocycloalkyl)alkyl; and

each R is independently selected from the group consisting of hydrogen and alkyl.

In a preferred embodiment of the compounds of formula (I), A is aryl.

In another preferred embodiment of the compounds of formula (I), A is aryl; and R¹ is hydroxy.

In another preferred embodiment of the compounds of formula (I), A is aryl; R¹ is hydroxy; and R² is carboxy.

In another preferred embodiment of the compounds of formula (1), A is aryl; R¹ is hydroxy; R² is carboxy; and R³, R⁴, and R⁵ are independently selected from the group consisting of hydrogen and alkoxy.

In another preferred embodiment of the compounds of formula (I), A is aryl; R¹ is hydroxy; R² is carboxy; R³, R⁴, and R⁵ are independently selected from the group consisting of hydrogen and alkoxy; and R⁶ is aryl, cycloalkyl, or heteroaryl.

In another embodiment of the instant invention is provided a method for inhibiting protein tyrosine phosphatase at physiological pH comprising administering a therapeutically effective amount of a compound of formula (I).

In another embodiment of the instant invention is provided a method for treating type II diabetes, obesity, impaired glucose tolerance and insulin resistance in a patient in recognized need thereof comprising administering to the patient a therapeutically effective amount of a compound of formula (I).

In another embodiment of the instant invention is provided a composition comprising a compound of formula (I) in combination with a therapeutically acceptable excipient.

In another embodiment of the instant invention is provided a method for inhibiting protein tyrosine phosphatase at physiological pH comprising administering a therapeutically effective amount of a compound of formula (II).

In another embodiment of the instant invention is provided a method for treating type II diabetes, obesity, impaired glucose tolerance and insulin resistance in a patient in recognized need thereof comprising administering to the patient a therapeutically effective amount of a compound of formula (II).

In another embodiment of the instant invention is provided a composition comprising a compound of formula (II) in combination with a therapeutically acceptable excipient.

### DETAILED DESCRIPTION OF THE INVENTION

The instant invention provides a series of compounds which inhibit protein tyrosine phosphatase PTP1B.

As used throughout the instant specification, the following terms have the meanings indicated:

The term “alkanoyl,” as used herein, represents an alkyl group attached to the parent molecular moiety through a carbonyl group.

The term “alkenyl,” as used herein, represents a monovalent straight or branched chain hydrocarbon radical having from two to six carbons and at least one carbon-carbon double bond.

The term “alkoxy,” as used herein, represents an alkyl group attached to the parent molecular moiety through an oxygen atom.

The term “alkoxycarbonyl,” as used herein, represents an alkoxy group attached to the parent molecular moiety through a carbonyl group.

The term “alkoxycarbonylalkenyl,” as used herein, represents an alkoxycarbonyl group attached to the parent molecular moiety through an alkenyl group.

The term “alkoxycarbonylalkyl,” as used herein, represents an alkoxycarbonyl group attached to the parent molecular moiety through an alkyl group.

The term “alkyl,” as used herein, represents a saturated, monovalent straight or branched chain hydrocarbon having from one to six carbons.

The term “alkylsufonyl,” as used herein, represents an alkyl group attached to the parent molecular moiety through a sulfonyl group.

The term “amido,” as used herein, represents an amino group attached to the parent molecular moiety through a carbonyl group.

The term “amidoalkenyl,” as used herein, represents an amido group attached to the parent molecular moiety through an alkenyl group.

The term “amidoalkyl,” as used herein, represents an amido group attached to the parent molecular moiety through an alkyl group. The alkyl part of the amidoalkyl can be optionally substituted with one or two substituents independently selected from hydroxy, thioalkoxy, R_{A}R_{B}N—, wherein R_{A} and R_{B} are independently selected from hydrogen, alkoxycarbonyl, alkyl, alkylsulfonyl, amido, aryl, arylalkyl, arylalkylcarbonyl, carboxyalkylcarbonyl, or a nitrogen protecting group.

The term “amino,” as used herein, represents —NR⁸R⁹, wherein R⁸ and R⁹ are independently selected from hydrogen, alkanoyl, alkenyl, alkoxycarbonyl, alkoxycarbonylalkyl, alkyl, alkylsulfonyl, aryl, arylalkyl, carboxyalkyl, cycloalkyl, cycloalkylalkyl, hydroxyalkyl, a nitrogen protecting group, phenylsulfonyl, and R_{A}R_{B}Ncarbonylalkyl, wherein R_{A} and R_{B} are previously defined or R⁸ and R⁹, together with the nitrogen atom to which they are attached, form a ring selected from the group consisting of morpholinyl, oxazinanyl, piperazinyl, piperidinyl, and pyrrolidinyl.

The term “aminoalkenyl,” as used herein, represents an amino group attached to the parent molecular moiety through an alkenyl group.

The term “aminoalkyl,” as used herein, represents an amino group attached to the parent molecular moiety through an alkyl group. The alkyl part of the aminoalkyl can be optionally substituted with one or two substituents independently selected from halogen and hydroxy.

The term “aminosulfonyl,” as used herein, represents an amino group attached to the parent molecular moiety through a sulfonyl group.

The term “aryl,” as used herein, represents dihydronaphthyl, indanyl, indenyl, naphthyl, phenyl, and tetrahydronaphthyl. Aryl groups having an unsaturated or partially saturated ring fused to an aromatic ring can be attached through the saturated or the unsaturated part of the group. The aryl groups of the instant invention can be optionally substituted with one, two, three, four, or five substituents independently selected from the group consisting of alkoxy, alkoxycarbonyl, alkoxycarbonylalkenyl, alkoxycarbonylalkyl, alkyl, alkylsufonyl, amido, amidoalkenyl, amidoalkyl, amino, aminoalkenyl, aminoalkyl, aminosulfonyl, carboxy, carboxyalkenyl, carboxyalkyl, cyano, halo, haloalkyl, heteroaryl, heteroarylalkyl, heterocycloalkyl, (heterocycloalkyl)alkyl, hydroxy, hydroxyalkyl, nitro, perfluoroalkoxy, perfluoroalkyl, phenyl, phenylalkoxy, phenylalkyl, phenylcarbonyl and thioalkoxy.

The term “arylalkyl,” as used herein, represents an aryl group attached to the parent molecular moiety through an alkyl group, wherein the alkyl part of the arylalkyl can be optionally substituted with amido and NR_{A}R_{B}, wherein R_{A} and R_{B} are previously defined.

The term “arylalkylcarbonyl” as used herein, represents an arylalkyl group attached to the parent molecular moiety through a carbonyl.

The term “aryloxy,” as used herein, represents an aryl group attached to the parent molecular moiety through an oxygen atom.

The term “carbonyl,” as used herein, represents —C(O)—.

The term “carboxy,” as used herein, represents —CO₂H.

The term “carboxyalkenyl,” as used herein, represents a carboxy group attached to the parent molecular moiety through an alkenyl group.

The term “carboxyalkyl,” as used herein, represents a carboxy group attached to the parent molecular moiety through an alkyl group.

The term“carboxyalkylcarbonyl,” as used herein, represents a carboxyalkyl group attached to the parent molecular moiety through a carbonyl group.

The term “cyano,” as used herein, represents —CN.

The term “cycloalkenyl,” as used herein, represents a monovalent cyclic or bicyclic hydrocarbon of four to twelve carbons having at least one carbon-carbon double bond.

The term “cycloalkenylalkyl,” as used herein, represents a cycloalkenyl group attached to the parent molecular moiety through an alkyl group.

The term “cycloalkyl,” as used herein, represents a monovalent saturated cyclic or bicyclic hydrocarbon group of three to twelve carbons. The cycloalkyl groups of the invention can be optionally substituted with one, two, three, or four substituents independently selected from the group consisting of alkanoyl, alkoxy, alkoxycarbonyl, alkyl, amido, carboxy, halo and hydroxy.

The term “cycloalkylalkyl,” as used herein, represents a cycloalkyl group attached to the parent molecular moiety through an alkyl group.

The term “halo,” represents to F, Cl, Br, or I.

The term “haloalkyl,” represents a halo group attached to the parent molecular moiety through an alkyl group.

The term “heteroaryl,” as used herein, represents cyclic, aromatic groups having five or six atoms, wherein at least one atom is selected from the group consisting of nitrogen, oxygen, and sulfur, and the remaining atoms are carbon. The five-membered rings have two double bonds, and the six-membered rings have three double bonds. Heteroaryls of the invention are exemplified by furanyl, thienyl, pyrrolyl, oxazolyl, thiazolyl, imidazolyl, isoxazolyl, isothiazolyl, oxadiazolyl, oxadiazolyl, triazolyl, thiadiazolyl, pyridyl, pyridazinyl, pyrimidinyl, pyrazinyl, pyrazolyl, triazinyl, and the like. The heteroaryl groups of the instant invention are connected to the parent molecular group through a carbon atom in the ring or, as exemplified by imidazole, indole, and pyrazole, through either a carbon atom or nitrogen atom in the ring. The heteroaryl groups of the invention can also be fused to a second ring selected from the group consisting of heteroaryl, heterocycloalkyl, and phenyl, in which case the heteroaryl group can be connected to the parent molecular group through either the heteroaryl part, the heterocycloalkyl part, or the phenyl part of the fused ring system. Heteroaryl groups of this type are exemplified by quinolinyl, isoquinolinyl, benzofuranyl, benzothiophenyl, benzoisoxazolyl, benzthiazolyl, benzooxazolyl, indolyl, thienopyrazinyl, thienylfuranyl, thienylpyridinyl, 2,3-dihydrothienofuranyl, and the like. The heteroaryl groups of this invention can be optionally substituted with one, two, or three substituents independently selected from the group consisting of alkoxy, alkoxycarbonyl, alkoxycarbonylalkenyl, alkoxycarbonylalkyl, alkyl, amido, amidoalkenyl, amidoalkyl, amino, aminoalkenyl, aminoalkyl, carboxy, carboxyalkenyl, carboxyalkyl, cyano, halo, haloalkyl, heterocycloalkyl, hydroxy, hydroxyalkyl, nitro, perfluoroalkoxy, perfluoroalkyl, phenyl, phenylalkoxy, phenylalkyl, and thioalkoxy.

The term “heteroarylalkyl,” as used herein, represents a heteroaryl group attached to the parent molecular moiety through an alkyl group.

The term “heterocycloalkyl,” as used herein, represents cyclic, non-aromatic, four-, five-, or six-membered groups containing at least one atom selected from the group consisting of oxygen, nitrogen, and sulfur. The four-membered rings have zero double bonds, the five-membered rings have zero or one double bonds, and the six-membered rings have zero, one, or two double bonds. Heterocycloalkyl groups of the invention are exemplified by dihydropyridinyl, imidazolinyl, morpholinyl, piperazinyl, pyrrolidinyl, pyrazolidinyl, tetrahydropyridinyl, piperidinyl, thiomorpholinyl, 1,3-dioxolanyl, 1,4-dioxanyl, 1,3-dioxanyl, and the like. The heterocycloalkyls of the instant invention can be attached to the parent molecular group through a carbon atom or nitrogen atom in the ring. The heterocycloalkyl groups of the invention can also be fused to a phenyl ring, in which case the heterocycloalkyl group can be connected to the parent molecular group through either the heterocycloalkyl part or the phenyl part of the fused ring system. Heterocycloalkyl groups of this type are exemplified by benzodioxolyl, indolinyl, tetrahydroquinolinyl, chromanyl, and the like. The heterocycloalkyl groups of this invention can be optionally substituted one, two, three, four or five substituents independently selected from the group consisting of alkanoyl, alkyl, alkoxy, alkoxycarbonyl, amido, amidoalkenyl, amidoalkyl, amino, aminoalkenyl, aminoalkyl, aryl, arylalkyl, carboxy, cyano, halo, hydroxy, hydroxyalkyl, nitro, oxo and thioalkoxy.

The term “(heterocycloalkyl)alkyl,” as used herein, represents a heterocycloalkyl group attached to the parent molecular moiety through an alkyl group.

The term “hydroxy,” as used herein, represents —OH.

The term “hydroxyalkyl,” as used herein, represents a hydroxy group attached the parent molecular moiety through an alkyl group.

The term “nitro,” as used herein, represents —NO₂.

The term “nitrogen protecting group,” as used herein, represents selectively introducible and removable groups which protect amino groups against undesirable side reactions during synthetic procedures. Examples of amino protecting groups include methoxycarbonyl, ethoxycarbonyl, trichloroethoxycarbonyl, benzyloxycarbonyl (Cbz), chloroacetyl, trifluoroacetyl, phenylacetyl, formyl, acetyl, benzoyl, tert-butoxycarbonyl (Boc), para-methoxybenzyloxycarbonyl, isopropoxycarbonyl, phthaloyl, succinyl, benzyl, diphenylmethyl, triphenylmethyl (trityl), methylsulfonyl, phenylsulfonyl, para-toluenesulfonyl, trimethylsilyl, triethylsilyl, triphenylsilyl, and the like.

The term “oxo,” as used herein, represents ═O.

The term “perfluoroalkoxy,” as used herein, represents a perfluoroalkyl group attached to the parent molecular moiety through an oxygen atom.

The term “perfluoralkyl,” as used herein, represents an alkyl group in which all of the hydrogen atoms have been replaced with fluoride atoms.

The term “phenyl,” as used herein, represents a 6 membered aromatic ring that is unsubstituted.

The term “phenylalkoxy,” as used herein, represents a phenyl group attached to the parent molecular moiety through an alkoxy group.

The term “phenylalkyl,” as used herein, represents a phenyl group attached to the parent molecular moiety through an alkyl group.

The term “phenylcarbonyl,” as used herein, represents a phenyl group attached to the parent molecular moiety through a carbonyl group.

The term“phenylsulfonyl,” as used herein, represents a phenyl group attached to the parent molecular moiety through a sulfonyl group.

The term “sulfonyl,” as used herein, represents —SO₂—.

The term “thioalkoxy,” as used herein, represents an alkyl group attached to the parent molecular moiety through a sulfur atom.

The instant compounds can exist as therapeutically acceptable salts. The term “therapeutically acceptable salt,” refers to salts or zwitterions of the compounds which are water or oil-soluble or dispersible, suitable for treatment of diseases without undue toxicity, irritation, and allergic response, commensurate with a reasonable benefit/risk ratio, and effective for their intended use. The salts can be prepared during the final isolation and purification of the compounds or separately by reacting an amino group of the compounds with a suitable acid. Representative salts include acetate, adipate, alginate, |citrate, aspartate, benzoate, benzenesulfonate, bisulfate, butyrate, camphorate, camphorsulfonate, digluconate, glycerophosphate, hemisulfate, heptanoate, hexanoate, formate, isethionate, fumarate, lactate, maleate, methanesulfonate, naphthylenesulfonate, nicotinate, oxalate, pamoate, pectinate, persulfate, 3-phenylpropionate, picrate, oxalate, maleate, pivalate, propionate, succinate, tartrate, trichloroacetic, trifluoroacetic, glutamate, para-toluenesulfonate, undecanoate, hydrochloric, hydrobromic, sulfuric, phosphoric, and the like. The amino groups of the compounds can also be quaternized with alkyl chlorides, bromides, and iodides such as methyl, ethyl, propyl, isopropyl, butyl, lauryl, myristyl, stearyl, and the like.

Basic addition salts can be prepared during the final isolation and purification of the instant compounds by reaction of a carboxyl group with a suitable base such as the hydroxide, carbonate, or bicarbonate of a metal cation such as lithium, sodium, potassium, calcium, magnesium, or aluminum, or an organic primary, secondary, or tertiary amine. Quaternary amine salts derived from methylamine, dimethylamine, trimethylamine, triethylamine, diethylamine, ethylamine, tributlyamine, pyridine, N,N-dimethylaniline, N-methylpiperidine, N-methylmorpholine, dicyclohexylamine, procaine, dibenzylamine, N,N-dibenzylphenethylamine, 1-ephenamine, and N,N′-dibenzylethylenediamine, ethylenediamine, ethanolamine, diethanolamine, piperidine, piperazine, and the like, are contemplated as being within the scope of the instant invention.

The instant compounds can also exist as therapeutically acceptable prodrugs. The term “therapeutically acceptable prodrug,” refers to those prodrugs or zwitterions which are suitable for use in contact with the tissues of patients without undue toxicity, irritation, and allergic response, are commensurate with a reasonable benefit/risk ratio, and are effective for their intended use. The term “prodrug,” refers to compounds which are rapidly transformed in vivo to the parent compounds of formula (I) for example, by hydrolysis in blood.

Asymmetric centers can exist in the instant compounds. Individual stereoisomers of the compounds are prepared by synthesis from chiral starting materials or by preparation of racemic mixtures and separation by conversion to a mixture of diastereomers followed by separation or recrystallization, chromatographic techniques, or direct separation of the enantiomers on chiral chromatographic columns. Starting materials of particular stereochemistry are either commercially available or are made by the methods described hereinbelow and resolved by techniques well-known in the art.

Geometric isomers can exist in the instant compounds The invention contemplates the various geometric isomers and mixtures thereof resulting from the disposal of substituents around a carbon-carbon double bond, a cycloalkyl group, or a heterocycloalkyl group. Substituents around a carbon-carbon double bond are designated as being of Z or E configuration and substituents around a cycloalkyl or heterocycloalkyl are designated as being of cis or trans configuration.

Therapeutic compositions of the instant compounds comprise an effective amount of the same formulated with one or more therapeutically acceptable excipients. The term “therapeutically acceptable excipient,” as used herein, represents a non-toxic, solid, semi-solid or liquid filler, diluent, encapsulating material, or formulation auxiliary of any type. Examples of therapeutically acceptable excipients include sugars; cellulose and derivatives thereof; oils; glycols; solutions; buffering, coloring, releasing, coating, sweetening, flavoring, and perfuming agents; and the like. These therapeutic compositions can be administered parenterally, intracisternally, orally, rectally, or intraperitoneally.

Liquid dosage forms for oral administration of the instant compounds comprise formulations of the same as emulsions, microemulsions, solutions, suspensions, syrups, and elixirs. In addition to the compounds, the liquid dosage forms can contain diluents and/or solubilizing or emulsifying agents. Besides inert diluents, the oral compositions can include wetting, emulsifying, sweetening, flavoring, and perfuming agents.

Injectable preparations of the instant compounds comprise sterile, injectable, aqueous and oleaginous solutions, suspensions or emulsions, any of which can be optionally formulated with parenterally acceptable diluents, dispersing, wetting, or suspending agents. These injectable preparations can be sterilized by filtration through a bacterial-retaining filter or formulated with sterilizing agents which dissolve or disperse in the injectable media.

PTP inhibition by the instant compounds can be delayed by using a liquid suspension of crystalline or amorphous material with poor water solubility. The rate of absorption of the compounds depends upon their rate of dissolution which, in turn, depends on their crystallinity. Delayed absorption of a parenterally administered compound can be accomplished by dissolving or suspending the compound in oil. Injectable depot forms of the compounds can also be prepared by microencapsulating the same in biodegradable polymers. Depending upon the ratio of compound to polymer and the nature of the polymer employed, the rate of release can be controlled. Depot injectable formulations are also prepared by entrapping the compounds in liposomes or microemulsions which are compatible with body tissues.

Solid dosage forms for oral administration of the instant compounds include capsules, tablets, pills, powders, and granules. In such forms, the compound is mixed with at least one inert, therapeutically acceptable excipient such as a carrier, filler, extender, disintegrating agent, solution retarding agent, wetting agent, absorbent, or lubricant. With capsules, tablets, and pills, the excipient can also contain buffering agents. Suppositories for rectal administration can be prepared by mixing the compounds with a suitable non-irritating excipient which is solid at ordinary temperature but fluid in the rectum.

The instant compounds can be micro-encapsulated with one or more of the excipients discussed previously. The solid dosage forms of tablets, dragees, capsules, pills, and granules can be prepared with coatings and shells such as enteric and release-controlling. In these forms, the compounds can be mixed with at least one inert diluent and can optionally comprise tableting lubricants and aids. Capsules can also optionally contain opacifying agents which delay release of the compounds in a desired part of the intestinal tract.

Transdermal patches have the added advantage of providing controlled delivery of the instant compounds to the body. Such dosage forms are prepared by dissolving or dispensing the compounds in the proper medium. Absorption enhancers can also be used to increase the flux of the compounds across the skin, and the rate of absorption can be controlled by providing a rate controlling membrane or by dispersing the compounds in a polymer matrix or gel.

Diseases caused or exacerbated by protein tyrosine phosphatase PTP1B activity are treated or prevented in a patient by administering to the same a therapeutically effective amount of the instant compounds in such an amount and for such time as is necessary to achieve the desired result. The term “therapeutically effective amount,” refers to a off sufficient amount of the compound to treat protein tyrosine phosphatase PTP I B activity at a reasonable benefit/risk ratio applicable to any medical treatment. The specific therapeutically effective dose level for any particular patient will depend upon a variety of factors including the disorder being treated and the severity of the disorder; the activity of the compound employed; the specific composition employed; the age, body weight, general health, sex, and diet of the patient; the time of administration, route of administration, rate of excretion; the duration of the treatment; and drugs used in combination or coincidental therapy.

The total daily dose of the instant compounds in single or divided doses can be in amounts, for example, from 0.01 to 50 mg/kg body weight or more usually from 0.1 to 25 mg/kg body weight. Single dose compositions can contain such amounts or submultiples thereof of the compounds to make up the daily dose. In general, treatment regimens comprise administration to a patient in need of such treatment from about 10 mg to about 1000 mg of the compounds per day in single or multiple doses.

Specific compounds of formula (II) include, but are not limited to:

2-((carboxycarbonyl)(1-naphthyl)amino)benzoic acid;

2-((carboxycarbonyl)(2-naphthyl)amino)benzoic acid;

2-((carboxycarbonyl)-4-methoxyanilino)benzoic acid;

2-((carboxycarbonyl)(1-naphthyl)amino)-5-methoxybenzoic acid;

2-((carboxycarbonyl)-2-chloro-5-methoxyanilino)benzoic acid;

2-((carboxycarbonyl)-2-((1E)-3-ethoxy-3-oxo-1-propenyl)anilino)benzoic acid;

2-(4-((2S)-2-((tert-butoxycarbonyl)amino)-3-(((4-(methoxycarbonyl)cyclohexyl)methyl)amino)-3-oxopropyl) (carboxycarbonyl)anilino)benzoic acid;

2-(4-((2 S)-2-((tert-butoxycarbonyl)amino)-3 -(((4-carboxycyclohexyl)methyl)amino)-3-oxopropyl) (carboxycarbonyl)anilino)benzoic acid;

2-((carboxycarbonyl)-2-iodoanilino)benzoic acid;

2-((carboxycarbonyl)-2-(( 1E)-3-( 1,3-oxazinan-3-yl)-3-oxo-1-propenyl)anilino)benzoic acid;

2-((carboxycarbonyl)-3-(trifluoromethyl)anilino)benzoic acid;

2-((carboxycarbonyl)(cyclobutyl)amino)benzoic acid;

2-((7-(benzyloxy)-1-naphthyl)(carboxycarbonyl)amino)benzoic acid,

2-((carboxycarbonyl)-2-(2-hydroxyethyl)anilino)benzoic acid;

2-((carboxycarbonyl)-2-methylanilino)benzoic acid;

2-((carboxycarbonyl)(2-methyl-1H-indol-1-yl)amino)benzoic acid;

2-((carboxycarbonyl)(7-hydroxy-1-naphthyl)amino)benzoic acid;

2-((carboxycarbonyl)(7-((6-phenylhexyl)oxy)-1-naphthyl)amino)benzoic acid;

2-((1,1′-biphenyl)-2-yl(carboxycarbonyl)amino)benzoic acid;

2-((1,1′-biphenyl)-4-yl(carboxycarbonyl)amino)benzoic acid;

2-((carboxycarbonyl)(5,6,7,8-tetrahydro-1-naphthalenyl)amino)benzoic acid;

2-((carboxycarbonyl)(cyclohexyl)amino)benzoic acid;

2-((carboxycarbonyl)(2,3-dihydro-1,4-benzodioxin-6-yl)amino)benzoic acid;

2-((carboxycarbonyl)(3-methylcyclohexyl)amino)benzoic acid;

2-[(carboxycarbonyl)(2-hydroxy-1-naphthyl)amino]benzoic acid;

N-acetyl-4-[(carboxycarbonyl)(2-carboxyphenyl)amino]-N-pentyl-1-naphthylalaninamide;

N-acetyl-4-[(carboxycarbonyl)(2-carboxyphenyl)amino]-N-pentyl-3-(2-hydroxyethane)-phenylalaninamide;

4-[(carboxycarbonyl)(2-carboxyphenyl)amino]-6-{[N-acetyl-3-(1-naphthyl)alanyl]amino}hexanoic acid;

4-[(carboxycarbonyl)(2-carboxyphenyl)amino]-3-[(1 E)-3-amino-3-oxo-1-propenyl]-N-(tert-butoxycarbonyl)-N-pentyl-L-phenylalaninamide;

N-acetyl-4-[(carboxycarbonyl)(2-carboxyphenyl)amino]-3-isopropyl-N-pentylphenylalaninamide;

4-[(carboxycarbonyl)(2-carboxyphenyl)amino]-6-{[N-acetyl-3-(1-piperidinyl)phenylalanyl]amino}hexanoic acid;

2-{(carboxycarbonyl)[2-(3-methyl-1-piperidinyl)phenyl]amino}benzoic acid;

2-{(carboxycarbonyl)[5-hydroxy-2-(1-piperidinyl)phenyl]amino}benzoic acid;

4-[(carboxycarbonyl)(2-carboxyphenyl)amino]-3-[(1E)-3-amino-3-oxo-1-propenyl]-N-(methylsulfonyl)-N-pentyl-L-phenylalaninamide;

4-[(carboxycarbonyl)(2-carboxyphenyl)amino]-3-(3-amino-3-oxopropyl)-N-[(isopropylamino)carbonyl]-N-pentyl-L-phenylalaninamide;

4-[(carboxycarbonyl)(2-carboxyphenyl)amino]-3-[(1 E)-3-amino-3-oxo-1-propenyl]-N-[(isopropylamino)carbonyl]-N-pentyl-L-phenylalaninamide;

2-((carboxycarbonyl) {2-[4-(hydroxymethyl)-1-piperidinyl]phenyl}amino)benzoic acid;

N-acetyl-4-[(carboxycarbonyl)(2-carboxyphenyl)amino]-N-pentyl-4-( 1-piperidinyl)phenylalaninamide;

N-acetyl-4-[(carboxycarbonyl)(2-carboxyphenyl)amino]-3,-ethylphenylalanyl-N-methyl-4-nitro-L-phenylalaninamide;

N-(3-carboxypropanoyl)-L-phenylalanyl-3-[(1E)-3-amino-3-oxo-1-propenyl]-4-[(carboxycarbonyl)(2-carboxyphenyl)amino]-N-pentyl-L-phenylalaninamide;

3-(4-benzoylphenyl)-N-(tert-butoxycarbonyl)-L-alanyl-3-{4-[(carboxycarbonyl)(2-carboxyphenyl)amino]phenyl}-N-1--pentyl-L-alaninamide;

N-acetyl-4-[(carboxycarbonyl)(2-carboxyphenyl)amino]-3-(2-hydroxyethyl)-N-[4-(methylsulfonyl)benzyl]phenylalaninamide;

2-[[7-(aminocarbonyl)-1-naphthyl](carboxycarbonyl)amino]benzoic acid;

N-acetyl-4-[(carboxycarbonyl)(2-carboxyphenyl)amino]-3-isopropyl-N-[4-(methylsulfonyl)benzyl]phenylalaninamide

4-[(carboxycarbonyl)(2-carboxyphenyl)amino]-1-acetyl-6-(3-isopropylbenzyl)-4-[4-(methylsulfonyl)benzyl]-2,3,5-piperazinetrione;

2-[(carboxycarbonyl)(7-hydroxy-1-naphthyl)amino]-4-hydroxybenzoic acid;

N-acetyl-4-[(carboxycarbonyl)(2-carboxyphenyl)amino]-3-ethyl-N-{5-oxo-5-[(1-phenylethyl)amino]pentyl}phenylalaninamide;

N-(methoxycarbonyl)-4-[(carboxycarbonyl)(2-carboxyphenyl)amino]-N-pentylnaphthylalaninamide;

4-[(carboxycarbonyl)(2-carboxyphenyl)amino]-N-(cyclohexylmethyl)-N-(methoxycarbonyl)naphthylalaninamide;

4-[(carboxycarbonyl)(2-carboxyphenyl) amino]-N-(methoxycarbonyl)-N-[(1R)-1-(4-nitrophenyl)ethyl]naphthylalaninamide;

4-[(carboxycarbonyl)(2-carboxyphenyl)amino]-N-(methoxycarbonyl)-N-[4-(methylsulfonyl)benzyl]naphthylalaninamide;

4-[(carboxycarbonyl)(2-carboxyphenyl)amino]-N-(methoxycarbonyl)-N-(3,4,5-trifluorobenzyl)naphthylalaninamide;

4-[(carboxycarbonyl)(2-carboxyphenyl)amino]-N-(cyclooctylmethyl)-N-(methoxycarbonyl)naphthylalaninamide;

4-[(carboxycarbonyl)(2-carboxyphenyl)amino]-N-[(1 R)-1-(4-bromophenyl)ethyl]-N-(methoxycarbonyl)naphthylalaninamide;

4-[(carboxycarbonyl)(2-carboxyphenyl)amino]-N-(methoxycarbonyl)-N-(3-phenylpropyl)naphthylalaninamide;

methyl 3-{4-[(carboxycarbonyl)(2-carboxyphenyl)amino]-1-naphthyl}-N-(methoxycarbonyl)alanyl-L-norleucinate;

4-[(carboxycarbonyl)(2-carboxyphenyl)amino]-N-(2-fluorobenzyl)-N-(methoxycarbonyl)naphthylalaninamide;

4-[(carboxycarbonyl)(2-carboxyphenyl)amino]-N-(4-chlorobenzyl)-N-(methoxycarbonyl)naphthylalaninamide;

4-[(carboxycarbonyl)(2-carboxyphenyl)amino]-N-(4-bromobenzyl)-N-(methoxycarbonyl)naphthylalaninamide;

4-[(carboxycarbonyl)(2-carboxyphenyl)amino]-N-(methoxycarbonyl)-N-(4-nitrobenzyl)naphthylalaninamide;

4-[(carboxycarbonyl)(2-carboxyphenyl)amino]-N-[4-(aminosulfonyl)benzyl]-N-(methoxycarbonyl)naphthylalaninamide;

4-[(carboxycarbonyl)(2-carboxyphenyl)amino]-N-(methoxycarbonyl)-N-({4-[(methylamino)carbonyl]cyclohexyl}methyl)naphthylalaninamide;

N-acetyl-4-[(carboxycarbonyl)(2-carboxyphenyl)amino]-3-(2-hydroxyethyl)-N-(4-nitrobenzyl)phenylalaninamide;

2-[(carboxycarbonyl)(7-hydroxy-1-naphthyl)amino]-4-cyanobenzoic acid;

4-[(carboxycarbonyl)(2-carboxyphenyl)amino]-N-{4-[(ethylamino)sulfonyl]benzyl}-N-(methoxycarbonyl)naphthylalaninamide;

N-(tert-butoxycarbonyl)-L-phenylalanyl-3-[(1E)-3-amino-3-oxo-1-propenyl]-4-[(carboxycarbonyl)(2-carboxyphenyl)amino]-N-pentyl-L-phenylalaninamide;

N-acetyl-4-[(carboxycarbonyl)(2-carboxyphenyl)amino]-3-(2-hydroxyethyl)-N-[(1S)-1-(4-nitrophenyl)ethyl]phenylalaninamide;

N-acetyl-4-[(carboxycarbonyl)(2-carboxyphenyl)amino]-N-(4-chlorobenzyl)-3-(2-hydroxyethyl)phenylalaninamide;

N-acetyl-4-[(carboxycarbonyl)(2-carboxyphenyl)amino]-N-(4-bromobenzyl)-3-(2-hydroxyethyl)phenylalaninamide;

2-[(carboxycarbonyl)(7-hydroxy-1-naphthyl)amino]-4-{[4-(dimethylamino)benzoyl]amino}benzoic acid;

N-acetyl-4-[(carboxycarbonyl)(2-carboxyphenyl)amino]-3-ethyl-N-(4-nitrobenzyl)phenylalaninamide;

N-acetyl-4-[(carboxycarbonyl)(2-carboxyphenyl)amino]-3-ethyl-N-[(1 R)-1-(4-nitrophenyl)ethyl]phenylalaninamide;

N-acetyl-4-[(carboxycarbonyl)(2-carboxyphenyl)amino]-N-(4-chlorobenzyl)-3-ethylphenylalaninamide;

N-acetyl-4-[(carboxycarbonyl)(2-carboxyphenyl)amino]-N-(4-nitrobenzyl)naphthylalaninamide;

N-acetyl-4-[(carboxycarbonyl)(2-carboxyphenyl)amino]-N-[(1R)-1-(4-bromophenyl)ethyl]-3-(2-hydroxyethyl)phenylalaninamide;

4-[(butylamino)carbonyl]-2-[(carboxycarbonyl)(7-hydroxy-1-naphthyl)amino]benzoic acid;

N-acetyl-4-[(carboxycarbonyl)(2-carboxyphenyl)amino]-N-{5-[(3-hydroxyphenyl)amino]-5-oxopentyl}-3-(1-piperidinyl)phenylalaninamide;

2-((carboxycarbonyl){4-[2-hydroxy-3-(pentylamino)propyl]phenyl}amino)benzoic acid;

2-((carboxycarbonyl){4-[3-(pentylamino)butyl]1-naphthyl}amino)benzoic acid; and

2-((carboxycarbonyl){4-[3-(pentylamino)propyl]-1-naphthyl}amino)benzoic acid.

The following additional compounds, representative of formula (II), may be prepared by one skilled in the art using known synthetic methodology or by using synthetic methodology described in the Schemes and Examples contained herein.

2-((carboxycarbonyl)-2-((E)-2-carboxyethenyl)anilino)benzoic acid;

2-(2-((1E)-3-((tert-butoxycarbonyl)amino)-1-propenyl)(carboxycarbonyl)anilino)benzoic acid;

2-((carboxycarbonyl)-2,3-dimethylanilino)benzoic acid;

2-((carboxycarbonyl)-4-chloro-3-methylanilino)benzoic acid;

2-(2-(aminocarbonyl)(carboxycarbonyl)anilino)benzoic acid;

2-((7-(aminomethyl)-5,6,7,8-tetrahydro-1-naphthalenyl)(carboxycarbonyl)amino)benzoic acid;

2-((6-(aminomethyl)-5,6,7,8-tetrahydro-1-naphthalenyl)(carboxycarbonyl)amino)benzoic acid;

2-((carboxycarbonyl)-4-(2,3-diamino-3-oxopropyl)anilino)benzoic acid; and

2-((carboxycarbonyl)(5-(2,3-diamino-3-oxopropyl)-5,6,7,8-tetrahydro-1-naphthalenyl)amino)benzoic acid.

Determination of Biological Activity

Purification of Human Protein Tyrosine Phosphatase 1B from E. coli

Human protein tyrosine phosphatase 1B (PTP1B, amino acid residues 1-321) was expressed in E. coli BL21(DE3). The cell paste was resuspended in 4 cell paste volumes of lysis buffer containing 100 mM MES (pH 6.5), 100 mM NaCl, 1 mM EDTA, 1 mM DTT, 1 mM PMSF, 20 U/mL Benzonase, 0.5 mg/mL lysozyme, and 1 mM MgCl₂ and incubated for 35 minutes at room temperature. The cells were lysed at 11,000 psi using a Rannie homogenizer, and the homogenate was clarified in a Beckman GSA rotor at 10,000×g for 30 minutes at 4° C. The supernatant was loaded onto a 5×21 cm S-Sepharose-FF column (Amersham Pharmacia Biotech) pre-equilibrated with 5 column volumes of buffer containing 100 mM MES (pH 6.5), 100 mM NaCl, 1 mM EDTA, and I mM DTT and lo eluted with 10 column volumes of the same. The fractions (28 mL each) were assayed for protein by 10-20% Tris-Glycine SDS-PAGE. Fractions which contained >95% protein tyrosine phosphatase 1B were combined.

Protein Tyrosine Phosphatase 1B Activity Assay

Protein tyrosine phosphatase 1B activity was determined by measuring the phosphate release from triphosphorylated peptide which corresponds to residues 1135-1156 of the β-subunit of the human insulin receptor (βIRK substrate) as described in Nature, 1985, 313, 756-761. Protein tyrosine phosphatase 1B activity was determined in a final assay volume of 50 μL containing 50 mM Tris HCl, 50 mM Tris Base, 150 mM NaCl, 3mM DTT, 2 nM protein tyrosine phosphatase 1B(1-321), and 20 μM βIRK substrate. Various concentrations of test compounds in 5 μL of 10% DMSO were incubated for 5 minutes at room temperature in assay buffer (25 μl) containing 20 μM βIRK substrate in a round-bottom microtiter plate(Costar) pre-coated with 1% bovine serum albumin. The assay was initiated by the addition of protein tyrosine phosphatase 1B enzyme (20 μl )in assay buffer. After 10 minutes of incubation at room temperature, the reaction was terminated by the addition of 100 μL of malachite green (Upstate Biotechnology Inc.) containing 0.01% Tween-20. After a 5 minute incubation, quantitation of free phosphate released from the βIRK substrate was determined in a Victor II plate reader (Wallac; Turku, Finland) by measuring the absorbence of the malachite green at 620 nm.

The instant compounds were found to inhibit protein tyrosine phosphatase 1B with inhibitory potencies in a range of about 0.05 μM to about 100 μM. In a preferred range, the compounds inhibited protein tyrosine phosphatase 1B with inhibitory potencies in a range of about of about 0.05 μM to about 60 μM; and in a more preferred range, the compounds inhibited protein tyrosine phosphatase 1B with inhibitory potencies in a range of about of about 0.05 μM to about 21 μM.

As protein tyrosine phosphatase 1B inhibitors, therefore, the instant compounds are useful for treating diseases caused by overexpressed or altered protein tyrosine phosphatase 1B activity. These diseases include autoimmune diseases, acute and chronic inflammatory diseases, osteoporosis, obesity, cancer, malignant diseases, and type I and type II diabetes.

Synthetic Methods

Abbreviations which have been used in the descriptions of the scheme and the examples that follow are: dba for dibenzylideneacetone; DMSO for dimethylsulfoxide; NMP for N-methylpyrrolidinone; DMF for N,N-dimethylformamide; TFA for trifluoroacetic acid; THF for tetrahydrofuran; EDAC for 1-(3-dimethylaminopropyl)-3-ethylcarbodiimide hydrochloride; and HOBT for 1-hydroxybenzotriazole hydrate.

The compounds and processes of the instant invention will be better understood in connection with the following synthetic schemes which illustrate the methods by which the compounds of the invention may be prepared. The groups R¹, R², R³, R⁴, R⁵, and R⁶ are as defined above unless otherwise noted below.

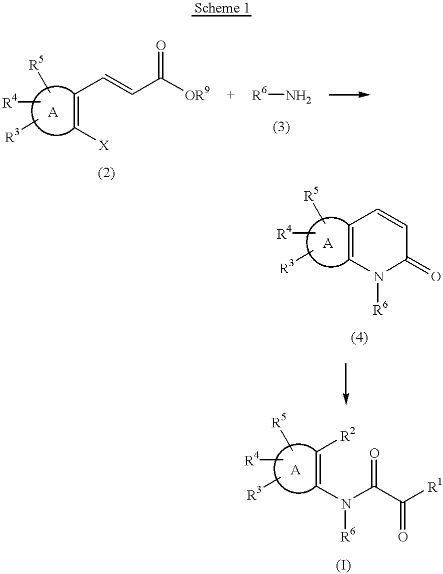

As shown in Scheme 1, compounds of formula (2) (R⁹ is alkyl; X is Br or I) can be reacted with compounds of formula (3) (R⁶ is aryl or heteroaryl) in the presence of a palladium catalyst and base to form compounds of formula (4). Representative palladium catalysts include Pd₂dba₃ with 2-dicyclohexylphosphino-2′-(N,N-dimethyl)aminobiphenyl, Pd₂dba₃ with tricyclohexylphosphine, and Pd₂dba₃ with PPh₃. Representative bases include sodium hydride, potassium hydride, and calcium hydride. Examples of solvents used in these reactions include benzene and toluene. The reaction temperature is about 60° C. to about 110° C. and depends on the method chosen. Reaction times are typically about 2 to about 8 hours.

Compounds of formula (4) can be converted to compounds of formula (I) (R¹ is hydroxy, R² is carboxy) by treatment with an oxidizing agent. Representative oxidizing agents include KMnO₄, ozone and hydrogen peroxide, and CrO₃. Examples of solvents used in these reactions include pyridine, water, and mixtures thereof. The reaction temperature is about 0° C. to about 35° C. and depends on the method chosen. Reaction times are typically about 12 to about 24 hours.

Compounds of formula (I) (R¹ is hydroxy and R² is carboxy) can be intraconverted to compounds of formula (I) (R¹ is alkoxy and R² are alkoxycarbonyl) by methods well known to those skilled in the art.

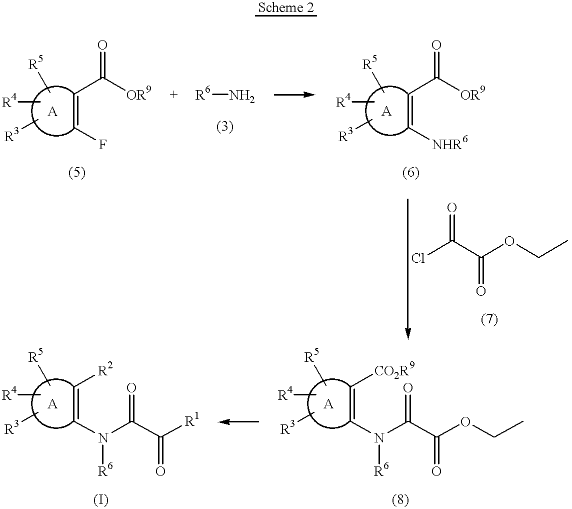

An alternative synthesis of compounds of formula (I) is shown in Scheme 2. Compounds of formula (5) (R⁹ is alkyl) can be reacted with compounds of formula (3) (R⁶ is alkyl, arylalkyl, cycloalkenyl, cycloalkenylalkyl, cycloalkyl, cycloalkylalkyl, heteroarylalkyl, heterocycloalkyl, or (heterocycloalkyl)alkyl to provide compounds of formula (6). Examples of solvents used in these reactions include DMSO, dioxane, and NMP. The reaction temperature is about 80° C. to about 120° C. Reaction times are typically about 12 to about 24 hours.

Compounds of formula (6) can be reacted with ethyl chloro(oxo)acetate (7) in the presence of base to provide compounds of formula (8). Representative bases include pyridine, triethylamine, and diisopropylethylamine. Examples of solvents used in these reactions include diethyl ether, methyl tert-butyl ether, and dioxane. The reaction temperature is about 20° C. to about 30° C. Reaction times are typically about 8 to about 18 hours.

Compounds of formula (8) can be hydrolyzed to compounds of formula (I) (R¹ is hydroxy and R² is carboxy) by methods known to those skilled in the art.

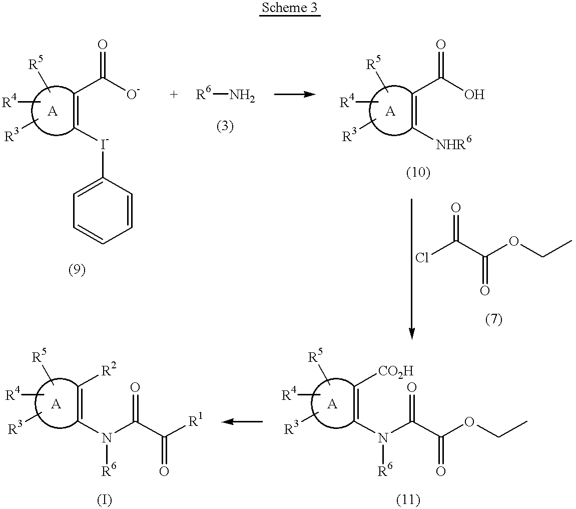

Another synthesis of compounds of formula (I) is shown in Scheme 3. Compounds of formula (9) can be reacted with compounds of formula (3) (R⁶ is aryl or heteroaryl) in the presence of catalytic copper(II) acetate to provide compounds of formula (10). Examples of solvents used in these reactions include isopropanol, n-propanol, butanol, and pentanol. The reaction temperature is about 70° C. to about 100° C. Reaction times are typically about 4 to about 12 hours.

Compounds of formula (10) can be converted to compounds of formula (11) and then to compounds of formula (I) (R¹ is hydroxy and R² is carboxy) by the methods described in Scheme 2.

The instant invention will now be described in connection with certain preferred embodiments which are not intended to limit its scope. On the contrary, the instant invention covers all alternatives, modifications, and equivalents as can be included within the scope of the claims. Thus, the following examples, which include preferred embodiments, will illustrate the preferred practice of the instant invention, it being understood that the examples are for the purposes of illustration of certain preferred embodiments and are presented to provide what is believed to be the most useful and readily understood description of its procedures and conceptual aspects.

Compounds of the invention were named by ACD/ChemSketch version 4.01 (developed by Advanced Chemistry Development, Inc., Toronto, ON, Canada) or were given names which appeared to be consistent with ACD nomenclature.

### EXAMPLE 1

### 2-((carboxycarbonyl)(1-naphthyl)amino)benzoic Acid

### Example 1A

### Methyl (2E)-3-(2-bromophenyl)-2-propenoate

A solution of (2E)-3-(2-bromophenyl)-2-propenoic acid (1.53g, 6.74 mmol) in N,N-dimethylformamide (10 mL) at room temperature was treated with K₂CO₃ (900 mg, 6.51 mmol) and iodomethane (0.5 mL, 8.0 mmol), stirred for 3 hours, poured into H₂O (50 mL) and extracted with diethyl ether. The combined extracts were washed with water and brine, dried (MgSO₄), filtered, and concentrated to provide the desired product.

### Example 1B

### Ethyl (2E)-3-(2-bromophenyl)-2-propenoate

A mixture of Example 1A (135 mg, 0.56 mmol), 1-aminonaphthalene (80 mg, 0.56 mmol), tris(dibenzylideneacetone)-dipalladium(0) (3 mg, 0.003 mmol), 2-dicyclohexylphosphino-2′-(N,N-dimethyl)aminobiphenyl (4 mg, 0.01 mmol), and 60% NaH dispersion in mineral oil (50 mg, 1.2 mmol) in toluene (2 mL) was heated to reflux for 5.5 hours diluted with water (10 mL) and 1N HCl (5 mL), and extracted with ethyl acetate. The combined extracts were washed with water and brine, dried (MgSO₄), filtered, and concentrated. The concentrate was purified by flash column chromatography on silica gel with 1:1 ethyl acetate/hexanes to provide the desired product. MS (ESI(+)) m/e 272 (M+H)⁺; ¹H NMR (300 MHz, DMSO-d₆) δ8.16 (d, 2H), 8.12 (d, 1H), 7.84 (dd, 1H), 7.74 (dd, 1H), 7.60 (dd, 1H), 7.57 (dt, 1H), 7.46 (ddd, 1H), 7.32 (ddd, 1H), 7.25 (dd, 1H), 7.20 (dd, 1H), 6.78 (d, 1H), 6.30 (d, 1H).

### Example 1C

### 2-((carboxycarbonyl)(1-naphthyl)amino)benzoic Acid

A solution of Example 1B (97 mg, 0.36 mmol) in pyridine (1.2 mL) at room temperature was treated with water (1.2 mL), cooled to 0° C., treated with KMnO₄ (210 mg, 1.3 mmol), warmed to room temperature, and stirred for 17 hours. The mixture was treated with methanol (0.2 mL), stirred for 5 minutes, treated with 1N NaOH (4 mL), and filtered through diatomaceous earth (Celite®). The filter cake was washed with water (15 mL) and the combined filtrates were washed with diethyl ether, cooled to 0° C., adjusted to pH <7 with 12N HCl, and extracted with ethyl acetate. The combined extracts were washed with brine, dried (MgSO₄), filtered, and concentrated. The concentrate was 5 purified by reverse-phase HPLC with 100% acetonitrile to 70:30 (0.1% aqueous trifluoroacetic acid)/acetonitrile provide the desired product as a 3:2 mixture of rotamers. MS (ESI(+)) m/e 336 (M+H)⁺; ¹H NMR (500 MHz, DMSO-d₆) 6 8.37 (d, 1H), 8.05-8.03(m, 1H), 8.01-7.95 (m, 1H), 7.87 (dd, 1H), 7.73-7.68 (m, 1H), 7.63-7.59 (m, 1H), 7.57-7.49 (m, 2H), 7.45-7.38 (m, 1H), 7.34 (dt, 1H), 7.21 (d, 1H), 6.89 (d, 1H).

### EXAMPLE 2

### 2-((carboxycarbonyl)(2-naphthyl)amino)benzoic Acid

The titled compound was prepared by substituting 2-aminonaphthalene for 1-aminonaphthalene in Example 1. MS(ES(+)) m/e 336 (M+H)⁺; ¹H NMR (300 MHz, DMSO-d₆) δ7.98-7.82 (m, 4H), 7.72-7.62 (m, 3H), 7.58-7.45 (m, 4H).

### EXAMPLE 3

### 2-((carboxycarbonyl)-4-methoxyanilino)benzoic Acid

A mixture of 4-methoxyaniline (246 mg, 2 mmol), diphenyliodonium-2-carboxylate monohydrate (821 mg, 2.4 mmol), copper(II) acetate (18.2 mg, 0.1 mmol), and isopropanol (4 ml) in a sealed tube under nitrogen atmosphere was heated to 80° C. for 8 hours, cooled to room temperature, treated with 1N NaOH (3 mL), and extracted with diethyl ether. The aqueous phase was adjusted to pH 2 with 1N HCl and extracted with ethyl acetate. The combined extracts were filtered through a pad of silica gel, dried (Na₂SO₄), filtered, and concentrated. A solution of the concentrate (427 mg, 1.75 mmol) and diisopropylethylamine (762 μL, 4.38 mmol) in dichloromethane (5 mL) was cooled to 0° C., treated slowly with ethyl oxalyl chloride (450 μL, 4.03 mmol), stirred for 1 hour, warmed to room temperature, stirred for 16 hours, treated with 1N HCl (4 mL), and extracted with ethyl acetate. The combined extracts were dried (Na₂SO₄), filtered, and concentrated. The concentrate was purified by flash column chromatography on silica gel with 50:50:0.8 ethyl acetate/hexanes/acetic acid. A solution of the purified concentrate (86 mg, 0.25 mmol) in ethanol (2 mL) at room temperature was treated with 1N NaOH (1 mL), stirred for 30 minutes, treated with 1N HCl (2 mL), and purified by reverse-phase HPLC with acetonitrile to 95:5 (0.1% aqueous trifluoroacetic acid)/ acetonitrile provide 35 the desired product. MS (ESI(−)) m/e 314 (M−H)⁻; ¹H NMR (300 MHz, DMSO-d₆ at 90° C.) δ7.84 (dd, 1H), 7.57 (td, 1H), 7.25-7.48 (br m, 4H), 6.91 (d, 2H), 3.74 (s, 3H).

### EXAMPLE 4

### 2-((carboxycarbonyl)(1-naphthyl)amino)-5-methoxybenzoic Acid

### Example 4A

### ethyl (2E)-3-(2-bromo-5 -methoxyphenyl)-2-propenoate

A suspension of 60% NaH dispersion in mineral oil (220 mg, 5.5 mmol) in THF (10 mL) at room temperature was treated dropwise with triethyl phosphonoacetate (1.14 mL, 5.75 mmol), stirred for 5 minutes, treated dropwise with a mixture of 2-bromo-5-methoxybenzaldehyde (1.07 g, 5.0 mmol) in THF (2 mL), stirred for 16 hours, and concentrated under reduced pressure. The concentrate was dissolved in ethyl acetate, washed with water and brine, dried (MgSO₄), filtered, and concentrated. The concentrate was purified by flash column chromatography on silica gel with 90:10 hexanes/ethyl acetate to provide the titled compound.

### Example 4B

### 2-((carboxycarbonyl)(1-naphthyl)amino)-5-methoxybenzoic Acid

The desired product was prepared according to the procedure described in Example 1B-C as a mixture of rotamers by substituting ethyl (2E)-3-(2-bromo-5-methoxyphenyl)-2-propenoate for methyl (2E)-3-(2-bromophenyl)-2-propenoate. MS (ESI(−)) m/e 364 (M−H)⁻; ¹H NMR (400 MHz, DMSO-d₆) δ8.38-8.35 (m, 1H), 8.05-7.83(m, 3H), 7.40 (d, 1H), 7.36 (d, 1H), 7.18 (d, 1H), 7.07 (dd, 1H), 6.96 (dd, 1H), 6.84 (d, 1H), 3.76 ( s,3H), 3.78 (s,3H).

### EXAMPLE 5

### 2-((carboxycarbonyl)-2-chloro-5-methoxyanilino)benzoic Acid

### Example 5A

### 2-(2-chloro-5-methoxyanilino)benzoic Acid

A mixture of 2-chloro-5-methoxyaniline (260 mg, 1.65 mmol), diphenyliodonium-2-carboxylate monohydrate (684 mg, 2.0 mmol), and copper(II) acetate (15 mg, 0.83 mmol) in 2-propanol (4 mL) was heated to reflux for 1.5 hours, diluted with 1N NaOH (10 mL), and extracted with hexanes. The aqueous phase was adjusted to pH <7 with 1M HCl and extracted with ethyl acetate. The combined extracts were washed with brine, dried (MgSO₄), filtered, and concentrated to provide the desired product.

### Example 5B

### 2-((carboxycarbonyl)-2-chloro-5-methoxyanilino)benzoic Acid

A solution of Example 5A (354 mg, 1.27 mmol) and pyridine (0.5 mL) in ethyl acetate (5 mL) at 0° C. was treated with ethyl chloro(oxo)acetate (350 mL, 3.1 mmol), warmed to room temperature, stirred for 2.5 hours, and poured into 1N HCl (20 mL). The aqueous phase was extracted with diethyl ether and the combined extracts were washed with water and 2N NaOH. The combined NaOH washes were adjusted to pH <7 with 1N HCl (20 mL), and extracted with ethyl acetate. This combined extracts were washed with brine, dried (MgSO₄), filtered, and concentrated. The concentrate was purified by reverse phase HPLC with acetonitrile to 70:30 (0.1% aqueous trifluoroacetic acid)/ acetonitrile to provide the desired product as a mixture of rotamers. MS (ESI(+)) m/e 350 (M+H)⁺; ¹H NMR (300 MHz, DMSO-d₆) δ7.90-7.84 (m, 1H), 7.62-7.40 (m, 3H), 7.35 (d, 1H), 7.25-7.08 (m, 1H), 7.05-6.97 (m, 2H), 6.91 (s, 1H), 6.88-6.84 (m, 1H).

### EXAMPLE 6

### 2-((carboxycarbonyl)-2-(( 1E)-3-ethoxy-3-oxo-1-propenyl)anilino)benzoic Acid

The desired product was prepared by substituting ethyl (2E)-3-(2-aminophenyl)-2-propenoate for 4-methoxyaniline in Example 3. MS (ESI(−)) m/e 382 (M−H)⁻; ¹H NMR (300 MHz, DMSO-d₆) δ7.94 (d, 1H), 7.91-7.77 (m, 2H), 7.50-7.16 (m, 5H), 6.87 (d, 1H), 6.70 (d, 1H), 4.18 (q, 2H), 1.24 (t, 3H).

### EXAMPLE 7

### 2-(4-((2S)-2-((tert-butoxycarbonyl)amino)-3-(((4-(methoxycarbonyl)cyclohexyl)methyl)amino)-3-oxopropyl)(carboxycarbonyl)anilino)benzoic Acid

### Example 7A

### Methyl 4-((((2S)-3-(4-aminophenyl)-2-((tert-butoxycarbonyl)amino)propanoyl)amino)methyl)cyclohexane-carboxylate

A solution of (2S)-3-(4-aminophenyl)-2-((tert-butoxycarbonyl)amino)propanoic acid (350 mg, 1.25 mmol), methyl 4-(aminomethyl)cyclohexanecarboxylate hydrochloride (285 mg, 1.50 mmol), 1-[3-(dimethylamino)propyl]-3-ethylcarbodiimide hydrochloride (335 mg, 1.75 mmol), and 1-hydroxybenzotriazole hydrate (245 mg, 1.50 mmol) in N,N-dimethylformamide (6 mL) at room temperature was adjusted to pH 6 with triethylamine (250 μL, 1.78 mmol), stirred for 16 hours, treated with water, and filtered. The solid was washed with water, concentrated under reduced pressure and dried under vacuum to provide the desired product.

### Example 7B

### 2-(4-((2S)-2-((tert-butoxycarbonyl)amino)-3-(((4-(methoxycarbonyl)cyclohexyl)methyl)amino)-3-oxopropyl)(carboxycarbonyl)anilino)benzoic Acid

The desired product was prepared by substituting Example 7A for 4-methoxyaniline and substituting tert butyl oxalyl chloride for ethyl oxalyl chloride in Example 3. Purification by reverse-phase HPLC with 5%-100% acetonitrile/H₂O with 0.1% trifluoracetic acid provided the desired product as a mixture of rotamers. MS (ESI(−)) m/e 624 (M−H)⁻; ¹H NMR (300 MHz, DMSO-d₆) 6 7.93-7.73(m, 2H), 7.68-7.32 (m, 4H), 7.32-7.05 (m, 3H), 7.05-6.83 (m, 1H), 4.18-4.01 (m, 1H), 3.80 (s, 3H), 2.97-2.79 (m, 3H), 2.79-2.66 (m, 1H), 2.08 (t, 1H), 1.85 (br d, 1H), 1.78 (br d, 2H), 1.42-1.20 (m, 9H), 0.88 (t, 2H), 0.85 (t, 2H).

### EXAMPLE 8

### 2-(4-((2S)-2-((tert-butoxycarbonyl)amino)-3-(((4-carboxycyclohexyl)methyl)amino)-3-oxopropyl)(carboxycarbonyl)anilino)benzoic Acid

The desired product was isolated in the HPLC purification of Example 7B as a mixture of rotamers. MS (ESI(+)) m/e 612 (M+H)⁺; ¹H NMR (300 MHz, DMSO-d₆) δ7.93-7.73(m, 2H), 7.68-7.32 (m, 4H), 7.32-7.05 (m, 3H), 7.05-6.83(m, 1H), 4.18-4.01 (m, 1H), 2.97-2.79 (m, 3H), 2.79-2.66 (m, 1H), 2.08 (t, 1H), 1.85 (br d, 1H), 1.78 (br d, 2H), 1.42-1.20 (m, 9H), 0.88 (t, 2H), 0.85 (t, 2H).

### EXAMPLE 9

### 2-((carboxycarbonyl)-2-iodoanilino)benzoic Acid

The desired product was prepared by substituting 2-iodoaniline for 4-methoxyaniline in Example 3. MS (ESI(+)) m/e 412 (M+H)⁺; ¹H NMR (300 MHz, DMSO-d₆) 6 7.95 (dd, 1H), 7.82 (dd, 1H), 7.50-7.40 (m, 3H), 7.37 (dd, 1H), 7.31 (d, 1H), 7.04 (td, 1H).

### EXAMPLE 10

### 2-((carboxycarbonyl)-2-((1E)-3-(1,3-oxazinan-3-yl)-3-oxo-1-propenyl)anilino)benzoic Acid

The desired product was prepared by substituting 2-((1E)-3-(4-morpholinyl)-3-oxo-1-propenyl)phenylamine for 4-methoxyaniline in Example 3. MS (ESI(+)) m/e 425 (M+H)⁺; ¹H NMR (300 MHz, DMSO-d₆) δ7.93-7.87 (m, 1H), 7.76 (d, 1H), 7.48-7.20 (m, 7H), 7.16 (d, 1H), 3.62 (br m, 8H).

### EXAMPLE 11

### 2-((carboxycarbonyl)-3-(trifluoromethyl)anilino)benzoic Acid

The desired product was prepared by substituting 3-trifluoromethylaniline for 4-methoxyaniline in Example 3. MS (ESI(−)) m/e 352 (M−H)⁻; ¹H NMR (300 MHz, DMSO-d₆) δ7.93(m, 1H), 7.83-7.65 (m, 3H), 7.64-7.55 (m, 2H), 7.54-7.38 (m, 2H).

### EXAMPLE 12

### 2-((carboxycarbonyl)(cyclobutyl)amino)benzoic Acid

The desired product was prepared by substituting cyclobutylamine for cyclohexylamine in Example 22. MS (APCI(+)) m/e 264 (M+H)⁺; ¹H NMR (500 MHz, CD₃CN) δ8.00 (dd, 1H), 7.66 (dt, 1H), 7.53(dt, 1H), 7.32 (dd, 1H), 4.86-4.79 (m, 1H), 2.23-2.10 (m, 1H), 1.95-1.90 (m, 2H), 1.70-1.64 (m, 2H), 1.64-1.60 (m, 1H).

### EXAMPLE 13

### 2-((7-(benzyloxy)-1-naphthyl)(carboxycarbonyl)amino)benzoic Acid

### Example 13A

### 7-(benzyloxy)-1-naphthalenamine

A solution of 8-amino-2-naphthol (1.59 g, 10.0 mmol) in 1N KOH in methanol (10 mL) was concentrated, dissolved in N,N-dimethylformamide (10 mL), treated with benzyl bromide (1.2 mL, 10.1 mmol), stirred for two hours, poured into H₂O (50 mL), and extracted with ethyl acetate. The combined extracts were washed with water, dried (MgSO₄), filtered, and concentrated. The concentrate was purified by flash column chromatography on silica gel with 70:30 hexanes/ethyl acetate to provide the desired product.

### Example 13B

### 2-((7-(benzyloxy)-1-naphthyl)(carboxycarbonyl)amino)benzoic Acid

The desired product was prepared as a mixture of rotamers by substituting Example 13A for 1-aminonaphthalene in Example 1. MS (ESI(+)) m/e 442 (M+H); ¹H NMR (300 MHz, DMSO-d₆) δ8.06 (br s, 1H), 7.98-7.83(m, 3H), 7.69 (dd, 1H), 7.53-7.31 (m, 9H), 7.23(dd, 2H), 6.86 (dd, 1H), 5.23 (br s, 1H), 5.10 (d, 1H), 4.91 (d, 1H).

### EXAMPLE 14

### 2-((carboxycarbonyl)-2-(2-hydroxyethyl)anilino)benzoic Acid

The desired product was prepared by substituting 2-(2-aminophenyl)ethanol for 4-methoxyaniline in Example 3. MS (ESI(+)) m/e 330 (M+H)⁺; ¹H NMR (300 MHz, DMSO-d₆) δ7.98 and 7.85 (2 dd, 1H total ), 7.58-7.22 (m, 6H), 7.18 and 6.83(2 d, 1H total), 4.44 (t, 2H), 3.12-2.92 (m, 2H).

### EXAMPLE 15

### 2-((carboxycarbonyl)-2-methylanilino)benzoic Acid

The desired product was prepared by substituting ethyl (2E)-3-(2-bromophenyl)-2-propenoate and 2-methylaniline for Example 1A and 2-aminonaphthalene, respectively, in Example 1. MS(ESI(+)) m/e 300 (M+H)⁺; ¹H NMR (300 MHz, DMSO-d₆) δ7.55 (m, 1H), 7.42 (m, 2H), 7.30 (m, 2H), 7.15 (m, 3H), 2.24 (s, 3H).

### EXAMPLE 16

### 2-((carboxycarbonyl)(2-methyl-1H-indol-1-yl)amino)benzoic Acid

The desired product was prepared by substituting 2-methyl-1H-indol-1-amine for 4-methoxyaniline in Example 3. MS (ESI(+)) m/e 339 (M+H)⁺; ¹H NMR (300 MHz, DMSO-d₆) δ7.63-7.32 (m, 4H), 7.23-6.92 (m, 3H), 6.44-6.33 (m, 1H), 6.26 (s, 1H), 2.36 (s, 3H).

### EXAMPLE 17

### 2-((carboxycarbonyl)(7-hydroxy-1-naphthyl)amino)benzoic Acid

A solution of Example 13B (74 mg, 0.17 mmol) in dioxane (1 mL) at room temperature was treated with 10% Pd/C (10 mg) and 60% HClO₄ (1 drop), stirred under H₂ (1 atmosphere) for 4 hours, and filtered through diatomaceous earth (Celite®). The filter cake was washed with ethyl acetate, and the combined filtrates were concentrated. The concentrate was purified by reverse-phase HPLC with acetonitrile to 70:30 (0.1% aqueous trifluoroacetic acid)/acetonitrile to provide the desired product as a 5:3 mixture of rotamers. MS (ESI(+)) m/e 374 (M+Na)⁺; ¹H NMR (300 MHz, DMSO-d₆) δ10.04 (br s, 1H), 9.91 (br s, 1H), 7.87 (dd, 2H), 7.83(d, 2H), 7.79 (d, 1H), 7.61 (dd, 1H), 7.55-7.21 (m, 5H), 7.16 (dd, 1H), 7.12 (dd, 1H), 6.92 (d, 1H).

### EXAMPLE 18

### 2-((carboxycarbonyl)(7-((6-phenylhexyl)oxy)-1-naphthyl)amino)benzoic Acid

### Example 18A

### 1-(7-(benzyloxy)-1-naphthyl)-2(1H)-quinolinone

The desired product was prepared by substituting Example 13A for 1-aminonaphthalene in Example 1B.

### Example 18B

### 1-(7-hydroxy-1-naphthyl)-2(1H)-quinolinone

Example 18A (397 mg, 1.05 mmol) at room temperature was treated with 33% HBr in acetic acid (6 mL), stirred for 1 hour, poured into water (30 mL), and extracted with ethyl acetate. The combined extracts were washed sequentially with water, saturated NaHCO₃, and brine, dried (MgSO₄), filtered, treated with silica gel (5 mL), and concentrated under reduced pressure. The residue was purified by flash column chromatography on silica gel with 75:25 ethyl acetate/hexanes to provide the desired product.

### Example 18B

### 1-(7-((6-phenylhexyl)oxy)-1-naphthyl)-2(1H)-quinolinone

A solution of 6-phenyl-1-hexanol (31 mg, 0.17 mmol), Example 18A (50 mg, 0.17 mmol), and triphenylphosphine (46 mg, 0.17 mmol) in THF (1 mL) at room temperature was treated with diethylazodicarboxylate (30 mL, 0.19 mmol), stirred for 54 hours, and concentrated. The concentrate was dissolved in 1:1 ethyl acetate/hexanes, decanted, concentrated, and purified by flash column chromatography on silica gel with 1:1 ethyl acetate/hexanes to provide the desired product.

### Example 18C

### 2-((carboxycarbonyl)(7-((6-phenylhexyl)oxy)-1-naphthyl)amino)benzoic Acid

The desired product was prepared by substituting Example 18B for Example 1B in Example 1C. MS (ESI(+)) m/e 512 (M+H)⁺; ¹H NMR (300 MHz, DMSO-d₆) δ7.96-7.81 (m, 3H), 7.65 (d, 1H), 7.46-7.11 (m, 1OH), 6.86 (d, 1H), 3.99-3.81 (m, 1H), 3.82-3.75 (m, 1H), 2.56 (m, 2H), 1.56-1.43 (m, 4H), 1.42-1.20 (m, 4H).

### EXAMPLE 19

### 2-((1,1′-biphenyl)-2-yl(carboxycarbonyl)amino)benzoic Acid

The desired product was prepared as a mixture of rotamers by substituting (1,1′-biphenyl)-2-amine for 4-methoxyaniline in Example 3. MS (ESI(−)) m/e 360 (M−H)⁻; ¹H NMR (300 MHz, DMSO-d₆) δ7.75 and 7.54 (m, 1H total), 7.56-7.45 (m, 6H), 7.35-7.23(m, 4H), 7.15 (m, 1H), 6.68 and 6.44 (m, 1H total); Anal. Calcd for C₂₁H₁₅NO₅.1.81H₂O: C, 64.02; H, 4.76; N, 3.56. Found: C, 63.97; H, 4.41; N, 3.88.

### EXAMPLE 20

### 2-((1,1′-biphenyl)-4-yl(carboxycarbonyl)amino)benzoic Acid

The desired product was prepared by substituting (1,1′-biphenyl)-4-amine for 4-methoxyaniline in Example 3. MS (ESI(−)) m/e 360 (M−H)⁻; ¹H NMR (300 MHz, DMSO-d₆) d₆) δ7,62 (m, 4H), 7.53(m, 1H), 7.48-7.41 (m, 5H), 7.36 (m, 1H), 7.27 (d, 2H); Anal. Calcd for C₂₁H₁₅NO₅.3.22H₂O: C, 60.15; H, 5.15; N, 3.34. Found: C, 59.86; H, 4.43; N, 3.18.

### EXAMPLE 21

### 2-((carboxycarbonyl)(5,6,7,8-tetrahydro-1-naphthalenyl)amino)benzoic Acid

The desired product was prepared as a mixture of rotamers by substituting 5,6,7,8-tetrahydro-1-naphthalenamine for 1-aminonaphthalene in Example 1. MS (ESI(+) m/e 340 (M+H)⁺; ¹H NMR (500 MHz, DMSO-d₆) 8 7.92 (d, 1H), 7.81 (dd, 1H), 7.53(dt, 1H), 7.48 (dt, 1H), 7.41 (dt, 1H), 7.35 (dt, 1H), 7.23-7.04 (m, 3H), 6.97 (m, 1H), 6.84 (dd, 1H), 3.11 (td, 1H), 2.81 (br s, 1H), 2.75 (m, 1H), 2.63, (m, 1H), 2.10 (m, 1H), 1.83 (br s, 1H), 1.71-1.63(m, 2H), 1.62-1.51 (m, 1H).

### EXAMPLE 22

### 2-((carboxycarbonyl)(cyclohexyl)amino)benzoic Acid

### Example 22A

### Methyl 2-(cyclohexylamino)benzoate

A mixture of methyl-2-fluorobenzoate (158 mg, 1 mmol) and cyclohexylamine (210 mg, 2.05 mmol) in DMSO (1 mL) was heated to 100° C. for 18 hours, poured into water (10 mL), and extracted with diethyl ether. The combined extracts were washed with water and brine, dried (MgSO₄), filtered, and concentrated to provide the desired product.

### Example 22B

### Methyl 2-(cyclohexyl(ethoxy(oxo)acetyl)amino)benzoate

A solution of Example 22A (23mg, 0.1 mmol) in diethyl ether (1 mL) at room temperature was treated with pyridine (15 mL) and ethyl chloro(oxo)acetate (15 mL), stirred for 15 hours, diluted with 1N HCl (5 mL), and extracted with diethyl ether. The combined extracts were washed with brine, dried (MgSO₄), filtered, and concentrated. The concentrate was purified by flash column chromatography on silica gel with 70:30 ethyl acetate/hexanes to provide the desired product.

### Example 22C

### 2-((carboxycarbonyl)(cyclohexyl)amino)benzoic Acid

A mixture of Example 22B (18 mg, 0.054 mmol) in 1N NaOH in 5:1 ethanol:water (1 mL) at room temperature was stirred for 2.5 hours and concentrated. The concentrate was dissolved in water (5 mL), adjusted to pH <7 with 1N HCl (2 mL), and extracted with diethyl ether. The combined extracts were washed with brine, dried (MgSO₄), filtered, and concentrated provide the desired product. MS (ESI(+)) m/e 292 (M+H)⁺; ¹H NMR (300 MHz, CDCl₃) δ8.06 (dd, 1H), 7.64 (dt, 1H), 7.50 (dt, 1H), 7.32 (dd, 1H), 4.50-4.42 (m, 1H), 2.11-2.07 (m, 1H), 1.85-1.75 (m, 1H), 1.77-1.55 (m, 3H), 1.49-1.21 (m, 3H), 1.05-0.81 (m, 1H).

### EXAMPLE 23

### 2-((carboxycarbonyl)(2,3-dihydro-1,4-benzodioxin-6-yl)amino)benzoic Acid

The desired product was prepared by substituting 2,3-dihydro-1,4-benzodioxin-6-amine for 4-methoxyaniline in Example 3. MS (ESI(+)) m/e 344 (M+H)⁺; ¹H NMR (300 MHz, DMSO-d₆) δ7.52-7.38 (m, 4H), 6.78 (dd, 1H), 6.72 (d, 1H), 6.65 (dd, 1H), 4.19 (s, 4H).

### EXAMPLE 24

### 2-((carboxycarbonyl)(3-methylcyclohexyl)amino)benzoic Acid

The desired product was prepared as mixtures of diastereomers and rotamers by substituting 3-methylcyclohexylamine for cyclohexylamine in Example 22. MS (APCI(+)) m/e 323(M+NH₄)⁺; ¹H NMR (500 MHz, CD₃Cl) δ7.94 (m, 1H), 7.62 (m, 1H), 7.51 (m, 1H), 7.34 (m, 1H), 4.65-4.57 (m, 1H), 4.39-4.32 (m, 1H), 2.10-1.97 (m, 1H), 1.84-1.73 (m, 1H), 1.70-1.20 (m, 4H), 1.19-1.05 (m, 1H), 1.03-0.96 (m, 2H), 0.90 (d, 1H), 0.78 (d, 1H), 0.76-0.49 (m, 1H).

Following Schemes 1, 2, and 3 and the examples described above, Example 25-33 can be prepared:

### EXAMPLE 25

### 2-((carboxycarbonyl)-2-((E)-2-carboxyethenyl)anilino)benzoic Acid

### EXAMPLE 26

### 2-(2-((1E)-3-((tert-butoxycarbonyl)amino)-1-propenyl)(carboxycarbonyl)anilino)benzoic Acid

### EXAMPLE 27

### 2-((carboxycarbonyl)-2,3-dimethylanilino)benzoic Acid

### EXAMPLE 28

### 2-((carboxycarbonyl)-4-chloro-3-methylanilino)benzoic Acid

### EXAMPLE 29

### 2-(2-(aminocarbonyl)(carboxycarbonyl)anilino)benzoic Acid

### EXAMPLE 30

### 2-((7-(aminomethyl)-5,6,7,8-tetrahydro-1-naphthalenyl)(carboxycarbonyl)amino)benzoic Acid

### EXAMPLE 31

### 2-((6-(aminomethyl)-5,6,7,8-tetrahydro-1-naphthalenyl)(carboxycarbonyl)amino)benzoic Acid

### EXAMPLE 32

### 2-((carboxycarbonyl)(5-(2,3-diamino-3-oxopropyl)-5,6,7,8-tetrahydro-1-naphthalenyl)amino)benzoic Acid

### EXAMPLE 33

### 2-((carboxycarbonyl)-4-(2,3-diamino-3-oxopropyl)anilino)benzoic Acid

### EXAMPLE 34

### 2-[(carboxycarbonyl)(2-hydroxy-1-naphthyl)amino]benzoic Acid

### Example 34A

### Toluene-4-sulfonic acid 1I-amino-naphthalen-2-yl ester

A mixture of I1-amino-2-naphthol hydrochloride (3 g, 15 mmol), p-toluenesulfonyl chloride (2.9 g, 15 mmol) and triethylamine (4.3 mL, 31 mmol)in dichloromethane (150 mL) was stirred at ambient temperature for 18 hours, diluted with additional methylene chloride, washed with water, brine, dried (MgSO₄), filtered, and concentrated under reduced pressure. The product was purified via silica gel chromatography, eluting with 3:1 methylene chloride:hexanes to provide the titled compound (940 mg, 20%).

### Example 34B

### 2-[2-(toluene-4-sulfonyloxy)-naphthalen-1-ylamino]-benzoic Acid

The desired product was prepared according to the method described in Example 48E using diphenyliodonium-2-carboxylate and toluene-4-sulfonic acid 1-amino-naphthalen-2-yl ester.

### Example 34C

2-[(2-hydroxy-naphthalen-1-yl)-oxalyl-amino]-benzoic Acid

To a solution of 2-[2-(toluene-4-sulfonyloxy)-naphthalen-1-ylamino]-benzoic acid (326 mg, 0.752 mmol) and N,N-diisopropylethylamine (0.7 mL, 3.8 mmol) in toluene (10 mL) was added ethyl oxalyl chloride (0.4 mL, 3.8 mmol). The reaction was heated to reflux for 18 hours, cooled and extracted with 1 M NaOH. The aqueous layer was extracted with diethyl ether, the pH adjusted to 2 by the addition of 1M HCl, and further extracted with ethyl acetate. The combined ethyl acetate layers were dried (MgSO₄), filtered, concentrated under reduced pressure and purified via silica gel chromatography eluting with 1:1 hexanes:ethyl acetate to provide the oxalamide ethyl ester 395 mg (99%). The ester was taken up in a mixture of 1.39 M NaOH (8 mL) in 20% aqueous ethanol (20 mL) and stirred at ambient temperature for 16 hours. The solvents were removed under reduced pressure, the residue was taken up in water, acidified to a pH of 2 with 1M HCl, and extracted with ethyl acetate. The combined ethyl acetate was dried (MgSO₄), filtered, and concentrated under reduced pressure to provide the title compound (253 mg (96%). ¹H NMR (300 MHz, d₆-DMSO) δ8.16 (d, 1H, J=8.1), 7.89-7.80 (m, 2H), 7.49-7.44 (m, 1H), 7.39-7.24 (m, 4H), 7.11 (d, 1H, J=8.4), 6.84 (dd, 1H, J=1.0, 8.2); MS (ESI) m/z=350 (M−H), 369 (M+NH4⁺), 374 (M+Na⁺).

### EXAMPLE 35

### N-acetyl-4-[(carboxycarbonyl)(2-carboxyphenyl)amino]-N-pentyl-1-naphthylalaninamide

### Example 35A

### 2-acetylamino-3-(4-amino-naphthalen-1-yl)-acrylic acid methyl Ester

To a mixture of 4-bromo-1-naphthylamine (2.5 g, 11.3 mmol), Pd(OAc)₂ (140 mg, 0.63 mmol), P(o-tolyl)₃(570 mg, 1.87 mmol) in anhydrous N,N-dimethylformamide (10 mL) in a pressure tube was added methyl 2-acetamidoacrylate (2.1 g, 14.7 mmol) and triethylamine (5.3 mL, 37.5 mmol). The mixture was flushed with nitrogen for 3 min before it was sealed and heated to 110° C. for 4 hours. The reaction mixture was cool to ambient temperature, partitioned between ethyl acetate and water. The aqueous layer was extracted once with ethyl acetate and the combined organic layers were washed with brine, dried (Na₂SO₄), filtered, concentrated under reduced pressure and purified on a silica gel to provide the titled compound (2.5 g, 76%).

### Example 35B

### 2-acetylamino-3-(4-amino-naphthalen-1-yl)-propionic Acid Methyl Ester

To a solution containing 2-acetylamino-3-(4-amino-naphthalen-1-yl)-acrylic acid methyl ester (2.5 g, 8.8 mmol) in ethyl acetate/methanol (50 mL, 1:1 by volume) under N₂ atmosphere was added Pd/C (10%, 250 mg). The reaction flask was capped with a H₂ balloon and heated to 60° C. for 18 hours. The mixture was filtered through celite, the filtration bed washed with ethyl acetate/methanol (2×25 mL, 1:1). The combined filtrate was concentrated under reduced pressure to provide the titled compound (2.5 g, 100%).

### Example 35C

### 2-acetylamino-3-(4-amino-naphthalen-1-yl)-propionic Acid

To a solution containing 2-acetylamino-3-(4-amino-naphthalen-1-yl)-propionic acid methyl ester (2.5 g, 8.8 mmol) in methanol (50 mL) was added dropwise 3N NaOH (4.75 mL, 14.3 mmol) and stirred for 3 hours. The mixture was reduced in volume under reduced pressure, acidified to pH 4.5 with 3N HCl. The mixture was concentrated to dryness under reduced pressure, taken up in methanol/dichloromethane (10%, 25 mL), filtered through celite. The filter cake was washed with additional methanol/dichloromethane (10%, 25 mL). The filtrate was concentrated under reduced pressure to provide the titled compound (1.75 g, 74%).

### Example 35D

### 2-acetylamino-3-(4-amino-naphthalen-1-yl)-N-pentyl-propionamide

A solution of 2-acetylamino-3-(4-amino-naphthalen-1-yl)-propionic acid (500 mg, 1.84 mmol), 1-[3-(dimethylamino)propyl]-3-ethylcarbodiimide hydrochloride (493 mg, 2.57 mmol), 1-hydroxybenzotriazole hydrate (360 mg, 2.21 mmol), amylamine (320 μL, 2.75 mmol) in anhydrous N,N-dimethylformamide (10 mL) was adjusted to the pH˜6 by the addition of triethylamine and stirred for 5 hours. The reaction was diluted with water `and extracted with ethyl acetate (3×15 mL). The combined ethyl acetate was washed with water, brine, dried (Na₂SO₄), concentrated under reduced pressure and purified on a silica gel to provide the titled compound (552 mg, 1.62 mmol, 88%).

### Example 35E

### 2-[4-(2-acetylamino-2-pentylcarbamoyl-ethyl)-naphthalen-1-ylamino]-benzoic Acid

To a stirred suspension of 2-acetylamino-3-(4-amino-naphthalen-1-yl)-N-pentyl-propionamide (552 mg, 1.62 mmol) and diphenyliodonium-2-carboxylate monohydrate 10 (580 mg, 1.78 mmol) in N,N-dimethylformamide (10 mL) was added anhydrous Cu(OAc)₂ (14.6 mg, 0.081 mmol). The resulting mixture was heated to 95° C. for 1.5 hour. The reaction mixture concentrated under reduced pressure after which the N,N-dimethylformamide was distilled out. The residue was further concentrated to a constant weight on an oil pump (790 mg).

### Example 35F

### 2-{[4-(2-acetylamino-2-pentylcarbamoyl-ethyl)-naphthalen-1-yl]-ethoxyoxalyl-amino}-benzoic Acid

A stirred solution of 2-[4-(2-acetylamino-2-pentylcarbamoyl-ethyl)-naphthalen-1-ylamino]-benzoic acid (790 mg, 1.71 mmol) and triethylamine (680 μL, 5.13 mmol) in dichloromethane (10 mL) was cooled to 0° C., treated slowly with ethyl oxalyl chloride (452 μL, 4.04 mmol) over 30 min, warmed to room temperature, stirred for 16 hours, treated with 1N HCl (4 mL), and extracted with dichloromethane (2×20 mL). The combined extracts were dried (Na₂SO₄), filtered, and concentrated. The concentrate was purified by a Gilson preparative HPLC to give 291 mg of the acylated product (0.52 mmol, 31% over two steps) as a light yellow foam.

### Example 35G

### N-acetyl-4-[(carboxycarbonyl)(2-carboxyphenyl)amino]-N-pentyl-1-naphthylalaninamide

A solution of 2-{[4-(2-acetylamino-2-pentylcarbamoyl-ethyl)-naphthalen-1-yl]-ethoxyoxalyl-amino}-benzoic acid (291 mg, 0.52 mmol) in methanol (5 mL) at room temperature was treated with 1N NaOH (1.3 mL, 1.3 mmol), stirred for 2 hours, treated with 1N HCl (2 mL), and purified by reverse-phase HPLC with acetonitrile to 95:5 (0.1% aqueous trifluoroacetic acid)/acetonitrile to provide 200 mg of the desired product (0.37 mmol, 72%). MS (ESI+) m/e 534 (M+H)⁺; ¹H NMR (300 MHz, DMSO-d₆) (A mixture of rotamers) δ8.45-8.15 (m, 3H), 8.10-7.80 (m, 3H), 7.68 (brs, 1H), 7.64-7.27 (m, 4H), 7.18 (brs, 1H), 6.86-6.77 (m, 1H), 4.58 (q, J=7.4 Hz, 1H), 3.67-2.82 (m, 6H), 1.85-1.70 (three s, 3H in total), 1.37-1.00 (m, 4H), 0.92-0.69 (m, 3H).

### EXAMPLE 36

### N-acetyl-4-[(carboxycarbonyl)(2-carboxyphenyl)amino]-N-pentyl-3-(2-hydroxyethane)-phenylalaninamide

### Example 36A

### 2-(2-amino-5 -bromo-phenyl)-ethanol

To a solution of 2-aminophenethyl alcohol (10.0g, 72.9 mmol) in acetic acid (60 mL) at 10° C. was added Br₂ (3.8 mL, 72.9 mmol) in acetic acid (5 mL). Additional acetic acid (30 mL) was added and the reaction was stirred for 1 hour. The mixture was filtered and the filter cake washed with diethyl ether. The solid was then partitioned between ethyl acetate and aqueous 3N NaOH. The organic layer was washed with brine, dried (Na₂SO₄), filtered and concentrated under reduced pressure to provide the titled compound (15.8 g).

### Example 36B

### 4-bromo-2-(1-methyl-1-trimethylsilanyl-ethoxymethyl)-phenylamine

To a solution of 2-(2-amino-5-bromo-phenyl)-ethanol (15.8 g, 72.8 mmol) in anhydrous N,N-dimethylformamide (50 mL) was added imidazole (6.0 g, 88.1 mmol) and tert-butyl dimethylsilyl chloride (12.0 g, 79.6 mmol) sequentially. The resulting mixture was stirred at ambient temperature for 1.5 hour, partitioned between water and ethyl acetate. The organic layer was washed with water, brine, dried (Na₂SO₄), filtered, concentrated under reduced pressure and purified on silica gel with 10-15% ethyl acetate/hexanes to provide the titled compound (15.0 g, 62.3%). MS (ESI+) m/e 330, 332 (M+H)⁺.

### Example 36C

### 2-acetylamino-3-[4-amino-3-(2-hydroxy-ethyl)-phenyl]-propionic Acid

The titled compound was prepared according to the procedure described in Example 79 B-C, substituting 4-bromo-2-(1-methyl-1-trimethylsilanyl-ethoxymethyl)-phenylamine for the 4-bromo-2-ethylalanine. The tert butyldimethyl silyl protecting group came off during the hydrogenation step as described in Example 79C. MS (ESI+) m/e 381 (M+H)⁺.

### Example 36D

### N-acetyl-4-[(carboxycarbonyl)(2-carboxyphenyl)amino]-N-pentyl-3-(2-hydroxyethane)-phenylalaninamide

The titled compound was prepared according to the procedure described in 35 C-G by substituting 2-acetylamino-3-[4-amino-3-(2-hydroxy-ethyl)-phenyl]-propionic acid for 2-acetylamino-3-(4-amino-naphthalen-1-yl)-propionic acid. MS (ESI+) m/e 528 (M+H)⁺, 545 (M+NH₄); ¹H NMR (300 MHz, DMSO-d₆) (A mixture of rotamers) δ8.14-7.58 (m, 3H), 7.52-6.85 (m, 6H), 4.80-4.30 (m, 3H), 3.60-3.79 (m, 2H), 3.04-2.64 (m, 6H), 1.82-1.73 (multiple s, 3H in total), 1.40-1.10 (m, 4H), 0.84 (t, J=7.4 Hz, 3H).

### EXAMPLE 37

### 4-[(carboxycarbonyl)(2-carboxyphenyl)amino]-6-{[N-acetyl-3-(1-naphthyl)alanyl]amino}hexanoic Acid

The titled compound was prepared according to the procedure described in 35 D-G by substituting 6-amino-hexanoic acid methyl ester HCl salt for amylamine. MS (ESI+) m/e 578 (M+H)⁺; ¹H NMR (300 MHz, DMSO-d₆) (A mixture of rotamers) δ8.45-8.15 (m, 3H), 8.10-7.80 (m, 3H), 7.68 (brs, 1H), 7.65-7.28 (m, 4H), 7.17 (brs, 1H), 6.78-6.85 (m, 1H), 4.65-4.50 (m, 1H), 2.85-3.60 (m, 4H), 2.14 (q, J=7.1Hz, 2H), 1.83-1.70 (multiple s, 3H in total), 1.56-1.01 (m, 6H).

### EXAMPLE 38

### 4-[(carboxycarbonyl)(2-carboxyphenyl)amino]-3-[(1E)-3-amino-3-oxo-1-propenyl]-N-(tert-butoxycarbonyl)-N-pentyl-L-phenylalaninamide

### Example 38A

### [2-(4-amino-phenyl)-1-pentylcarbamoyl-ethyl]-carbamic acid tert-butyl Ester

The titled compound was prepared according to the procedure described in Example 35 D substituting N-boc p-amino phenylalanine for 2-acetylamino-3-(4-amino-naphthalen-1-yl)-propionic acid.

### Example 38B

### [2-(4-amino-3-iodo-phenyl)-1-pentylcarbamoyl-ethyl]-carbamic acid tert-butyl Ester

To a stirred solution of [2-(4-amino-phenyl)-1-pentylcarbamoyl-ethyl]-carbamic acid tert-butyl ester (1.2 g, 3.4 mmol) in acetic acid (5 mL) was added NaI (0.59 g, 3.9 mmol) followed by the addition of chloramines-T trihydrate (1.1 g, 3.9 mmol). The solution was stirred for one hour, concentrated under reduced pressure, diluted with aqueous Na₂S₂O₄ solution, and partitioned between ethyl acetate and aqueous NaHCO₃. The organic layer was washed with brine, dried (Na₂SO₄), filtered, concentrated under reduced pressure and purified on silica gel eluting with 20% ethyl acetate/hexanes to provide the titled compound 0.78 g (46%). MS (ESI+) m/e 476 (M+H)⁺.

### Example 38C

### 2-[4-(2-tert-butoxycarbonylamino-2-pentylcarbamoyl-ethyl)-2-iodo-phenylamino]-benzoic Acid

To a stirred suspension of [2-(4-amino-3-iodo-phenyl)-1-pentylcarbamoyl-ethyl]-carbamic acid tert-butyl ester (222 mg, 0.47 mmol) and diphenyliodonium-2-carboxylate monohydrate (168 mg, 0.49 mmol) in N,N-dimethylformamide (5 mL) was added anhydrous Cu(OAc)₂ (7.3 mg, 0.040 mmol). The resulting mixture was heated to 95° C. for 1.5 hour. The reaction mixture concentrated under reduced pressure after which the N,N-dimethylformamide was distilled out. The residue was further concentrated to a constant weight on an oil pump to give the titled compound as a light brown solid (306 mg). MS (ESI+) m/e 534 (M+H)⁺.

### Example 38D

### 2-[4-(2-tert-Butoxycarbonylamino-2-pentylcarbamoyl-ethyl)-2-[(1E)-3-amino-3-oxo-1-propenyl]-phenylamino]-benzoic Acid

To a mixture of 2-[4-(2-tert-butoxycarbonylamino-2-pentylcarbamoyl-ethyl)-2-iodo-phenylamino]-benzoic acid (306 mg, 0.51 mmol), Pd(OAc)₂ (6 mg, 0.026 mmol), P(o-tolyl)₃ (23 mg, 0.80 mmol) in anhydrous N,N-dimethylformamide (10 mL) in a pressure tube was added acrylamide (66 mg, 0.93 mmol) and triethylamine (0.25 mL, 1.79 mmol). The mixture was flushed with nitrogen for 3 minutes before it was sealed and heated to 90° C. for 16 hours. The reaction mixture was cool to ambient temperature, solvent was removed on a SpeedVac. The residue was taken up in methanol and purified on a Gilson preparative HPLC using acetonitrile: 0.3 mM aqueous NH₄OAc to provide the titled compound (153 mg, 0.28 mmol). MS (ESI+) m/e 539 (M+H)⁺.

### Example 38E

### 4-[(carboxycarbonyl)(2-carboxyphenyl)amino]-3-[(1E)-3-amino-3-oxo-1-propenyl]-N-(tert-butoxycarbonyl)-N-pentyl-L-phenylalaninamide

A stirred solution of 2-[4-(2-tert-Butoxycarbonylamino-2-pentylcarbamoyl-ethyl)-2-[(1E)-3-amino-3-oxo-1-propenyl]-phenylamino]-benzoic acid (153 mg, 0.28 mmol) and triethylamine (119 μL, 0.85 mmol) in dichloromethane (3 mL) was cooled to 0° C., treated slowly with ethyl oxalyl chloride (70 μL, 0.63 mmol) over 30 minutes, warmed to room temperature, stirred for 16 hours, treated with aqueous 1N HCl (4 mL), and extracted with dichloromethane (2×20 mL). The combined extracts were dried (Na₂SO₄), filtered, and concentrated. The concentrate was purified by a Gilson preparative HPLC. The residue (89 mg, 0.14 mmol) in methanol (2 mL) at room temperature was treated with aqueous 1N NaOH (0.42 mL, 0.42 mmol), stirred for 2 hours, treated with aqueous 1N HCl (1 mL), and purified by reverse-phase HPLC eluting with acetonitrile: 3 mM aqueous NH₄OAc) to provide the titled compound (65 mg, 76%). MS (ESI+) m/e 628 (M+NH₄)⁺; ¹H NMR (300 MHz, DMSO-d₆) (A mixture of rotamers) δ7.81 (dd, J=2.1, 8.4 Hz, 1H), 7.63(d, J=8.4 Hz, 2H), 7.72-6.83(m, 7H), 6.79 (t, J=7.5 Hz, 1H), 6.52 (d, J=15.8 Hz, 1H), 4.19-4.01 (m, 1H), 3.10-2.82 (m, 4H), 1.31-1.10 (m, 15H), 0.83(t, J=7.2 Hz, 3H).

### EXAMPLE 39

### N-acetyl-4-[(carboxycarbonyl)(2-carboxyphenyl)amino]-3-isopropyl-N-pentylphenylalaninamide

### Example 39A

### 2-acetylamino-3-(4-amino-3-isopropyl-phenyl)-propionic Acid

The titled compound was prepared according to the procedure described in Example 35 A-C, substituting 4-bromo-2-isopropylaniline for the 4-bromo-1-naphthylamine in Example 35A.

### Example 39B

### N-acetyl-4-[(carboxycarbonyl)(2-carboxyphenyl)amino]-3-isopropyl-N-pentylphenylalaninamide

The titled compound was prepared according to the procedure described in Example 35 D-G, substituting 2-acetylamino-3-(4-amino-3-isopropyl-phenyl)-propionic acid from Example 39A for 2-acetylamino-3-(4-amino-naphthalen-1-yl)-propionic acid in Example 35 D. MS (ESI(+)) m/e 526 (M+H)⁺, 543 (M+NH₄)+; 1H NMR (500 MHz, DMSO-d₆) A mixture of rotamers: δ7.90-8.16 (m, 2H), 6.73-7.60 (m, 4H), 4.40-4.52 (m, c.a. 0.6 H), 2.7-3.3 (m, c.a. 5.4H), 1.77 and 1.74 (s, 3H), 1.1-1.41 (m, 11H), 0.62-0.90 (m, 3H).

### EXAMPLE 40

### 4-[(carboxycarbonyl)(2-carboxyphenyl)amino]-6-{[N-acetyl-3-(1-piperidinyl)phenylalanyl]amino}hexanoic Acid

### Example 40A

### 2-acetylamino-3-(4-amino-3-piperidin-1-yl-phenyl)-propionic Acid

The desired product was prepared according to the procedure described in Example 35 A-C, substituting 4-bromo-2-piperidin-1-yl-aniline for. 4-bromo-1-naphthylamine in Example 35A.

### Example 40B

### 4-[(carboxycarbonyl)(2-carboxyphenyl)amino]-6-{[N-acetyl-3-(1-piperidinyl)phenylalanyl]amino}hexanoic Acid

The desired product was prepared according to the procedure described in Example 35 D-G, substituting 2-acetylamino-3-(4-amino-3-piperidin-1-yl-phenyl)-propionic acid for 2-acetylamino-3-(4-amino-naphthalen-1-yl)-propionic acid and substituting 6-amino-hexanoic acid methyl ester HCl salt for amylamine in Example 35D. MS (ESI(+)) m/e 611 (M+H)⁺, 633(M+Na)⁺; ¹H NMR (500 MHz, DMSO-d₆) a mixture of rotamers: δ7.90-8.16 (m, 2H), 6.73-7.60 (m, 4H), 4.40-4.52 (br m, c.a. 0.6 H), 2.7-3.3 (m, c.a. 8.4H), 1.77 and 1.74 (s, 3H), 1.19-1.62 (m, 18H).

### EXAMPLE 41

### 2-{(carboxycarbonyl) [2-(3-methyl-1-piperidinyl)phenyl]amino}benzoic Acid

### Example 41A

### 3-Methyl-1-(2-nitro-phenyl)-piperidine

A solution of 3-methylpiperidine (0.848 mL, 7.22 mmol), 2-chloronitrobenzene (1.04 g, 6.57 mmol) and diisopropylethylamine (1.26 mL, 7.22 mmol) in DMSO (5 mL) were heated to 90-95° C. under N₂ overnight. The mixture was cooled to ambient temperature then partioned between a mixture of ethyl acetate:hexane (1:1) and water (1:1, 75 mL total). The organic phase separated, dried (Na₂SO₄), filtered, concentrated under reduced pressure and purified by flash column chromatography (5% to 8% ethyl acetate/hexane) to give 2-(3-methyl-piperidin-1-yl)-nitrobenzene (1.03 g).

### Example 41B

### 2-(3-methyl-piperidin-1-yl)-phenylamine

A solution of 2-(3-methyl-piperidin-1-yl)-nitrobenzene (1.00 g, 4.54 mmol) and 10% Pd/C (0.24 g, 0.227 mmol) in ethanol/ethyl acetate (7/3 mL) was stirred under an atmosphere of hydrogen for 24 hours. The mixture was filtered through Celite and the solids were washed thoroughly with ethyl acetate. The combined filtrate was concentrated to under reduced pressure to provide the titled compound (0.86 g).

### Example 41C

### 2-{(carboxycarbonyl)[2-(3-methyl-1-piperidinyl)phenyl]amino}benzoic Acid

The desired product was prepared according to the procedure described in Example 3 by substituting 2-(3-methyl-piperidin-1-yl)-phenylamine for 4-methoxyaniline. MS (ESI(+)) m/e 383 (M+H)⁺; ¹H NMR (500 MHz, DMSO-d₆) a mixture of rotamers: δ7.80 (m, 1H), 7.55 (m, 1H), 7.22-7.38 (m, 3H), 6.9-7.18 (m, 3H), 2.6-3.3 (m, 3H), 2.1-2.4 (m, 2H), 1.2-1.8 (m, 4H), 0.8-1.0 (m, 4H).

### EXAMPLE 42

### 2-{(carboxycarbonyl)[5-hydroxy-2-(1-piperidinyl)phenyl]amino}benzoic Acid

### Example 42A

### 1-(4-Methoxymethoxy-2-nitro-phenyl)-piperidine

To a mixture of 4-chloro-3-nitrophenol (1.0 g, 5.8 mmol) and K₂CO₃ (1.6 g, 12 mmol) in N,N-dimethylformamide (5 mL) at ambient temperature was slowly added chloromethyl methylether. After 10 min, piperidine (1.2 mL, 5.8 mmol) was added, the mixture was heated to 80° C. for 2 days, cooled to ambient temperature, partitioned between ethyl acetate and water. The organic layer was washed with brine, dried (Na₂SO₄), filtered, concentrated under reduced pressure and purified on a silica gel flash column eluting with 15% EtOAc/hexanes to provide the titled compound (0.98 g, 64%).

### Example 42B

### 2-[(5-methoxymethoxy-2-piperidin-1-yl-phenyl)-oxalyl-amino]-benzoic Acid

The titled compound was prepared according to the procedure described in Example 41B-C, substituting the 1-(4-methoxymethoxy-2-nitro-phenyl)-piperidine for 3-methyl-1-(2-nitro-phenyl)-piperidine in Example 41 B. MS (ESI+) m/e 429 (M+H)⁺.

### Example 42C

### 2-{(carboxycarbonyl)[5-hydroxy-2-(1-piperidinyl)phenyl]amino}benzoic Acid

A solution containing 2-[(5-methoxymethoxy-2-piperidin-1-yl-phenyl)-oxalyl-amino]-benzoic acid (25 mg, 0.058 mmol) in methylene chloride/trifluoroacetic acid (2 mL, 1:1, v:v) was stirred at ambient temperature for 2 hours, concentrated under reduced pressure, taken up in diethyl ether and filtered. The filter cake was dried to constant weigh to provide the titled compound (15 mg, 67%). MS (ESI+) m/e 385 (M+H)⁺; ¹H NMR (300 MHz, DMSO-d₆) (A mixture of rotamers) δ9.52 (brs, 1H), 7.79 (dd, J=2.1, 8.4 Hz, 1H), 7.53(td, J=2.1, 8.4 Hz, 1H), 7.37 (td, J=0.9, 7.65 Hz, 1H), 7.20 (brd, J=7.5 Hz, 1H), 6.99 (brm, 1H), 6.70 (brd, J=7.5 Hz, 1H), 6.55 (brm, 1H), 2.83-2.56 (m, 4H), 1.69-1.30 (m, 6H).

### EXAMPLE 43

### 4-[(carboxycarbonyl)(2-carboxyphenyl)amino]-3-[(1E)-3-amino-3-oxo-1-propenyl]-N-(methylsulfonyl)-N-pentyl-L-phenylalaninamide

### Example 43A

### 2-amino-3-(4-nitro-phenyl)-N-pentyl-propionamide

A solution containing [2-(4-nitro-phenyl)-1-pentylcarbamoyl-ethyl]-carbamic acid tert-butyl ester from Example 50A (500 mg, 1.3 mmol) in methylene chloride/trifluoroacetic acid (10 mL, 1:1, v:v) was stirred at ambient temperature for 2 hours, concentrated under reduced pressure to provide the titled compound as its trifluoroacetic acid salt.

### Example 43B

### 2-methane sulfonylamino-3-(4-nitro-phenyl)-N-pentyl-propionamide

To a solution of 2-amino-3-(4-nitro-phenyl)-N-pentyl-propionamide (368 mg, 1.3 mmol) in methylene chloride (10 mL) at 0° C. was added triethylamine (500 μL, 3.6 mmol), followed by methane sulfonyl chloride (122 μL, 1.6 mmol). After stirring at ambient temperature for one hour, the reaction mixture was partitioned between ethyl acetate and aqueous 3N HCl. The organic layer was washed with aqueous NaHCO₃, brine, dried (Na₂SO₄), filtered and concentrated under reduced pressure to provide the titled compound.

### Example 43C

### 3-(4-amino-phenyl)-2-methanesulfonylamino-N-pentyl-propionamide

The titled compound was prepared according to the procedure described for Example 50B, substituting 2-methanesulfonylamino-3-(4-nitro-phenyl)-N-pentyl-propionamide for [2-(4-nitro-phenyl)-1-pentylcarbamoyl-ethyl]-carbamic acid tert-butyl ester.

### Example 43D

### 3-(4-amino-3-iodo-phenyl)-2-methanesulfonylamino-N-pentyl-propionamide

The titled compound was prepared according to the procedure described for Example 38B, substituting 3-(4-amino-phenyl)-2-methanesulfonylamino-N-pentyl-propionamide for [2-(4-amino-3-iodo-phenyl)-1-pentylcarbamoyl-ethyl]-carbamic acid tert-butyl ester.

### Example 43E

### 4-[(carboxycarbonyl)(2-carboxyphenyl)amino1-3-[(1E)-3-amino-3-oxo-1-propenyl]-N-(methylsulfonyl)-N-pentyl-L-phenylalaninamide

The titled compound was prepared according to the procedure described for Example 38 C-E, substituting 3-(4-amino-3-iodo-phenyl)-2-methanesulfonylamino-N-pentyl-propionamide for 2-[4-(2-tert-butoxycarbonylamino-2-pentylcarbamoyl-ethyl)-2-iodo-phenylamino]-benzoic acid. MS (ESI+) m/e 589 (M+H)⁺, 606 (M+NH₄); ¹H NMR (300 MHz, DMSO-d₆) (A mixture of rotamers) δ8.05-7.92 (m, 1H), 7.79 (d, J=15.6 Hz, 1H), 7.86-7.23 (m, 7H), 7.17 (d, J=7.8 Hz, 1H), 7.05 and 6.79 (d, J=8.4 Hz, 1H in total), 6.74-6.53 (three sets of d, J=15.6 Hz, 1H in total), 4.38 (overlapping m, 1H), 3.10-2.69 (m, 4H), 2.43(s, 3H), 1.11-1.42 (m, 6H), 0.90-0.77 (m, 3H).

### EXAMPLE 44

### 4-[(carboxycarbonyl)(2-carboxyphenyl)amino]-3-(3-amino-3-oxopropyl)-N-[(isopropylamino)carbonyl]-N-pentyl-L-phenylalaninamide

A solution containing 4-[(carboxycarbonyl)(2-carboxyphenyl)amino]-3-[(1E)-3-amino-3-oxo-1-propenyl]-N-[(isopropylamino)carbonyl]-N-pentyl-L-phenylalaninamide (25 mg, 43 mmol) and 10% Pd/C (10 mg) in methanol/ethyl acetate (2 mL, 1:1/v:v) was stirred under an atmosphere of hydrogen for 2 hours. The mixture was filtered through celite and concentrated under reduced pressure to provide the titled compound (20 mg, 80%). MS (ESI+) m/e 598 (M+H)⁺.

### EXAMPLE 45

### 4-[(carboxycarbonyl)(2-carboxyphenyl)amino]-3-(1E)-3-amino-3-oxo-1-propenyl]-N-[(isopropylamino)carbonyl]-N-pentyl-L-phenylalaninamide

### Example 45A

### 2-(3-isopropyl-ureido)-3-(4-nitro-phenyl)-N-pentyl-propionamide

The titled compound was prepared according to the procedure described for Example 43B, substituting i-propyl isocyanate for the methane sulfonyl chloride.

### Example 45B

### 4-amino-N-[(isopropylamino)carbonyl]-N-pentyl-L-phenylalaninamide

The titled compound was prepared according to the procedure described for Example 50B, substituting 2-(3-isopropyl-ureido)-3-(4-nitro-phenyl)-N-pentyl-propionamide for [2-(4-nitro-phenyl)-1-pentylcarbamoyl-ethyl]-carbamic acid tert-butyl ester.

### Example 45C

### 4-[(carboxycarbonyl)(2-carboxyphenyl)amino]-3-[(1E)-3-amino-3-oxo-1-propenyl]-N-[(isopropylamino)carbonyl]-N-pentyl-L-phenylalaninamide

The titled compound was prepared according to the procedure described for Example 38B-E substituting 4-amino-N-[(isopropylamino)carbonyl]-N-pentyl-L-phenylalaninamide for [2-(4-amino-phenyl)-1-pentylcarbamoyl-ethyl]-carbamic acid tert-butyl ester. MS (ESI+) m/e 596 (M+H)⁺; ¹H NMR (300 MHz, DMSO-d₆) (A mixture of rotamers) δ8.15-7.92 (m, 1H), 7.79 (d, J=15.6 Hz, 1H), 7.86-7.23 (m, 7H), 7.17 (d, J=7.8 Hz, 1H), 7.05 and 6.79 (d, J=8.4 Hz, 1H in total), 6.74-6.53 (three sets of d, J=15.6 Hz, 1H in total), 4.35 (overlapping m, 1H), 3.62 (qd, J=12.6, 6.6 Hz, 1H), 3.10-2.69 (m, 4H), 1.30 (t, J=6.6 Hz, 6H), 1.11-1.42 (m, 6H), 0.90-0.77 (m, 3H).

### Example 46 i

### 2-((carboxycarbonyl){2-[4-(hydroxymethyl)-1-piperidinyl]phenyl}amino)benzoic Acid

### Example 46 A

### [1-(2-amino-phenyl)-piperidin-4-yl]-methanol

The titled compound was prepared according to the procedure described in Example 41 by substituting 3-methylpiperidine with piperidin-4-yl-methanol.

### Example 46B

### 2-((carboxycarbonyl){2-[4-(hydroxymethyl)-1-piperidinyl]phenyl}amino)benzoic Acid

The desired product was prepared according to the procedure described in Example 3 by substituting [1-(2-amino-phenyl)-piperidin-4-yl]-methanol for 4-methoxyaniline. MS (ESI(+)) m/e 399 (M+H)⁺; ¹H NMR (500 MHz, DMSO-d₆) a mixture of rotamers: δ7.80 (m, 1H), 7.55 (m, 1H), 7.22-7.38 (m, 3H), 6.9-7.18 (m, 3H), 2.6-3.3(m, 2H), 2.1-2.4 (m, 2H), 1.5-1.8 (m, 4H),1.25 (t, 1H), 1.05 (t, 1H), 0.9-1.0 (m, 2H).

### EXAMPLE 47

### N-acetyl-4-[(carboxycarbonyl)(2-carboxyphenyl)amino]-N-pentyl-4-(1-piperidinyl)phenylalaninamide

The desired product was prepared according to the procedure described in Example 35 D-G, substituting 2-acetylamino-3-(4-amino-3-piperidin-1-yl-phenyl)-propionic acid from Example 40A for 2-acetylamino-3-(4-amino-naphthalen-1-yl)-propionic acid in Example 35D. MS (ESI(+)) m/e 567 (M+H)⁺; 1H NMR (500 MHz, DMSO-d₆) a mixture of rotamers: δ6.9-8.0 (m, 7H), 4.4 (br m, 1H), 3.3-4.1 (br m, 6H), 2.6-3.0 (m, 6H), 1.1-1.78 (m, I 1H), 0.82 (m, 2H).

### EXAMPLE 48

### N-acetyl-4-[(carboxycarbonyl)(2-carboxyphenyl)amino]-3-ethylphenylalanyl-N-methyl-4-nitro-L-phenylalaninamide

### Example 48A

### (S)-N-boc-4-nitrophenylalanine

To a solution of (S)-4-nitrophenylalanine (2.00 g, 9.51 mmol) in aqueous 2M NaOH (10 mL) and THF (5 mL), was added di-tertbutyldicarbonate (2.00 g, 9.17 mmol) in THF (5 mL). The reaction was stirred at ambient temperature for 1 hour, diluted with H₂O (25 mL) and 1M HCl (25 mL), extracted with ether (3×25 mL). The combined ether layers were extracted with brine (1×25 mL), dried (MgSO₄), filtered, and concentrated under reduced pressure to provide the titled compound (2.51 g, 88%).

### Example 48B

### (S)-[1-methylcarbamoyl-2-(4-nitro-phenyl)-ethyl]-carbamic Acid Tert-butyl Ester

To a solution of (S)-N-boc-4-nitrophenylalanine (2.5 g, 8.1 mmol) and triethylamine (1.3 mL, 9.3 mmol) in THF (30 mL) at 0° C., was slowly added isobutylchloroformate (1.0 mL, 7.7 mmol) dropwise. After 10 min, methylamine (7 mL, 2M in THF) was added. The reaction was stirred for 0.5 hour, concentrated under reduced pressure. The residue was suspended in H₂O (25 mL) and filtered. The filter cake was washed with cold H₂O, and left on the filter to dry. The solid was suspended in ethyl acetate and concentrated under reduced pressure to provide the titled compound (1.74 g, 66%). ¹H NMR (300MHz, d₆-DMSO) δ8.16 (d, 1H, J=8.5), 7.89 (q, 1H, J=4.4), 7.52 (d, 2H, J=8.8), 7.01 (d, 1H, J=8.5), 4.16 (ddd, 1H, J=4.3, 9.6, 9.6), 3.08 (dd, 1H, J=4.6, 13.7), 2.85 (dd, 1H, J=10.3, 13.4), 2.59 (d, 3H, J=4.4), 1.27 (s, 9H); MS (ESI) m/z=324 (MH⁺).

### Example 48C

### (S)-2-amino-N-methyl-3-(4-nitro-phenyl)-propionamide

(S)-[1-methylcarbamoyl-2-(4-nitro-phenyl)-ethyl]-carbamic acid tert-butyl ester (400 mg, 1.24 mmol) was dissolved in trifluoroacetic acid. (2 mL) and stirred for 1 hour then concentrated under reduced pressure. The residue was taken up in ethyl acetate (30 mL) and H₂O (5 mL). Solid K₂CO₃was added to the mixture until an aliquot of ethyl acetate mixed with H₂O was basic. The organic phase was seperated, dried (MgSO₄), filtered, and concentrated under reduced pressure to provide the titled compound (900 mg). ¹H NMR (300 MHz, d₆-DMSO) δ8.14 (m, 2H), 7.88 (q, 1H, J=4.8), 7.48 (m, 2H), 3.43 (dd, 1, J=5.1, 8.1), 3.03 (dd, 1, J=5.1, 13.2), 2.78 (dd, 1, J =8.5, 13.2), 2.57 (d, 3, J=4.8), 2.40 (bs, 2); MS (ESI) m/z=224 (MH⁺).

### Example 48D

### 2-(S)-[2-(S)-acetylamino-3-(4-amino-3-ethyl-phenyl)-propionylamino]-N-methyl-3-(4-nitro-phenyl)-propionamide and 2-(S)-[2-(R)-acetylamino-3-(4-amino-3-ethyl-phenyl)-propionylamino]-N-methyl-3-(4-nitro-phenyl)-propionamide

To a solution of (±)2-acetylamino-3-(4-amino-3-ethyl-phenyl)-propionic acid (310 mg), (S)-2-amino-N-methyl-3-(4-nitro-phenyl)-propionamide (900 mg, 1.2 mmol) and triethylamine (400 μL) in N,N-dimethylformamide (3 mL) at 0° C. was added PyBOP (980 mg, 1.88 mmol). The mixture was stirred at ambient temperature for 2 hours, diluted with water (10 mL) and extracted with ethyl acetate (3×35 mL). The combined organic layers were dried (MgSO₄), filtered, concentrated under reduced pressure and crystallized from ethyl acetate to provide the titled compound (244 mg, 45%). 1H NMR (300 MHz, d₆-DMSO) 8:5 mixture of diastereomers, δ8.38 (d, 1H, J=8.8), 8.15-8.09 (m, 2H), 7.98-7.91 (m, 2H), 7.55 (q, 1H, J=4.7), 6.76-6.60 (m, 2H), 6.45 (t, 1H, J=8.1), 4.66 (bs, 1H), 4.50-4.45 (m, 1H), 4.26-4.36 (m, 1H), 3.18-3.07 (m, 1H), 3.04-2.99 (m, 3H). 2.99-2.85 (m, 2H), 2.73-2.67 (m, 1H), 2.60 (d, 3H, J=4.8), 2.56 (d, 3H, J=4.4), 2.41-2.32 (m, 3H), 1.75-1.71 (m, 6H), 1.09 (t, 3H, J=7.5), 1.08 (t, 3H, J=7.5); MS (ESI) m/z=454 (M−H), 478 (M+Na).

### Example 48 E

### 2-(4-{2-(R)-acetylamino-2-[(S)-1-methylcarbamoyl-2-(4-nitro-phenyl)-ethylcarbamoyl]-ethyl}-2-ethyl-phenylamino)-benzoic acid and 2-(4-{2-(S)-acetylamino-2-[(S)-1-methycarbamoyl-2-(4-nitro-phenyl)-ethylcarbamoyl]-ethyl}-2-ethyl-phenylamino)-benzoic Acid

To a mixture of 2-(S)-[2-(S)-acetylamino-3-(4-amino-3-ethyl-phenyl)-propionylamino]-N-methyl-3-(4-nitro-phenyl)-propionamide and 2-(S)-[2-(R)-acetylamino-3-(4-amino-3-ethyl-phenyl)-propionylamino]-N-methyl-3-(4-nitro-phenyl)-propionamide (244 mg, 0.536 mmol) in N,N-dimethylformamide (2 mL) was added diphenyliodonium-2-carboxylate monohydrate (219 mg (0.643 mmol), copper(II)acetate (14 mg, 14 mol %). The mixture was stirred at 100° C. under N₂ for 17 hours, poured into 0.2M NaOH (10 mL) and extracted with hexanes (3×5 mL), using methanol to break emulsions. The aqueous layer was acidified to a pH <3with 1M HCl and extracted with ethyl acetate (3×5 mL). The combined ethyl acetate layers were extracted with 1M HCl (1×3 mL), brine (1×3 mL), dried (MgSO₄), filtered, and concentrated under reduced pressure. The residue was precipitated from ethyl acetate to provide the titled compound as a mixture of both diastereomers (58 mg, 19%). ¹H NMR (300 MHz, d₆-DMSO) mixture of diastereomers, δ9.46 (s, 1), 8.49 (d, 1, J=8.8), 8.18 (d, 1, J=8.5), 8.12 (m, 2), 8.04-7.98 (m, 2), 7.87 (dd, 1, J=1.5, 8.0), 7.80-7.76 (m, 1), 7.55-7.53(m, 1), 7.49-7.39 (m, 2), 7.34-7.26 (m, 1), 7.18-7.02 (m, 4), 6.93(dd, 1, J=1.7, 8.1), 6.78 (t, 1, J=7.6), 4.54-4.43 (m, 3), 3.17-3.11 (m, 3), 3.04-2.96 (m, 2), 2.94-2.84 (m, 3), 2.73-2.63 (m, 2), 2.61 (d, 3, J=4.8), 2.57 (d, 3, J=4.4), 1.76-1.71 (m, 3), 1.11 (t, 3, J=7.5), 1.10 (t, 3, J=7.5); MS (ESI) m/z=576 (MH⁺).

### Example 48 F

### 2-[(4-{2-(R)-acetylamino-2-[(S)-1-methylcarbamoyl-2-(4-nitro-phenyl)-ethylcarbamoyl]-ethyl}-2-ethyl-phenyl)-oxalyl-amino]-benzoic acid and 2-[(4-{2-(S)-acetylamino-2-[(S)-1-methylcarbamoyl-2-(4-nitro-phenyl)-ethylcarbamoyl]-ethyl}-2-ethyl-phenyl)-oxalyl-amino]-benzoic Acid

To the mixture of 2-(4-{2-(R)-acetylamino-2-[(S)-1-methylcarbamoyl-2-(4-nitro-phenyl)-ethylcarbamoyl]-ethyl}-2-ethyl-phenylamino)-benzoic acid and 2-(4-{2-(S)-acetylamino-2-[(S)-1-methylcarbamoyl-2-(4-nitro-phenyl)-ethylcarbamoyl]-ethyl}-2-ethyl-phenylamino)-benzoic acid (33 mg, 0.057 mmol) in N,N-dimethylformamide (0.5 mL) was added N,N-diisopropylethylamine (30μL, 0.17 mmol) and ethyl oxalyl chloride (120 μL, 1.08 mmol). The mixture was stirred at ambient temperature for 2.5 hours followed by the addition of 2M NaOH (2 mL) and was stirred for 10 min. The mixture was diluted with water (3 mL), acidified to a pH <3 with 1M HCl and extracted with ethyl acetate (3×5 mL). The combined ethyl acetate layers were extracted with brine (1×5 mL), dried (MgSO₄), filtered, and concentrated under reduced pressure. The residue was precipitated with ethyl acetate to provide the titled compound (14 mg, 37%) ¹H NMR (300 MHz, d₆-DMSO) (Note: spectrum shows a complicated pattern of rotational isomers superimposed on a mixture of diastereomers) δ8.50-8.42 (ml), 8.30-8.09 (m, 4), 8.08-7.90 (m, 1), 7.89-7.76 (m, 1), 7.55-7.45 (m, 2), 7.45-7.25 (m, 1), 7.22-7.06 (m, 1), 7.04-6.95 (m, 1), 6.82-6.73(m, 1), 4.58-4.40 (m, 2), 3.20-3.05 (m, 4), 3.02-2.90 (m, 2), 2.72-2.56 (m, 3), 1.74-1.71 (m, 2), 1.25-1.18 (m, 1), 1.00-0.90 (m, 1); MS (ESI) m/z=648 (MH⁺), 670 (M+Na).

### EXAMPLE 49

### N-(3-carboxypropanoyl)-L-phenylalanyl-3-[(1E)-3-amino-3-oxo-1-propenyl]-4-[(carboxycarbonyl)(2-carboxyphenyl)amino]-N-pentyl-L-phenylalaninamide

### Example 49A

### 2-amino-3-(4-amino-3-iodo-phenyl)-N-pentyl-propionamide

The titled compound was prepared according to the procedure described in Example 43A, substituting [2-(4-amino-3-iodo-phenyl)-1-pentylcarbamoyl-ethyl]-carbamic acid tert-butyl ester from 38B for [2-(4-nitro-phenyl)-1-pentylcarbamoyl-ethyl]-carbamic acid tert-butyl ester.

### Example 49B

### {1-[2-(4-amino-3-iodo-phenyl)-1-pentylcarbamoyl-ethylcarbamoyl]-2-phenyl-ethyl}-carbamic Acid Tert-butyl Ester

The titled compound was prepared according to the procedure described for Example 35 D, substituting L-N-boc phenylalanine for 2-acetylamino-3-(4-amino-naphthalen-1-yl)-propionic acid from example 35C and substituting 2-amino-3-(4-amino-3-iodo-phenyl)-N-pentyl-propionamide from Example 49A for the amylamine.

### Example 49C

### N-{1-[2-(4-amino-3-iodo-phenyl)-1-pentylcarbamoyl-ethylcarbamoyl]-2-phenyl-ethyl}-succinamic Acid Methyl Ester

The titled compound was prepared according to the procedure described in Example 43A and 35D respectively, substituting {1-[2-(4-amino-3-iodo-phenyl)-1-pentylcarbamoyl-ethylcarbamoyl]-2-phenyl-ethyl}-carbamic acid tert-butyl ester for [2-(4-nitro-phenyl)-1-pentylcarbamoyl-ethyl]-carbamic acid tert-butyl ester in procedure 43A, and substituting the resulting amine for amylamine, and the methyl monosuccinate for 2-acetylamino-3-(4-amino-naphthalen-1-yl)-propionic acid in procedure 35D.

### Example 49D

### N-(3-carboxypropanoyl)-L-phenylalanyl-3-[( 1E)-3-amino-3-oxo-1-propenyl]-4-[(carboxycarbonyl)(2-carboxyphenyl)amino]-N-pentyl-L-phenylalaninamide

The titled compound was prepared according to the procedure described in Example 38 C-E, substituting the N-{1-[2-(4-Amino-3-iodo-phenyl)-1-pentylcarbamoyl-ethylcarbamoyl]-2-phenyl-ethyl}-succinamic acid methyl ester for the [2-(4-Amino-3-iodo-phenyl)-1-pentylcarbamoyl-ethyl]-carbamic acid tert-butyl ester in Example 38C. MS (ESI+) m/e 758 (M+H)⁺.

### EXAMPLE 50

### 3-(4-benzoylphenyl)-N-(tert-butoxycarbonyl)-L-alanyl-3-{4-[(carboxycarbonyl)(2-carboxyphenyl)amino]phenyl}-N˜1˜-pentyl-L-alaninamide

### Example 50A

### [2-(4-nitro-phenyl)-1-pentylcarbamoyl-ethyl]-carbamic Acid Tert-butyl Ester

A solution of N-(t-butoxycarbonyl)-4-nitro-L-phenylalanine (10.0 g, 32.2 mmol), amylamine (4.9 mL, 42 mmol), 1-[3-(dimethylamino)propyl]-3-ethylcarbodiimide hydrochloride (8.0 g, 42 mmol), and 3-hydroxy-1,2,3-benzotriazin-4(3H)-one (5.8 mg, 35 mmol) in N,N-dimethylformamide (20 mL) was adjusted to pH 7 with triethylamine and stirred overnight. The reaction was taken up in ethyl acetate, washed with H₂O (4×25 mL), 1 N HCl (1×25 mL) and aqueous sodium bicarbonate (1×25 mL). The organic layer was dried (MgSO₄), filtered and concentrated under reduced pressure. The residue was crystallized from ethyl acetate/hexanes to provide the titled compound. MS (ESI(+)) m/e 380 (M+H)⁺; ¹H NMR (300 MHz, DMSO-d₆) δ8.16 (d, 2H), 7.88 (t, 1H), 7.53(d, 2H), 6.99 (d, 1H), 4.22-4.14 (m, 1H), 3.09-2.97 (m, 3H), 2.92-2.83(m, 1H), 1.89-1.14 (m+s, 15H), 0.85 (t, 3H).

### Example 50B

### [2-(4-amino-phenyl)-1-pentylcarbamoyl-ethyl]-carbamic Acid Tert-butyl Ester

A solution of [2-(4-nitro-phenyl)-1-pentylcarbamoyl-ethyl]-carbamic acid tert-butyl ester (18.8 g, 49.5 mmol) and 10% Pd/C (0.5 g) in methanol (50 mL) under an atmosphere of hydrogen at 60 psi was shaken for 15 minutes. The mixture was filtered and the filtrate concentrated under reduced pressure to provide the titled compound. MS (ESI(+)) m/e 350 (M+H)⁺; ¹H NMR (300 MHz, DMSO-d₆) δ7.72 (t, 1H), 6.86 (d, 2H), 6.63 (d, 1H), 6.44 (d, 2H), 4.82 (s, 2H), 4.00-3.95 (m, 1H), 3.08-2.96 (m, 2H), 2.72 (dd, 1H), 2.58-2.53 (dd, 1H), 1.37-1.18 (m+s, 15 H), 0.85 (t, 3H).

### Example 50C

### 2-[4-(2-tert-butoxycarbonylamino-2-pentylcarbamoyl-ethyl)-phenylamino]-benzoic Acid

A mixture of [2-(4-amino-phenyl)-1-pentylcarbamoyl-ethyl]-carbamic acid tert-butyl ester (17.0 g, 48.6 mmol), diphenyliodonium-2-carboxylate monohydrate (20.0 g, 58.4 mmol), and copper(II) acetate (1.3 g, 7.3 mmol) in N,N-dimethylformamide (25 mL) was heated to 75° C. for 4 hours, cooled to room temperature and partitioned between aqueous 1N HCl and ethyl acetate (200 mL. 1:1). The organic layer was washed with H₂O (4 times), dried (MgSO₄), filtered, concentrated, and crystallized from ethyl acetate/hexanes to provide the titled compound. MS (ESI(+)) m/e 470 (M+H)⁺; ¹H NMR (300 MHz, DMSO-d₆) δ9.60 (bs, 1H), 7.88 (dd, 1H), 7.70 (t, 1H), 7.37-7.32 (m, 1H), 7.22 (d, 2H), 7.14 (bs+d, 3H), 6.86 (d, 1H), 6.78-6.72 (m, 1H), 4.13-4.07 (m, 1H), 3.11 -2.96 (m, 2H), 2.88 (dd, 1H), 2.72 (dd, 1H), 1.38-1.18 (m+s, 15H), 0.84 (t, 3H).

### Example 50D

### 2-[4-(2-allyloxycarbonylamino-2-pentylcarbamoyl-ethyl)-phenylamino]-benzoic Acid

A mixture of 2-[4-(2-tert-butoxycarbonylamino-2-pentylcarbamoyl-ethyl)-phenylamino]-benzoic acid (10.2 g, 21.7 mmol) and trifluoroacetic acid (50 mL) in methylene chloride (200 mL) was stirred for 3 hours, concentrated under reduced pressure. The residue (10.5 g, 21.7 mmol) and sodium bicarbonate (8.0 g, 95 mmol) were taken up in ethyl acetate/H₂O (250 mL, 3:2) and treated with allyl chloroformate (2.4 mL, 22.6 mmol). The mixture was stirred for 1 hour then diluted with ethyl acetate (100 mL). The organic layer was washed with H₂O (2×50 mL), dried (MgSO₄), filtered, concentrated under reduced pressure and crystallized from ethyl acetate/hexanes to provide the titled compound. MS (ESI(+)) m/e 454 (M+H)⁺; ¹H NMR (300 MHz, DMSO-d₆) δ13.01 (bs, 1H), 9.58 (bs, 1H), 7.92-7.90 (m, 2H), 7.38-7.34 (m, 2H), 2.27 (d, 2H), 7.19-7.16 (m, 3H), 6.76 (t, 1H), 5.88-5.81 (m, 1H), 5.23(d, 1H), 5.14 (d, 1H), 4.42 (d, 2H), 4.22-4.17 (m, 1H), 3.12-2.97 (m, 2H), 2.94 (dd, 1H), 2.68 (dd, 1H), 1.40-1.34 (m, 2H), 1.29-1.17 (m, 4H), 0.85 (t, 3H).

### Example 50E

### 2-{[4-(2-allyloxycarbonylamino-2-pentylcarbamoyl-ethyl)-phenyl]-tert-butoxyoxalylamino}-benzoic Acid

To a solution of 2-[4-(2-allyloxycarbonylamino-2-pentylcarbamoyl-ethyl)-phenylamino]-benzoic acid (7.0 g, 14.5 mmol), diisopropylethylamine (8.8 mL, 50.7 mmol) ) in dichloromethane (25 mL) was added t-butyloxalyl chloride (5.2 g, 31.9 mmol) and stirred overnight. The reaction was partitioned between ethyl acetate (75 mL) and aqueous 1N HCl( 50 mL). The organic layer was washed with H₂O (2×30 mL), dried (MgSO₄), filtered and concentrated. The crude material was precipitated from dichloromethane to provide the titled compound as a 1:1 mixture of rotamers. MS (ESI(−)) m/e 580 (M−H)⁺; ¹H NMR (300 MHz, DMSO-d₆) δ13.17 (bs, 1H), 8.05-8.02 and 7.87-7.83(2m, 1H total), 7.96-7.89 (m, 1H), 7.72-7.34 (m, 6H), 7.28-7.18 (m, 2H), 5.88-5.75 (m, 1H), 5.24-5.08 (m, 2H), 4.43-4.28 (m, 2H), 4.17-4.08 (m, 1H), 3.07-2.99 (m, 2H), 2.96-2.87 (m, 1H), 2.78-2.69 (m, 1H), 1.43-1.32 (m, 2 H), 1.30-1.18 (m, 4H), 1.13 and 1.11 (2s, 9H total), 0.85 (t, 3H).

### Example 50F

### 2-{[4-(2-allyloxycarbonylamino-2-pentylcarbamoyl-ethyl)-phenyl]-tert-butoxyoxalyl-amino}-benzoic Acid Benzhydryl Ester

To a solution of 2-{[4-(2-allyloxycarbonylamino-2-pentylcarbamoyl-ethyl)-phenyl]-tert-butoxyoxalylamino}-benzoic acid (6.46 g, 11.1 mmol) in acetone (25 mL) was added diphenylmethyldiazomethane (3.25 g, 16.6 mmol) and stirred overnight. The solution was concentrated under reduced pressure and purified on silica using 0-10% ethyl acetate/dichloromethane as eluent to provide the titled compound as a 1:1 mixture of rotamers. MS (ESI(+)) m/e 765 M+H₂O, 748 (M+H)⁺; ¹H NMR (300 MHz, DMSO-d₆) δ8.27-8.24 and 8.08-8.05 (2m, 1H total), 7.95-7.88 (m, 1H), 7.81-7.27 (m, 16H), 7.18-7.14 (m, 1H), 7.07-7.03 (m, 1H), 6.99 and 6.44 (2s, 1H total), 5.87-5.74 (m, 1H), 5.23-5.15 (m, 1H), 5.11-5.07 (m, 1H), 4.43-4.28 (m, 2H), 4.15-4.08 (m, 1H), 3.07-2.98 (m, 1H), 2.93-2.85 (m, 1H), 2.76-2.67 (m, 1H), 1.41-1.29 (m, 2H), 1.27-1.15 (m, 4H), 1.11 and 0.99 (2s, 9H total), 0.84 (t, 3H).

### Example 50G

### 2-{[4-(2-amino-2-pentylcarbamoyl-ethyl)-phenyl]-tert-butoxyoxalyl-amino}-benzoic Acid Benzhydryl Ester

To a solution of 2-{[4-(2-allyloxycarbonylamino-2-pentylcarbamoyl-ethyl)-phenyl]-tert-butoxyoxalyl-amino}-benzoic acid benzhydryl ester (2.50 g, 3.35 mmol) in methylene chloride (50 mL) was added tetrakis(triphenylphosphine)palladium(O) (193 mg, 0.167 mmol) followed by piperidine (0.829 mL, 8.37 mmol) and then stirred for 1 hour. The mixture was concentrated under reduced pressure and purified on silica using 1-3% 2-propanol/dichloromethane to provide the titled compound as a 1:1 mixture of rotamers. MS (ESI(+)) m/e 664 (M+H)⁺; ¹H NMR (300 MHz, DMSO-d₆) δ8.28-8.25 and 8.09-8.06 (2m, 1H total), 7.82-6.94 (m, 21H), 3.93-3.73 (m, 1H), 3.03-2.97 (m, 2H), 2.90-2.82 (m, 1H), 2.63-2.55 (m, 1H), 1.35-1.28 (m, 2H), 1.23-1.14 (m, 4H), 1.16 and 0.99 (2s, 9H total), 0.85-0.78 (m, 3H).

### Example 50H

### 2-[(4-{2-[2-(3-benzoyl-phenyl)-2-tert-butoxycarbonylamino-acetylamino]-2-pentylcarbamoyl-ethyl}-phenyl)-tert-butoxyoxalyl-amino]-benzoic Acid Benzhydryl Ester

A mixture of N-(t-butoxycarbonyl)-4-benzoyl-L-phenylalanine and 2-{[4-(2-amino-2-pentylcarbamoyl-ethyl)-phenyl]-tert-butoxyoxalyl-amino}-benzoic acid benzhydryl ester were processed as described in Example 50A to provided the titled compound obtained as a 1:1 mixture of rotamers. MS (ESI(−)) m/e 1014 (M−H)⁺; ¹H NMR (300 MHz, DMSO-d₆) δ8.27-7.88 (m, 3H), 7.72-6.83(m, 28 H), 4.52-4.43(m, 1H), 4.23-4.16 (m, 1H), 3.12-2.68 (m, 6H), 1.33-1.07 (m, 6H), 1.26 (s, 9H), 1.14 and 0.98 (2s, 9H total), 0.83-0.77 (m, 3H).

### Example 50I

### 3-(4-benzoylphenyl)-N-(tert-butoxycarbonyl)-L-alanyl-3-{4-[(carboxycarbonyl)(2-carboxyphenyl)amino]phenyl}-N˜1˜-pentyl-L-alaninamide

A solution of 2-[(4-{2-[2-(3-benzoyl-phenyl)-2-tert-butoxycarbonylamino-acetylamino]-2-pentylcarbamoyl-ethyl}-phenyl)-tert-butoxyoxalyl-amino]-benzoic acid benzhydryl ester (515 mg, 0.507 mmol) and trifluoroacetic acid (3 mL) in dichloromethane (3 mL) was stirred for 3 hours, concentrated to constant weight under reduced pressure. A solution of the residue (351 mg, 0.435 mmol), triethylamine (182 μL, 1.31 mmol) and di-t-butyl dicarbonate (101 μL, 0.439 mmol) in methylene chloride (3 mL) was stirred overnight, concentrated under reduced pressure and purified by reverse phase HPLC eluting with 0-70% acetonitrile/(10 mM ammonium acetate in H₂O) to provide the titled compound. MS (ESI(−)) m/e 791 (M−H)⁺; ¹H NMR (300 MHz, DMSO-d₆) δ2.98 (d, 1H), 2.89 (t, 1H), 7.73-7.53(m, 8H), 7.46-7.35 (m, 7H), 7.16-7.03(m, 5H), 4.48-4.40 (m, 1H), 4.23-4.15 (m, 1H), 3.01-2.71 (m, 6H), 1.29 (s, 9H), 1.32-1.08 (m, 6H), 0.82 (t, 3H).

### EXAMPLE 51

### N-acetyl-4-[(carboxycarbonyl)(2-carboxyphenyl)amino]-3-(2-hydroxyethyl)-N-[4-(methylsulfonyl)benzyl]phenylalaninamide

The titled compound was prepared according to the procedure described in Example 35 C-E, substituting the 4-methanesulfonyl-benzylamine for the amylamine, and 2-acetylamino-3-[4-amino-3-(2-hydroxy-ethyl)-phenyl]-propionic acid for 2-acetylamino-3-(4-amino-naphthalen-1-yl)-propionic acid used in Example 35D. MS (ESI+) m/e 648 (M+Na)⁻; 626 (M+H)⁻; ¹H NMR (300 MHz, DMSO-d₆) (A mixture of rotamers) δ8.63 (brm, 1H), 8.28-8.13 (m, 2H), 8.00-7.77 (m, 3H), 7.60-6.74 (m, 7H), 5.25-4.20 (m, 5H), 3.18 (s, 3H), 3.14-2.62 (m, 4H), 1.88-1.73(m, 3H).

### EXAMPLE 52

### 2-[[7-(aminocarbonyl)-1-naphthyl](carboxycarbonyl)amino]benzoic Acid

### Example 52A

### 8-nitro-naphthalene-2-carboxylic Acid Ethyl Ester

A cold nitric acid solution [prepared by adding fuming nitric acid (5.15 mL, 12.0 mmol) dropwise over 15 minutes to cooled (−10° C.) Ac₂O (25 mL)] was added dropwise over 10 minutes to a cooled (−10° C.) solution of 2-naphthoic acid (19.0 g, 11.0 mmol) in concentrated H₂SO₄ (5 mL) and acetic anhydride (200 mL). The mixture was stirred at ambient temperature for 5 hours, poured into ice water (1 L) and filtered. The precipitate was washed with H₂O and methanol and dried under vacuum at 50° C. for 16 hours. The residue was taken up in ethanol (250 mL) and concentrated H₂SO₄ (2 mL) and the mixture was refluxed for 3 days. The mixture was cooled, filtered, reduce in volume and filtered. The filter cakes were combined and crystallized from ethyl acetate to provide the titled compound. MS (ESI(−)) m/e 245 (M−H)⁺; ¹H NMR (300 MHz, DMSO-d₆) δ9.05 (s, 1H), 8.48-8.43 (m, 2H), 8.30 (d, 1H), 8.16 (dd, 1H), 7.86 (t, 1H), 4.42 (q, 2H), 1.39 (t, 3H).

### Example 52B

### 8-nitro-naphthalene-2-carboxylic Acid

A solution of 8-nitro-naphthalene-2-carboxylic acid ethyl ester (0.95 g, 3.9 mmol) in 4:1 methanol:THF (60 mL) was treated with 2 M NaOH (10 mL) and stirred for 16 hours. The mixture was concentrated under reduced pressure and partitioned between ethyl acetate and aqueous 2 N HCl. The organic layer was washed with H₂O (2×30 mL), dried (MgSO₄), filtered and concentrated under reduced pressure to give the titled compound. MS (ESI(−)) m/e 216 (M−H)⁺; ¹H NMR (300 MHz, DMSO-d₆) δ13.5 (bs, 1H), 9.05 (s, 1H), 8.48-8.42 (m, 2H), 8.29 (d, 1H), 8.17 (dd, 1H), 7.85 (t, 1H).

### Example 52C

### 8-nitro-naphthalene-2-carboxylic Acid Amide

A mixture of 8-nitro-naphthalene-2-carboxylic acid (0.82 g, 3.8 mmol), pyridine (1 drop), N,N-dimethylformamide (3drops) in methylene chloride (3 mL) was treated with thionyl chloride (303 μL, 4.2 mmol) and stirred 2 days. The mixture was dried (Na₂SO₄), filtered and concentrated under reduced pressure. To a solution of the residue (650 mg, 2.76 mmol) in THF (150 mL) was added concentrated NH₄OH (0.5 mL) and stirred 30 minutes. The reaction was concentrated under reduced pressure and the residue purified on silica eluting with 5% methanol/dichloromethane to give the titled compound. MS (ESI(+)) m/e 217 (M+H)⁺; ¹H NMR (300 MHz, DMSO-d₆) δ8.87 (m, 1H), 8.42 (d, 1H), 8.37 (dd, 1H), 8.30 (bs, 1H), 8.25 (d, 1H), 8.13 (dd, 1H), 7.80 (t, 1H), 7.65 (bs, 1H).

### Example 52D

### 8-amino-naphthalene-2-carboxylic Acid Amide

A solution of 8-nitro-naphthalene-2-carboxylic acid amide (330 mg, 1.53 mmol) and 5% Pd/C (30 mg) in methanol (3 mL) was stirred under an atmosphere of hydrogen. The catalyst was filtered and the filtrate was concentrated under reduced pressure to provide the titled compound. MS (ESI(+)) m/e 187 (M+H)⁺; ¹H NMR (300 MHz, DMSO-d₆) δ8.66 (s, 1H), 7.88 (bs, 1H), 7.85 (dd, 1H), 7.75 (d, 1H), 7.35 (bs, 1H), 7.38 (t, 1H), 7.10 (d, 1H) 6.72 (d, 1H), 5.82 (bs, 2H).

### Example 52E

### 2-(7-carbamoyl-naphthalen-1-ylamino)-benzoic Acid

The titled compound was prepared according to the procedure described in Example 50 C, substituting 8-amino-naphthalene-2-carboxylic acid amide for [2-(4-amino-phenyl)-1-pentylcarbamoyl-ethyl]-carbamic acid tert-butyl ester. MS (ESI(+)) m/e 307 (M+H)⁺; ¹H NMR (300 MHz, DMSO-d₆) δ10.07 (s, 1H), 8.62 (s, 1H), 8.16 (bs, 1H), 8.05-7.95 (m, 3H), 7.79 (dd, 1H), 7.64-7.57 (m, 2H), 7.45 (bs, 1H), 7.38-7.33 (m, 1H), 6.99 (d, 1H), 6.83-6.78 (m, 1H).

### Example 52F

### 2-[(7-carbamoyl-naphthalen-1-yl)-ethoxyoxalyl-amino]-benzoic Acid

The titled compound was prepared according to the method described by Example 50 E substituting 2-(7-carbamoyl-naphthalen-I-ylamino)-benzoic acid for 2-[4-(2-allyloxycarbonylamino-2-pentylcarbamoyl-ethyl)-phenylamino]-benzoic acid, and substituting ethyl oxalylchloride for t-butyloxalyl chloride. The material was purified by reverse phase HPLC with 0% to 70% acetonitrile/(0.1% trifluoroacetic acid in H₂O). The compound was a 3:2 mixture of rotamers. MS (ESI(+)) m/e 407 (M+H)⁺; ¹H NMR (300 MHz, DMSO-d₆) δ8.94 and 8.58 (2s, 1H total), 8.13-7.38 (m, 10H), 7.26 and 7.04 (2d, 1H total), 4.06 and 3.74 (2q, 2H total), 1.00 and 0.51 (2t, 3H total).

### Example 52G

### 2-[[7-(aminocarbonyl)-1-naphthyl](carboxycarbonyl)amino]benzoic Acid

A solution of 2-[(7-carbamoyl-naphthalen-1-yl)-ethoxyoxalyl-amino]-benzoic acid (30 mg, 0.074 mmol) and aqueous 2 M NaOH (0.5 mL) in methanol (2 mL) was stirred for 3 hours, acidified with aqueous 2N HCl and purified by reverse phase HPLC eluting with 0-70% acetonitrile/(0.1% trifluoroacetic acid in H₂O) to provide the titled compound as a 3:2 mixture of rotamers. MS (ESI(+)) m/e 396 M+H₂O, 379 (M+H)⁺; ¹H NMR (300 MHz, DMSO-d₆) δ8.97 and 8.58 (2s, 1H total), 8.16-7.99 (m, 4H), 7.89-7.24 (m, 7H), 7.26 and 6.98 (2d, 1H total).

### EXAMPLE 53

### N-acetyl-4-[(carboxycarbonyl)(2-carboxyphenyl)amino]-3-isopropyl-N-[4-(methylsulfonyl)benzyl]phenylalaninamide and 4-[(carboxycarbonyl)(2-carboxyphenyl)amino]-1-acetyl-6-(3-isopropylbenzyl)-4-[4-(methylsulfonyl)benzyl]-2,3,5-piperazinetrione

The titled mixture of compounds was prepared according to the method described in Example 35 D by substituting 4-methanesulfonyl-benzylamine HCl salt for the amylamine. MS (ESI(+)) m/e 624 (M+H)⁺ and 678 (M+H)⁺; ¹H NMR (500 MHz, DMSO-d₆) δ6.9-8.0 (m, 7H), 3.3-4.1 (br m, 4H), 3.18 (s, 31H), 1.771.80 (two s, 3H), 1.10-1.30 (m, 6H), 0.61 (m, 2H).

### EXAMPLE 54

### 2-[(carboxycarbonyl)(7-hydroxy-1-naphthyl)amino]-4-hydroxybenzoic Acid

### Example 54 A

### 2-bromo-4-nitrobenzoic Acid

To a solution of 2-bromo-4-nitrotoluene (7.0 g, 32 mmol) that was dissolved in if sulfuric acid (98%, 50 mL) and placed in a water bath to maintain ambient temperature was added dropwise a solution of chromium trioxide (7.5 g, 75 mmol) in water (8 mL). Following addition, the mixture was poured onto 200 mL of ice, the precipitate was collected and washed with water (1×100 mL). The crude product was taken up in diethyl ether (50 mL), extracted with aqueous NaHCO₃solution (2×25 mL). The combined bicarbonate layers were acidified by the addition of 12M HCl, extracted with diethyl ether (2×25 mL). The second set of diethyl ether layers was washed with brine (1×25 mL), dried (MgSO₄), filtered, and concentrated under reduced pressure to provide the titled compound 4.0 g (51%). ¹H NMR (300 MHz, d₆-DMSO) δ8.48 (d, 1H, J=2.4), 8.28 (dd, 1H, J=2.0, 8.5), 7.95 (d, 1H, J=8.5); MS (ESI) m/z=244, 246 (M−H).

### Example 54B

### methyl 2-bromo-4-nitrobenzoate

To a solution containing 2-bromo-4-nitrobenzoic acid (1.0 g, 4.06 mmol) and K₂CO₃ (560 mg) in N,N-dimethylformamide (5 mL) was added methyl iodide (500 μL, 8.03 mmol). The mixture was stirred at ambient temperature for 1 hour, poured into water (30 mL) and extracted with diethyl ether (3×10 mL). The combined ether layers were washed with water (1×10 mL), brine (1×10 mL), dried (MgSO₄), filtered, and concentrated under reduced pressure to provide the titled compound (970 mg, 92%). ¹H NMR (300 MHz, CDCl₃) δ8.52 (d, 1H, J=2.4), 8.21 (dd, 1, J=2.0, 8.5), 7.92 (d, 1, J=8.8), 3.99 (s, 3); MS (ESI) m/z=259 (M−H).

### Example 54C

### methyl-2-bromo-4-aminobenzoate

A solution of methyl 2-bromo-4-nitrobenzoate (970 mg, 3.73 mmol), iron powder 1.25 g (22.4 mmol) and ammonium chloride (239 mg, 4.48 mmol) in aqueous 2-propanol (20%, 15 mL) was heated to reflux for 30 minutes, cooled, filtered, and concentrated under reduced pressure. The residue was partitioned between diethyl ether (20 mL) and water (5 mL). The organic layer was washed with brine (1×5 mL), dried (MgSO₄), filtered, and concentrated to provide the titled compound (813 mg, 95%).

### Example 54D

### methyl-2-bromo-4-hydroxybenzoate

To a mixture of methyl-2-bromo-4-aminobenzoate (813 mg, 3.53 mmol) in water (10 mL) and 98% H₂SO₄ (1 mL) at 0° C. was added a solution of aqueous NaNO₂ (244 mg, 3.53 mmol) dissolved in a minimum amount of water, via pipette below the surface of the reaction. After 15 minutes, the mixture was filtered, and the filter cake washed with H₂O (10 mL). The combined filtrate was heated to reflux for 15 minutes, stirred at ambient temperature for 18 hours and extracted with diethyl ether (3×10 mL). The combined organic layers were washed with brine (1×5 mL), dried (MgSO₄), filtered, and concentrated under reduced pressure to provide the titled compound (444 mg, 54%).

Example 54E 2-bromo-4-hydroxybenzoic acid To methyl 2-bromo-4-hydroxybenzoate (212 mg, 0.918 mmol) was added 30% HBr (w/w) in acetic acid (3 mL). The solution was heated to 100° C. for 5 hours, poured into H₂O (10 mL) and extracted with diethyl ether (3×5 mL). The combined ether layers were washed with water (1×5 mL), brine (1×5 mL), dried (MgSO₄), filtered, and concentrated under reduced pressure to provide the titled compound (162 mg, 81%). ¹H NMR (500 MHz, CDCl₃) δ7.86 (d, 1H, J=8.7), 7.14 (d, 1H, J=2.5), 6.79 (d, 1H, J=2.5, 8.7); MS (ESI) m/z=215, 217 (M−H).

### Example 54F

### benzyl-2-bromo-4-benzyloxybenzoate

To 2-bromo-4-hydroxybenzoic acid (1 62 mg, 0.747 mmol) was added 1M KOH in methanol (1.5 mL). The mixture was stirred until all material had dissolved, then concentrated under reduced pressure. The residue was taken up in N,N-dimethylformamide (3 mL) and methanol (0.5 mL) followed by the addition of benzyl bromide (300 μL, 1.68 mmol) and K₂CO₃ (150 mg, 1.08 mmol). The mixture was heated to 90° C. for 10 min, poured into H₂O (10 mL) and extracted with diethyl ether (3×5 mL). The combined organic layers were washed with water (1×5 mL), brine (1×5 mL), dried (MgSO₄), filtered, and concentrated under reduced pressure to provide the titled compound (285 mg, 96%).

### Example 54G

### 2-(tert-butyldimethylsilyloxy)-8-aminonaphthalene

A mixture of 8-amino-2-naphthol (3.18 g, 20.0 mmol), tert-butyl-dimethylsilyl chloride (3.6 g, 24 mmol) and imidazole (2.8 g, 41 mmol) in N,N-dimethylformamide(20 mL) was stirred at ambient temperature for 30 minutes, poured into water (70 mL) and aqueous 1M HCl (30 mL) and extracted with diethyl ether (3×30 mL). The combined ether layers were washed with water (1×30 mL), dried (MgSO₄), filtered, and concentrated under reduced pressure. The product was purified on silica using 15% ethyl acetate/hexanes as eluent to provide the titled compound (4.39 g, 80%). ¹H NMR (300 MHz, CDCl₃) δ7.68 (d, 1H, J=8.8),7.27 (d, 1H, J=8.8), 7.17 (d, 1H, J=2.4), 7.15 (dd, 1H, J=7.1, 8.1), 7.05 (dd, 1H, J=2.4, 8.8), 6.76 (dd,1H, J=1.0, 7.1), 4.11 (bs, 2H), 1.03(s, 9H), 0.25 (s, 6H); MS (ESI) m/z=274 (MH⁺).

### Example 54H

### 4-benzyloxy-2-[7-(tert-butyl-dimethyl-silanyloxy)-naphthalen-1-ylamino]-benzoic Acid Benzyl Ester

A mixture of benzyl-2-bromo-4-benzyloxybenzoate (285 mg, 0.717 mmol), 2-(tertbutyldimethylsilyloxy)-8-aminonaphthalene (196 mg, 0.717 mmol), tris-(dibenzylideneacetone)dipalladium (4 mg, 0.004 mmol), (2′-dicyclohexylphosphanyl-biphenyl-2-yl)-dimethyl-amine (6 mg, 0.015 mmol) and 60% NaH in mineral oil (45 mg, 1.1 mmol) in toluene (3 mL) was heated to reflux under N₂ for 1 hour, poured into water (10 mL) acidified to a pH <3with aqueous 1M HCl and extracted with diethyl ether (3×5 mL). The combined ether layers were washed with brine (1×5 mL), dried (MgSO₄), filtered, concentrated under reduced pressure and purified silica gel eluting with 10% ethyl acetate/hexanes to provide the titled compound.

### Example 54I

### 4-hydroxy-2-(7-hydroxy-naphthalen-1-ylamino)-benzoic Acid

A mixture of 4-benzyloxy-2-[7-(tert-butyl-dimethyl-silanyloxy)-naphthalen-1-ylamino]-benzoic acid benzyl ester and tetrabutylammonium fluoride (90 mg, 1.0 mmol) in THF (2 mL) was stirred for 5 minutes, then concentrated under reduced pressure. The residue was taken up in aqueous 0.2M HCl (10 mL) and extracted with diethyl ether (2×5 mL). The combined ether layers were dried (MgSO₄), filtered, concentrated under reduced pressure and purified on silica gel eluting with 20% ethyl acetate/hexanes. A mixture of the residue, 10% Pd/C (10 mg), 60% HClO₄ (3drops) in 2-propanol (2 mL) was stirred under 1 atmosphere of H₂ for 18 hours, filtered and concentrated under reduced pressure. The product was taken up in toluene and concentrated under reduced pressure to remove water to provide the titled compound (134 mg, 45%). HPLC/MS analysis showed a peak corresponding to the correct mass, and a minor contaminant peak.

### Example 54J

### 4-hydroxy-2-[(7-hydroxy-naphthalen-1-yl)-oxalyl-amino]-benzoic Acid

To 4-hydroxy-2-(7-hydroxy-naphthalen-1-ylamino)-benzoic acid (134 mg, 0.454 mmol) in N,N-dimethylformamide (3 mL) was added triethylamine (0.6 mL, 4.3 mmol) followed by ethyl oxalyl chloride (0.3 mL, 2.7 mmol). The reaction was stirred at ambient temperature for 25 minutes, 2M NaOH (5 mL) was added, stirred for an additional 30 minutes, then diluted with water (15 mL) and extracted with diethyl ether (2×5 mL). The aqueous layer was acidified to a pH <3with 1M HCl, extracted with di ethyl ether (3×5 mL). The aqueous layer was extracted with ethyl acetate (3×5 mL). The combined ethyl acetate layers were washed with brine (1×5 mL), dried (MgSO₄), filtered, and concentrated under reduced pressure to provide the titled compound (37 mg, 22%). ¹H NMR (300 MHz, d₆-DMSO) mixture of rotamers δ10.30 (s, 1H), δ10.30 (s, 1H), 10.10 (s, 1H), 10.06 (s, 1H), 9.94 (s,1H), 7.95-7.76 (m, 6H), 7.56 (d, I1H, J=7.5), 7.50 (d, 1H, J=1.4), 7.41-7.23 (m, 5H), 7.19-7.11 (m, 3H), 6.76 (dd, 1H, J=2.5, 8.7), 6.70 (dd, J=2.4, 8.5), 6.61 (d, 1, J=2.4), 6.33(bs, 1); MS (ESI) m/z=368 (MH⁻), 385 (M+NH4⁺).

### EXAMPLE 55

### N-acetyl-4-[1(carboxycarbonyl)(2-carboxyphenyl)amino]-3-ethyl-N-{5-oxo-5-[(1-phenylethyl)amino]pentyl}phenylalaninamide

### Example 55A

### 2-Acetylamino-acrylic Acid Benzyl Ester

To a mixture of 2-acetamidoacrylic acid (10.3 g, 80.0 mmol) and K₂CO₃(10 g, 72.5 mmol) in N,N-dimethylformamide (50 mL) was added benzyl bromide (8.7 ml, 72.5 mmol) at room temperature then stirred at room temperature for 3 hours. The mixture was partitioned between ethyl acetate and water (50 mL, 1:1), the aqueous layer was extracted with ethyl acetate (2×45 mL). The combined organic layers was washed with brine (2×25 mL), dried (MgSO₄), filtered and concentrated under reduced pressure to provide titled compound. MS (ESI(+)) m/e 220(M+H)⁺; ¹H NMR (300 MHz, DMSO-d₆) δ9.37 (s, 1H), 7.43-7.30 (m, 5H), 6.13 (s, 1H), 5.70 (s, 1H), 5.23(s, 2H), 2.01 (s, 3H).

### Example 55B

### 2-acetylamino-3-(4-amino-3-ethyl-phenyl)-acrylic Acid Benzyl Ester

To 2-acetylamino-acrylic acid benzyl ester (80.0 mmol) in acetonitrile (200 mL) was added Pd(OAc)₂ (488 mg, 2.18 mmol), (o-Tol)₃P (1.32 g, 4.35 mmol), Et₃N (20 mL) followed by addition of 4-bromo-2-ethylaniline (14.5 g, 72.5 mmol). The reaction mixture was heated to reflux overnight, concentrated under reduce pressure, taken up in ethyl acetate, washed with aqueous NaHCO₃, dried (MgSO₄), filtered and concentrated under reduced pressure. The residue was precipitated from ethyl acetate/hexane to provide the titled compound (6.3 g). The filtrate was precipitated a second time to provide and additional 5 g of the titled compound. MS (ESI(+)) m/e 339 (M+H)⁺, ¹H NMR (300 MHz, DMSO-d₆) δ9.31 (s, 1H), 7.40-7.20 (m, 8H), 6.59 (d, 1H), 5.52 (s, 2H), 5.16 (s, 2H), 2.42 (q, 2H), 1.98 (s, 3H), 1.13 (t, 3H).

### Example 55C

### 2-acetylamino-3-(4-amino-3-ethyl-phenyl)-propionic Acid

A mixture of 2-acetylamino-3-(4-amino-3-ethyl-phenyl)-acrylic acid benzyl ester (5 g) and 10% Pd-C (100 mg) in methanol (50 mL) was stirred under an atmosphere of hydrogen (4 atmospheres) at ambient temperature overnight to provide the titled compound. MS (ESI(+)) m/e 251 (M+H)⁺; ¹H NMR (300 MHz, DMSO-d₆) δ8.02 (d, 1H), 6.77-6.70 (m, 2H), 6.50 (d, 1H), 4.31-4.21 (m, 1H), 2.84 (dd, 1H), 2.65 (dd, 1H), 2.39 (q, 2H), 1.78 (s, 3H), 1.10 (t, 3H).

### Example 55D

### 2-acetylamino-3-(4-amino-3-ethyl-phenyl)-propionic Acid Allyl Ester

A mixture of 2-acetylamino-3-(4-amino-3-ethyl-phenyl)-propionic acid (2.0 g, 8.0 mmol), Cs₂CO₃(2.61 g, 8.0 mmol) and allyl bromide (692 ul, 8.0 mmol) in N,N-dimethylformamide (40 mL) was stirred at room temperature for 3 hours, concentrated under reduce pressure and partitioned between ethyl acetate and water (100 mL, 1:1). The organic phase was washed with brine (1×50 mL), dried (MgSO₄), filtered and concentrated under reduced pressure. The residue was purified by on silica gel with ethyl acetate/hexane (5:3) to provide titled compound (1.44 g). MS (ESI(+)) m/e 291 (M+H)⁺; ¹H NMR (300 MHz, DMSO-d₆) δ8.23 (d, 1H), 6.77-6.70 (m, 2H), 6.50 (d, 1H), 5.90-5.76 (m, 1H), 5.30-5.15 (m, 2H), 4.67 (s, 2H), 4.54-4.50 (m, 2H), 4.38-4.30 (m, 1H), 2.77 (dddd, 2H), 2.39 (q, 2H), 1.80 (s, 3H), 1.10 (t, 3H).

### Example 55E

### 2-{[4-(2-acetylamino-2-allyloxycarbonyl-ethyl)-2-ethyl-phenyl]-tert-butoxyoxalyl-amino}-benzoic Acid

The titled compound was prepared according to the method described in Example 3by substituting 2-acetylamino-3-(4-amino-3-ethyl-phenyl)-propionic acid allyl ester for 4-methoxyaniline and t-butyl oxalyl chloride for ethyl oxalyl chloride. MS (APCI (+)) m/e 539 (M+H)⁺.

### Example 55F

### 2-{[4-(2-acetylamino-2-allyloxycarbonyl-ethyl)-2-ethyl-phenyl]-tert-butoxyoxalyl-amino}-benzoic Acid Benzhydryl Ester

To 2-{[4-(2-acetylamino-2-allyloxycarbonyl-ethyl)-2-ethyl-phenyl]-tert-butoxyoxalyl-amino}-benzoic acid in acetone was added diphenyldiazomethane (until all starting material was consumed as evident by monitoring via TLC). The reaction mixture was concentrated under reduced pressure, purified on silica gel using ethyl acetate as eluent to provide the titled compound. MS (ESI(+)) m/e 705 (M+H)⁺; ¹H NMR (300 MHz, DMSO-d₆) δ8.51-8.01 (m, 2H), 7.73-6.86 (m, 16H), 5.93-5.78 (m, 1H), 5.34-5.10 (m, 2H), 4.57-4.40 (m, 3H), 3.10-2.84 (m, 2H), 2.58-2.42 (m, 2H), 1.82-1.77 (m, 3H), 1.22-0.78 (m, 3H), 1.07, 1.05, 1.00 (s, s, s, 9H).

### Example 55G

### 2-{[4-(2-acetylamino-2-carboxy-ethyl)-2-ethyl-phenyl]-tert-butoxyoxalyl-amino}-benzoic Acid Benzhydryl Ester

A mixture of 2-{[4-(2-acetylamino-2-allyloxycarbonyl-ethyl)-2-ethyl-phenyl]-tert-butoxyoxalyl-amino}-benzoic acid benzhydryl ester (3.4 g, 4.8 mmol), Pd(Ph₃P)₄ (166 mg, 0.144 mmol) and morpholine (0.5 ml, 5.8 mmol) in dichloromethane (25 mL) was stirred under N₂ atmosphere for 2 hours, partitioned between ethyl acetate and water (75 mL, 1:1). The organic phase was washed with 1N HCl (1×25 mL), brine (1×25 mL), dried (MgSO₄), filtered and concentrated under reduced pressure to provide the titled compound (3.3 g). MS (ESI(+)) m/e 665 (M+H)⁺; ¹H NMR (300 MHz, DMSO-d₆) δ12.67 (s, 1H), 8.51-7.98(m, 2H), 7.73-6.86 (m, 16H), 4.53-4.33 (m, 1H), 3.12-2.76 (m, 2H), 2.58-2.42 (m, 2H), 1.82-1.77 (m, 3H), 1.22-0.78 (m, 3H), 1.06, 1.04, 1.00 (s, s, s, 9H).

### Example 55H

### 5-tert-butoxycarbonylamino-pentanoic acid 2-trimethylsilanyl-ethyl Ester

A mixture of boc-d-aminovaleric acid (13.0 g, 59.5 mmol), pyridine (45 mL), (2-trimethylsilyl)ethanol (10.3 ml, 71.8 mmol) and dicyclohexylcarbodiimide (13.5 g, 65.4 mmol) in acetotnitrile (60 mL) was stirred cold (ice bath) for 1 hour and then kept in a refrigerator overnight. The suspension was filtered and the filtrate concentrated under reduced pressure to remove most of pyridine, diluted with ethyl acetate and washed with 1N HCl, saturated NaHCO₃. The organic phase was dried (MgSO₄), filtered and concentrated. The concentrate was purified by flash column chromatography on silica gel with hexane/ethyl acetate (4:1) to provide the desired product (15.3 g). MS (ESI(+)) m/e 318 (M+H)⁺; ¹H NMR(300 MHz, DMSO-d₆) δ6.77 (t, 1H), 4.11-4.03(m, 2H), 3.30 (m, 2H), 2.91-2.83 (m, 2H), 2.26-2.20 (m, 2H), 1.52-1.40 (m, 2H), 1.35 (s, 9H), 0.96-0.88 (m, 2H).

### Example 55I

### 2-[(4-{2-acetylamino-2-[4-(2-trimethylsilanyl-ethoxycarbonyl)-butylcarbamoyl]-ethyl}-2-ethyl-phenyl)-tert-butoxyoxalyl-amino]-benzoic Acid Benzhydryl Ester

5-tert-butoxycarbonylamino-pentanoic acid 2-trimethylsilanyl-ethyl ester (317 mg, 1.0 mmol) was treated with 4N HCl in dioxane at room temperature for 30 minutes, then concentrated under reduced pressure. The residue, 2-{[4-(2-acetylamino-2-carboxy-ethyl)-2-ethyl-phenyl]-tert-butoxyoxalyl-amino}-benzoic benzhydryl ester (665 mg, 1.0 mmol), 2-(1H-benzotriazole-1-yl)-1,1,3,3-tetramethyluronium tetrafluoroborate (321 mg, 1.0 mmol) and diisopropylethylamine (521 ul, 3.0 mmol) in N,N-dimethylformamide (2 mL) was stirred at ambient temperature overnight, diluted with ethyl acetate and washed with aqueous NaHCO₃(1×30 mL), brine (1×30 mL), dried (MgSO₄), filtered and concentrate under reduced pressure. The residue was purified on silica gel eluting with ethyl acetate to provide of titled compound 480 mg. MS (APCI(+)) m/e 864 (M+H)⁺.

### Example 55J

### 2-({4-[2-acetylamino-2-(4-carboxy-butylcarbamoyl)-ethyl]-2-ethyl-phenyl}-tert-butoxyoxalyl-amino)-benzoic acid benzhydryl Ester

2-[(4-{2-acetylamino-2-[4-(2-trimethylsilanyl-ethoxycarbonyl)-butylcarbamoyl]-ethyl}-2-ethyl-phenyl)-tert-butoxyoxalyl-amino]-benzoic acid benzhydryl ester (356 mg, 0.41 mmol) was treated with tetrabutylammonium fluoride-1M in THF (4 mL) at room temperature for 2 hours. The mixture was diluted with ethyl acetate and washed with 1N HCl (3×25 mL), dried (MgSO₄), filtered and concentrated under reduced pressure to provide the titled compound (305 mg). MS (APCI(+)) m/e 764 (M+H)⁺; ¹H NMR (300 MHz, DMSO-d₆) δ8.31-7.90(m, 2H), 7.73-6.85 (m, 16H), 4.43-4.33 (m, 1H), 3.22-2.48 (m, 6H), 2.22-2.15 (m, 2H), 1.80-1.72 (m, 3H), 1.62-1.25 (m, 4H), 1.05, 1.04, 1.00 (s, s, s, 9H), 1.25-0.78 (m, 3H).

### Example 55K

### 2-1(4-{2-acetylamino-2-[4-(1-phenyl-ethylcarbamoyl)-butylcarbamoyl]-ethyl}-2-ethyl-phenyl)-tert-butoxyoxalyl-amino)-benzoic Acid Benzhydryl Ester

A mixture of 2-({4-[2-acetylamino-2-(4-carboxy-butylcarbamoyl)-ethyl]-2-ethyl-phenyl}-tert-butoxyoxalyl-amino)-benzoic acid benzhydryl ester (25 mg, 0.03 mmol), 1-phenyl-ethylamine (10 μL, 0.07 mmol), 2-(1H-benzotriazole-1-yl)-1,1,3,3-tetramethyluronium tetrafluoroborate (12 mg, 0.036 mmol) and diisopropylethylamine (20 in N,N-dimethylformamide (250 μL) was stirred at ambient temperature overnight, concentrated under reduced pressure and the residue purified by reverse-phase HPLC eluting with 5-100% acetonitrile/aqueous 0.1% TRIFLUOROACETIC ACID to provide the titled compound.

### Example 55L

### N-acetyl-4-[(carboxycarbonyl)(2-carboxyphenyl)amino]-3-ethyl-N-{5-oxo-5-[1-phenylethyl)amino]pentyl}phenylalaninamide

The material from Example 55K was treated with trifluoroacetic acid/dichloromethane (10 mL, 1:1) at ambient temperature for 3 hours, concentrated under reduced pressure and purified by HPLC eluting with 5-100% acetonitrile/ aqueous 0.1% trifluoroacetic acid to provide the titled compound (8 mg). MS (ESI(+)) m/e 645 (M+H)⁺; ¹H NMR (500 MHz, DMSO-d₆) δ8.20-7.79 (m, 4H), 7.56-6.75 (m, 1H), 4.93-4.88 (m, 1H), 4.50-4.40 (m, 1H), 3.04-2.97 (m, 2H), 2.98-2.89 (m, 1H), 2.80-2.71 (m, 1H), 2.70-2.55 (m, 2H), 2.12-2.06 (m, 2H), 1.77, 1.75 (s, s, 3H), 1.48-1.41 (m, 2H), 1.38-1.28 (m, 4H), 1.07, 0.93 (t, t, 3H).

### EXAMPLE 56

### N-(methoxycarbonyl)-4-[(carboxycarbonyl)(2-carboxyphenyl)amino]-N-pentylnaphthylalaninamide

### Example 56A

### 1-methyl-4-nitro-naphthalene

The titled compound was prepared according to the procedure described in J. Org. Chem. 1991, 56, 1739. Davalli, S., Lunazzi, L., Macciantelli, D.

### Example 56B

### 3-(4-nitro-1-naphthyl)alanine

The titled compound was prepared from 1-methyl-8-nitronaphthalene according to the procedure described in J. Med. Chem. 1967, 10, 293. Benigni, J. D., Minnis, R. L.

### Example 56C

### 2-methoxycarbonylamino-3-(4-nitro-naphthalen-1-yl)-propionic Acid

A mixture of 3-(4-nitro-1-naphthyl)alanine (0.65 g, 2.5 mmol), aqueous NaHCO₃ (5 mL) and methylchloroformate (230 uL, 3 mmol, 1.2 eq) in dioxane (10 mL) was stirred for 3 hours, acidified to a pH <3with aqueous 2N HCl and extracted with ethyl acetate. The combined organic layers was washed with water (1×25 mL), brine (1×25 mL), dried (MgSO₄), filtered and concentrated under reduce pressure to provide the titled compound. MS (APCI(+)) m/e 319 (M+H)⁺.

### Example 56D

### 2-methoxycarbonylamino-3-(4-nitro-naphthalen-1-yl)-propionic acid 2-trimethylsilanyl-ethyl Ester

To a mixture of 2-methoxycarbonylamino-3-(4-nitro-naphthalen-1-yl)-propionic acid (0.35 g, 1.1 mmol), pyridine (0.78 mL) and 2-trimethylsilylethanol (0.18 mL, 1.25 mmol, 1.1 eq) in acetonitrile (1.1 mL) cooled in an ice bath was added dicyclohexylcarbodiimide (0.25 g, 1.21 mmol). The mixture was stirred cold for 1 hour, placed in the refrigerator for 14 hours. The reaction mixture was filtered, concentrated under reduced pressure and purified on silica gel eluting with heptane/ethyl acetate (4:1) to provide the titled compound. MS (ESI(−)) m/e 417 (M−H)⁻.

### Example 56E

### 3-(4-amino-naphthalen-1-yl)-2-methoxycarbonylamino-propionic acid 2-trimethylsilanyl-ethyl Ester

A mixture of 2-methoxycarbonylamino-3-(4-nitro-naphthalen-1-yl)-propionic acid 2-trimethylsilanyl-ethyl ester (1.1 g, 2.64 mmol), 10% palladium on C (0.056 g) in methanol (5 mL) was stirred under an atmosphere of hydrogen for 4 hours. The mixture was filtered through diatomaceous earth and the filter cake washed with methanol (2×25 mL). The combined methanol was concentrated under reduced pressure to provide the titled compound. MS (ESI(+)) m/e 389 (M+H)⁺.

### Example 56F

### 2-{4-[2-methoxycarbonylamino-2-(2-trimethylsilanyl-ethoxycarbonyl)-ethyl]-naphthalen-1-ylamino}-benzoic Acid

A mixture of 3-(4-amino-naphthalen-1-yl)-2-methoxycarbonylamino-propionic acid 2-trimethylsilanyl-ethyl ester (0.93 g, 2.40 mmol), diphenyliodonium-2-carboxylate (1.22 g, 3.8 mmol, 1.5 eq) and copper(II) acetate (25 mg, 0.14 mmol, 0.06 eq) in N,N-dimethylformamide (25 mL) was heated to 100° C. for 14 hours, then cooled to room temperature. The mixture was acidified to a pH <3with 1N HCl, extracted with ethyl acetate (3×35 mL). The combined organic layers were washed with 1N HCl (1×25 mL), water (1×25 mL), brine (1×25 mL), and dried (MgSO₄), filtered and concentrated under reduced pressure. The residue was purified on silica gel eluting with 4:1 toluene/ethyl acetate to provide the titled compound. MS (ESI(−)) m/e 507 (M−H)⁻.

### Example 56G

### 2-(tert-butoxyoxalyl-{4-[2-methoxycarbonylamino-2-(2-trimethylsilanyl-ethoxycarbonyl)-ethyl]-naphthalen-1-yl}-amino)-benzoic Acid

To a mixture of 2-{4-[2-methoxycarbonylamino-2-(2-trimethylsilanyl-ethoxycarbonyl)-ethyl]-naphthalen-1-ylamino}-benzoic acid (0.7 g, 1.38 mmol) and diisopropylethylamine (0.57 mL) in methylene chloride (8 mL) at 0° C. was slowly added tert-butyl oxalyl chloride (538 mg, 3.61 mmol, 2.6 eq). The reaction was allowed to warm to room temperature over 1 hour and 4-(dimethylamino)pyridine (10 mg, 0.08 mmol, 0.06 eq) was added. The reaction was stirred for 14 hours, acidified to a pH <3with 1N HCl, extracted with ethyl acetate (3×30 mL). The organic layer was washed with 1N HCl (2×30 mL), water (1×20 mL), and brine (1×20 mL), dried (MgSO₄), filtered and concentrated. The residue was purified on silica gel eluting with toluene/ethyl acetate (10:1 ) to provide the titled product. MS (APCI(+)) m/e 637 (M+H)⁺

### Example 56H

### 2-(tert-butoxyoxalyl-{4-[2-methoxycarbonylamino-2-(2-trimethylsilanyl-ethoxycarbonyl)-ethyl]-naphthalen-1-yl}-amino)-benzoic Acid Benzhydryl Ester

Diphenyldiazomethane was prepared according to the procedure described in J. Org. Chem. 1959, 24, 560, Miller, J. B.

To a mixture of 2-(tert-butoxyoxalyl-{4-[2-methoxycarbonylamino-2-(2-trimethylsilanyl-ethoxycarbonyl)-ethyl]-naphthalen-1-yl}-amino)-benzoic acid (0.3 g, 0.47 mmol) in acetone (3 mL) was added diphenyldiazomethane (134 mg, 0.69 mmol). The reaction mixture was stirred for 6 hours, acidified to a pH <3 with 1N HCl and extracted with ethyl acetate (3×20 mL). The organic layer was washed with 1N HCl (1×20 mL), water (2×15 mL), brine (1×30 mL), dried (MgSO₄), filtered and concentrated under reduced pressure. The concentrate was purified on silica gel eluting with 10:1 toluene/ethyl acetate to provide the titled product. MS (ESI(+)) m/e 820 (M+H₂O+H)⁺

### Example 56I

### 2-{tert-butoxyoxalyl-[4-(2-carboxy-2-methoxycarbonylamino-ethyl)-naphthalen-1-yl]-amino}-benzoic Acid Benzhydryl Ester

To 2-(tert-butoxyoxalyl-{4-[2-methoxycarbonylamino-2-(2-trimethylsilanyl-ethoxycarbonyl)-ethyl]-naphthalen-1-yl}-amino)-benzoic acid benzhydryl ester (0.7 g, 0.87 mmol) in tetrahydrofuran (2.5 mL) cooled in an ice bath was added tetrabutylammonium fluoride (1.5 mL, 1M in tetrahydrofuran). The mixture was stirred at 0° C. for 1 hour, ambient temperature for 1 hour, diluted with 1N HCl (40 mL) and extracted with methylene chloride ( 3×30 mL). The combined organic layers were washed with 1N HCl (2×20 mL), water (1×20 mL), brine ( 2×20 mL), dried (MgSO₄), filtered and concentrated under reduced pressure. The residue was purified on silica gel eluting with 10:1 toluene/ethyl acetate to provide the titled product. MS (ESI(+)) m/e 720 (M+H₂O+H)⁺

### Example 56J

### N-(methoxycarbonyl)-4-[(carboxycarbonyl)(2-carboxyphenyl)amino]-N-pentylnaphthylalaninamide

2-{tert-butoxyoxalyl-[4-(2-carboxy-2-methoxycarbonylamino-ethyl)-naphthalen-1-yl]-amino}-benzoic acid benzhydryl ester (1.053 g, 1.5 mmol) was dissolved in 1,2-dichloroethane (15 mL) to yield a 0.1 M stock solution. I -hydroxy-7-azabenzotriazole (0.23 g, 1.69 mmol) was dissolved in 20% N,N-dimethylacetamide in 1,2-dichloroethane (10 mL) to yield a 0.17 M stock solution. N-cyclohexylccarbodiimide, N′-methyl polystyrene HL resin (Nova Biochem; 35 mg per well, f=2 mmol/g, 0.070 mmol) was added to the wells of a Robbins reaction block, followed by 0.35 mL of the 0.17 M 1-hydroxy-7-azabenzotriazole solution (0.060 mmol) and 0.5 mL of the 0.1M compound 2-{tert-butoxyoxalyl-[4-(2-carboxy-2-methoxycarbonylamino-ethyl)-naphthalen-1-yl]-amino}-benzoic acid benzhydryl ester solution (0.050 mmol). After shaking the block for one hour, 0.35 mL of a 0.1 M stock solution of 1-pentylamine (0.035 mmol) in N,N-dimethylacetamide was added to the first well and the block was shaken for 14 hours. Unreacted acid (2-{tert-butoxyoxalyl-[4-(2-carboxy-2-methoxycarbonylamino-ethyl)-naphthalen-1-yl]-amino}-benzoic acid benzhydryl ester) was removed by addition of tris-(2-aminoethyl)-amine polystyrene HL resin (Nova Biochem; 35 mg per well, f=2.3 mmol/g, 0.080 mmol). After shaking the block for 2 hours, the reaction mixture was filtered through the frit of the Robbins reaction block into a collection block and the resin was washed with 1,2-dichloroethane. The filtrate in the collection block was concentrated under vacuum. The protecting groups were removed by treatment with a solution consisting of 1,2-dichloroethane: trifluoroacetic acid: anisole (50%, 45%, 5%, 1 mL) for 3 hours. The mixtures were concentrated under reduced pressure and the crude product was purified by preparative reverse-phase HPLC. MS (APCI(+)) m/e 506 (M+H-CO₂)⁺, 550 (M+H)⁺; ¹H NMR (500 MHz, DMSO-d₆) δ8.50-6.75 (m, 13H), 4.40-4.25 (m, 1H), 3.10-2.95 (m, 2H), 1.40-1.10 (m, 6H), 0.90-0.75 (m, 3H), (signals of methylcarbamate and benzylic protons appeared in H₂O signal).

### EXAMPLE 57

### 4-[(carboxycarbonyl)(2-carboxyphenyl)amino]-N-(cyclohexylmethyl)-N-(methoxycarbonyl)naphthylalaninamide

The titled compound was prepared according to the method described in Example 56 J by substituting cyclohexylmethylamine for 1-pentylamine. MS (APCI(+)) m/e 532 (M+H—CO₂)⁺, 576 (M+H)⁺; ¹H NMR (500 MHz, DMSO-d₆) δ8.50-6.75 (m, 13H), 4.45-4.25 (m, 1H), 3.00-2.80 (m, 2H), 1.70-0.95 (m, 9H), 0.80 (m, 2H), (signals of methylcarbamate and benzylic protons appeared in H₂O signal).

### EXAMPLE 58

### 4-[(carboxycarbonyl)(2-carboxyphenyl)amino]-N-(methoxycarbonyl)-N-[(1R)-1-(4-nitrophenyl)ethyl]naphthylalaninamide

The titled compound was prepared according to the method described in Example 56 J by substituting (1R)-1-(4-nitrophenyl)ethylamine for 1-pentylamine. MS (APCI(+)) m/e 585 (M+H-CO₂)⁺, 629 (M+H)⁺; ¹H NMR (500 MHz, DMSO-d₆) δ8.75-6.75 (m, 17H), 5.05-4.80 (m, 1H), 4.50-4.35 (m, 1H), 1.45-0.95 (m, 3H), (signals of methylcarbamate and benzylic protons appeared in H₂O signal).

### EXAMPLE 59

### 4-[(carboxycarbonyl)(2-carboxyphenyl)amino]-N-(methoxycarbonyl)-N-[4-(methylsulfonyl)benzyl]naphthylalaninamide

The titled compound was prepared according to the method described in Example 56 J by substituting 4-(methylsulfonyl)benzylamine for 1-pentylamine. MS (APCI(+)) m/e 604 (M+H—CO₂)⁺, 648 (M+H)⁺; ¹H NMR (500 MHz, DMSO-d₆) δ8.75-6.75 (m, 17H), 4.50-4.30 (m, 3H), 3.20-3.15 (m, 3H), (signals of methylcarbamate and benzylic protons appeared in H₂O signal).

### EXAMPLE 60

### 4-[(carboxycarbonyl)(2-carboxyphenyl)amino]-N-(methoxycarbonyl)-N-(3,4,5-trifluorobenzyl)naphthylalaninamide

The titled compound was prepared according to the method described in Example 56 J by substituting 3,4,5-trifluorobenzylamine for 1-pentylamine. MS (APCI(+)) m/e 580 (M+H—CO₂)⁺, 624 (M+H)⁺; ¹H NMR (500 MHz, DMSO-d₆) δ8.75-6.75 (m, 15H), 4.45-4.25 (m, 2H), 4.25-4.15 (m, 1H), (signals of methylcarbamate and benzylic protons appeared in H₂O signal).

### EXAMPLE 61

### 4-[(carboxycarbonyl)(2-carboxyphenyl)amino]-N-(cyclooctylmethyl)-N-(methoxycarbonyl)naphthylalaninamide

The titled compound was prepared according to the method described in Example 56 J by substituting cyclooctylmethylamine for 1-pentylamine. MS (APCI(+)) m/e 560 (M+H—CO₂)⁺, 604 (M+H)⁺; ¹H NMR (500 MHz, DMSO-d₆) δ8.50-6.75 (m, 13H), 4.45-4.25 (m, 1H), 2.95-2.8 (m, 2H), 1.7-1.0 (m, 15H), (signals of methylcarbamate and benzylic protons appeared in H₂O signal)

### EXAMPLE 62

### 4-[(carboxycarbonyl)(2-carboxyphenyl)amino]-N-[(1R)-1-(4-bromophenyl)ethyl]-N-(methoxycarbonyl)naphthylalaninamide

The titled compound was prepared according to the method described in Example 56 J by substituting (1R)-1-(4-bromophenyl)ethylamine for 1-pentylamine. MS (APCI(+)) m/e 618 (M+H—CO₂)⁺, 662 (M+H)⁺; ¹H NMR (500 MHz, DMSO-d₆) δ8.65-6.75 (m, 17H), 4.95-4.70 (m, 1H), 4.45-4.30 (m, 1H), 1.40-0.95 (m, 3H), (signals of methylcarbamate and benzylic protons appeared in H₂O signal).

### EXAMPLE 63

### 4-[(carboxycarbonyl)(2-carboxyphenyl)amino]-N-(methoxycarbonyl)-N-(3-phenylpropyl)naphthylalaninamide

The titled compound was prepared according to the method described in Example 56 J by substituting 3-phenylpropylamine for 1-pentylamine. MS (APCI(+)) m/e 554 (M+H—CO₂)⁺, 598 (M+H)⁺; ¹H NMR (500 MHz, DMSO-d₆) δ8.50-6.75 (m, 18H), 4.4-4.25 (m, 1H), 3.15-3.00 (m, 2H), 1.75-1.5 (m, 2H), (signals of methylcarbamate and benzylic protons appeared in H₂O signal).

### EXAMPLE 64

### Methyl 3-{4-[(carboxycarbonyl)(2-carboxyphenyl)amino]-1-naphthyl}-N-(methoxycarbonyl)alanyl-L-norleucinate

The titled compound was prepared according to the method described in Example 56 J by substituting L-methyl-norleucinate for 1-pentylamine. MS (APCI(+)) m/e 564 (M+H—CO₂)⁺, 608 (M+H)⁺; ¹H NMR (500 MHz, DMSO-d₆) δ8.50-6.75 (m, 13H), 4.55-4.40 (m, 1H), 4.35-4.15 (m, 1H), 3.65-3.55 (m, 3H), 1.8-1.0 (m, 6H), 0.85-0.65 (m, 3H), (signals of methylcarbamate and benzylic protons appeared in H₂O signal).

### EXAMPLE 65

### 4-[(carboxycarbonyl)(2-carboxyphenyl)amino]-N-(2-fluorobenzyl)-N-(methoxycarbonyl)naphthylalaninamide

The titled compound was prepared according to the method described in Example 56 J by substituting 2-fluorobenzylamine for 1-pentylamine. MS (APCI(+)) m/e 544 (M+H—CO₂)⁺, 588 (M+H)⁺; ¹H NMR (500 MHz, DMSO-d₆) δ8.70-6.75 (m, 17H), 4.50-4.20 (m, 3H), (signals of methylcarbamate and benzylic protons appeared in H₂O signal).

### EXAMPLE 66

### 4-[(carboxycarbonyl)(2-carboxyphenyl)amino]-N-(4-chlorobenzyl)-N-(methoxycarbonyl)naphthylalaninamide

The titled compound was prepared according to the method described in Example 56 J by substituting 4-chlorobenzylamine for 1-pentylamine. MS (APCI(+)) m/e 560 (M+H—CO₂)⁺, 604 (M+H)⁺; ¹H NMR (500 MHz, DMSO-d₆) δ8.75-6.75 (m, 17H), 4.50-4.35 (m, 1H), 4.35-4.15 (m, 2H), (signals of methylcarbamate and benzylic protons appeared in H₂O signal).

### EXAMPLE 67

### 4-[(carboxycarbonyl)(2-carboxyphenyl)amino]-N-(4-bromobenzyl)-N-(methoxycarbonyl)naphthylalaninamide

The titled compound was prepared according to the method described in Example 56 J by substituting 4-bromobenzylamine for 1-pentylamine. MS (APCI(+)) m/e 604 (M+H—CO₂)⁺, 648 (M+H)⁺; ¹H NMR (500 MHz, DMSO-d₆) δ8.70-6.75 (m, 17H), 4.50-4.35 (m, 1H), 4.35-4.15 (m, 2H), (signals of methylcarbamate and benzylic protons appeared in H₂O signal).

### EXAMPLE 68

### 4-[(carboxycarbonyl)(2-carboxyphenyl)amino]-N-(methoxycarbonyl)-N-(4-nitrobenzyl)naphthylalaninamide

The titled compound was prepared according to the method described in Example 56 J by substituting 4-nitrobenzylamine for 1-pentylamine. MS (APCI(+)) m/e 571 (M+H—CO₂)⁺, 615 (M+H)⁺; ¹H NMR (500 MHz, DMSO-d₆) δ8.85-6.75 (m, 17H), 4.50-4.30 (m, 3H), (signals of methylcarbamate and benzylic protons appeared in H₂O signal).

### EXAMPLE 69

### 4-[(carboxycarbonyl)(2-carboxyphenyl)amino]-N-[4-(aminosulfonyl)benzyl]-N-(methoxycarbonyl)naphthylalaninamide

The titled compound was prepared according to the method described in Example 56 J by substituting 4-(aminosulfonyl)benzylamine for 1-pentylamine. MS (APCI(+)) m/e 605 (M+H—CO₂)⁺, 649 (M+H)⁺; ¹H NMR (500 MHz, DMSO-d₆) δ8.90-6.75 (m, 19H), 4.50-4.25 (m, 3H), (signals of methylcarbamate and benzylic protons appeared in H₂O signal).

### EXAMPLE 70

### 4-[(carboxycarbonyl)(2-carboxyphenyl)amino]-N-(methoxycarbonyl)-N-({4-[(methylamino)carbonyl]cyclohexyl}methyl)naphthylalaninamide

The titled compound was prepared according to the method described in Example 56 J by substituting trans-4-[(methylamino)carbonyl]cyclohexylmethylamine for 1-pentylamine. MS (APCI(+)) m/e 589 (M+H—CO₂)⁺, 633 (M+H)⁺; ¹H NMR (500 MHz, DMSO-d₆) δ8.50-6.75 (m, 13H), 4.45-4.25 (m, 1H), 3.00-2.80 (m, 2H), 2.56-2.53(m, 3H), 2.05-1.90 (m, 1H), 1.75 -1.50 (m, 3H), 1.40-1.15 (m, 3H), 0.90-0.70 (m, 2H), (signals of methylcarbamate and benzylic protons appeared in H₂O signal).

### EXAMPLE 71

### N-acetyl-4-[(carboxycarbonyl)(2-carboxyphenyl)amino]-3-(2-hydroxyethyl)-N-(4-nitrobenzyl)phenylalaninamide

The titled compound was prepared according to the procedure described in Example 35 C-G, substituting the 4-nitro-benzylamine for the amylamine, and 2-acetylamino-3-[4-amino-3-(2-hydroxy-ethyl)-phenyl]-propionic acid for 2-acetylamino-3-(4-amino-naphthalen-1-yl)-propionic acid used in Example 35D. MS (ESI+) m/e 593 (M+H)⁺; ¹H NMR (300 MHz, DMSO-d₆) (A mixture of rotamers) d 8.65 (brt, J=5.7 Hz, 1H), 8.26-8.07 (m, 2H), 7.91 and 7.82 (two sets of doublets, J=7.2 Hz, 1H in total), 7.60-6.62 (m, 9H), 4.63-4.27 (m, 3H), 3.85-3.10 (m, 2H), 3.10-2.64 (m, 4H), 1.85-1.75 (multiple siglets, 3H in total).

### EXAMPLE 72

### 2-[(carboxycarbonyl)(7-hydroxy-1-naphthyl)amino]-4-cyanobenzoic Acid

### EXAMPLE 72A

### Methyl 2-nitro-4-carboxybenzoate

A solution of dimethyl nitroterephthalate (5.98 g, 25.0 mmol) and aqueous 1M NaOH. (25 mL) in dioxane (50 mL) was stirred at ambient temperature for 2 hours, then concentrated under reduced pressure. The remaining solution was diluted with water (50 mL) and extracted with diethyl ether (3×25 mL). The aqueous layer was acidified to a pH<3 with 1M HCl, then extracted with diethyl ether (3×25 mL). The second set of ether layers was washed with brine (1×10 mL), dried (MgSO₄), filtered, and concentrated under reduced pressure. The residue was precipitated from methanol (25 mL) and water (25 mL) to provide the titled compound (2.0 g, 36%). ¹H NMR (300 MHz, d₆-DMSO) δ8.46 (d, 1H, J=135), 8.33 (dd, 1H, J=1.7, 7.8), 7.99 (d, 1H, J=8.1), 3.89 (s, 3); MS (ESI) m/z=224 (M−H).

### Example 72B

### benzyl 3-nitro-4-methoxycarbonylbenzoate

To a solution of methyl 2-nitro-4-carboxybenzoate (1.13 g, 5.00 mmol), K₂CO₃ (680 mg, 4.92 mmol) in N,N-dimethylformamide (5 mL) was added benzyl bromide (750 μL, 6.30 mmol). The mixture was heated at 100° C. for 15 minutes, cooled to ambient temperature, poured into water (30 mL) and extracted with diethyl ether (3×10 mL). The combined ether layers were washed with water (2×10 mL), brine (1×10 mL), dried (MgSO₄), filtered, and concentrated under reduced pressure. The residue was crystallized from 20% ethyl acetate/hexanes to provide the titled compound (1.0 g, 63%). ¹H NMR (300 MHz, d₆-DMSO) δ8.52 (d, 1, J=1.7), 8.39 (dd, 1, J=1.7, 8.1), 8.01 (d, 1, J=7.8), 7.53-7.35 (m, 5), 5.43(s, 2), 3.89 (s, 3); MS (ESI) m/z=333 (M+NH₄⁺).

### Example 72C

### benzyl-3-amino-4-methoxycarbonylbenzoate

A solution of benzyl-3-nitro-4-methoxycarbonylbenzoate (958 mg, 3.04 mmol), iron powder (990 mg, 18 mmol), ammonium chloride (200 mg, 3.73 mmol) in 2-propanol (15 mL) and water (3 mL) was heated to reflux for 1 hour, filtered and concentrated under reduced pressure. The residue was partitioned between water (10 mL) and diethyl ether (10 mL). The ether layer was washed with brine (1×5 mL), dried (MgSO₄), filtered, and concentrated under reduced pressure to provide the titled compound (528 mg, 61%). ¹H NMR (300 MHz, d₆-DMSO) δ7.80 (d, 1H, J=8.1), 7.48-7.33(m, 6H), 7.08 (dd, 1, J=1.7, 8.1), 6.87 (bs, 2H), 5.33 (s, 2), 3.81 (s, 3); MS (ESI) m/z 286 (MH⁺).

### Example 72D

### 1-Iodo-7-hydroxynaphthalene

To 8-amino-2-naphthol (4.48 g, 30 mmol) was added a solution of 98% H₂SO₄ (4 mL) in water (50 mL). The mixture was cooled with an ice bath, then a solution of aqueous NaNO₂ (2.50 g, 36.2 mmol) in a minimum amount of water was added slowly via pipette, expelling the nitrite solution below the surface of the reaction. After 10 min, a solution of KI (15 g, 90 mmol) in a minimum amount of water was added slowly to control the evolution of N₂. Diethyl ether was added to break the foamy emulsion that formed and the reaction was stirred for 18 hour at ambient temperature. Additional diethyl ether (50 mL) was added, followed by solid NaHSO₃ to remove the 12 color from the reaction. The mixture was filtered and the filter cake washed with ethyl acetate (150 mL). The ether layer was separated from the supernatant, and the aqueous layer was extracted with diethyl ether (1×50 mL). The combined ether and ethyl acetate layers were washed with brine (1×25 mL), dried (MgSO₄), filtered, and concentrated under reduced pressure and the residue purified on silica gel eluting with 20% ethyl acetate/hexanes to provide the titled compound (3.2 g, 40%). ¹H NMR (300 MHz, d₆-DMSO) δ10.12 (s, 1H), 8.02 (dd, 1H, J=1.2, 7.3), 7.84 (d, 1H, J=8.1), 7.78 (d, 1H, J=8.8), 7.33 (d, 1H, J=2.4), 7.13 (dd, 1H, J=2.4, 8.8), 7.04 (dd, 1H, J=7.1, 8.1); MS (ESI) m/z=269 (M−H).

### Example 72E

### 1-Iodo-7-dimethyl-tert-butylsilyloxynaphthalene

To 1-iodo-7-hydroxynaphthalene (3.05 g, 11.3 mmol) in N,N-dimethylformamide (27 mL) was added tert-butyldimethylsilyl chloride (2.09 g, 13.9 mmol) and imidazole (1.85 g, 27 mmol). The reaction was stirred at ambient temperature for 2 hours, diluted with aqueous 0.3M HCl (150 mL) and extracted with hexanes (2×50 mL). The combined hexanes layers were washed with water (1×50 mL), brine (1×50 mL), dried (MgSO₄), filtered, and concentrated under reduced pressure. The residue was heated at 110° C. for 2 min to remove most of the excess silicon containing byproducts to provide the titled compound (4.34 g, 100%). ¹H NMR (300 MHz, d₆-DMSO) δ8.01 (dd, 1H, J=1.0, 7.1), 7.75 (d, 1H, J=8.1), 7.49 (d, 1H, J=2.4), 7.09 (dd, 1H, J=2.4, 8.8), 7.04 (dd, 1H, J=7.5, 7.8), 1.04 (s, 9H), 0.31 (s, 6H); MS (ESI) m/z=269 (M-TBS).

### Example 72F

### 2-[7-(tert-butyl-dimethyl-silanyloxy)-naphthalen-1-ylaminol-terephthalic Acid 4-benzyl Ester 1-methyl Ester

A mixture of benzyl-3-amino-4-methoxycarbonylbenzoate (1.2 g, 4.21 mmol), 1-iodo-7-dimethyl-tert-butylsilyloxynaphthalene (1.62 g, 4.21 mmol), tris-(dibenzylideneacetone)dipalladium (19 mg, 0.019 mmol), (2′-dicyclohexylphosphanyl-biphenyl-2-yl)-dimethyl-amine (25 mg, 0.064 mmol), and 60% NaH in mineral oil (210 mg, 5.25 mmol) in toluene (13 mL) was stirred under N₂ for 17 hours at 80° C., cooled to ambient temperature and poured into aqueous 0.1M HCl (50 mL). The mixture was extracted with diethyl ether (3×15 mL), the combined ether layers were washed with brine (1×15 mL), dried (MgSO₄), filtered, and concentrated under reduced pressure. The residue was purified on silica gel eluting with 10% ethyl acetate: hexanes to provide the titled compound (1.26 g, 55%). ¹H NMR (300 MHz, CDCl₃) δ9.70 (bs, 1H), 8.05 (d, 1H, J=8.5), 7.79 (d, 1H, J=8.8), 7.68 (d, 1H, J=8.1), 7.55 (d, 1H, J=1.7), 7.46 (d, 1H, J=7.5), 7.35-7.24 (m, 8), 7.10 (dd, 1H, J=2.4, 8.8), 5.23(s, 2H), 3.98 (s, 3H), 1.04 (s, 9H), 0.12 (s, 6H); MS (ESI) m/z=542 (MH⁺).

### Example 72G

### 2-(7-hydroxy-naphthalen-1-ylamino)-terephthalic Acid 4-benzyl Ester 1-methyl Ester

To 2-[7-(tert-Butyl-dimethyl-silanyloxy)-naphthalen-1-ylamino]-terephthalic acid 4-benzyl ester 1-methyl ester (1.26 g) in dioxane (30 mL) was added 15 drops of 60% HClO₄. After stirring for 18 hours, aqueous NaHCO₃solution (2 mL) was added, the mixture was concentrated under reduced pressure and the residue partitioned between ethyl acetate (15 mL) and water (5 mL). The ethyl acetate layer was washed with brine (1×5 mL), dried (MgSO₄), filtered, and concentrated under reduced pressure. The residue was purified on silica gel eluting with ethyl acetate:hexanes to provide the titled compound (553 mg, 56%). ¹H NMR (300 MHz, d₆-DMSO) δ9.84 (s, 1H), 9.50 (s, 1H), 8.05 (m, 1H), δ7.89 (m, 1H), 7.78 (d, 1H, J=8.1), 7.45 (d, 1H, J=7.1), 7.37-7.24 (m, 8), 7.15-7.12 (m, 2H), 5.21 (s, 2H), 3.95 (s, 3H); MS (ESI) m/z=426 (M−H).

### Example 72H

### 2-(7-hydroxy-naphthalen-1-ylamino)-terephthalic Acid 1-methyl Ester

A mixture containing 2-(7-hydroxy-naphthalen-1-ylamino)-terephthalic acid 4-benzyl ester 1-methyl ester (545 mg, 1.27 mmol), 10% Pd—C (90 mg) and 60% HClO₄ (2 drops) in dioxane (10 mL ) was stirred at ambient temperature under 1 atmosphere hydrogen for 4 hours. The mixture was filtered, aqueous NaHCO₃ solution (1 mL) was added, and concentrated under reduced pressure. The residue was partitioned between ethyl acetate (15 mL) and aqueous 1M HCl (5 mL), and the layers separated. The organic layer was washed with brine (1×5 mL), dried (MgSO₄), filtered, and concentrated under reduced pressure to provide the titled compound (450 mg). ¹H NMR (300 MHz, d₆-DMSO) δ13.1 (bs, 1H), 9.84 (s, 1H), 9.48 (s, 1H), 8.02 (d, 1H, J=8.5), 7.87 (d, 1H, J=8.5), 7.75 (d, 1H, J=8.1), 7.43 (d, 1H, J=7.1), 7.34 (t, 1H, J=7.6), 7.24 (dd, 1H, J=1.7, 8.1), 7.21 (d, 1H, J=1.7), 7.16-7.11 (m, 2H), 3.95 (s, 3H); MS (ESI) m/z=336 (M−H).

### Example 72I

### 2-(7-hydroxy-naphthalen-1-ylamino)-terephthalyl amide 1-methyl Ester

To a solution of 2-(7-hydroxy-naphthalen-1-ylamino)-terephthalic acid 1-methyl ester (75 mg, 0.22 mmol) in THF (2 mL ) was added triethylamine (155 μL, 1.11 mmol) and isobutyl chloroformate (80 μL, 0.62 mmol). The reaction was stirred at ambient temperature for 30 min, aqueous ammonia (1 mL) was added, the reaction was stirred for 5 hours and concentrated under reduced pressure. The residue was taken up in ethyl acetate (3 mL ), washed with 1M HCl (3×1 mL), aqueous NaHCO₃solution (2×1 mL), brine (1×1 mL), dried (MgSO₄), filtered, and concentrated under reduced pressure. The residue was purified on silica gel eluting with ethyl acetate: hexanes to provide the titled compound.

### Example 72J

### 2-[(carboxycarbonyl)(7-hydroxy-1-naphthyl)amino]-4-cyanobenzoic Acid

A mixture of 2-(7-hydroxy-naphthalen-1-ylamino)-terephthalyl amide 1-methyl ester and 1.39M NaOH (1 mL) in 5:1 ethanol: water was stirred at ambient temperature for 2 hours, and concentrated under reduced pressure. The residue was partitioned between water (1 mL) and ethyl acetate (2 mL), the mixture was acidified to a pH <3with aqueous 1 M HCl (2 mL). The layers were separated, and the aqueous layer was extracted with ethyl acetate (2 mL). The combined ethyl acetate layers were washed with brine (1×2 mL), dried (MgSO₄), filtered, and concentrated under reduced pressure. A solution of the residue, triethylamine (200 μL) and ethyl oxalyl chloride (100 μL) in N,N-dimethylformamide (1 mL) was stirred at ambient temperature for 40 minutes, then aqueous 2M NaOH (2 mL) was added, followed by the addition of water (6 mL). The resulting solution was stirred for 10 minutes, then ethyl acetate (2 mL) was added, followed by 12M HCl (0.5 mL).. The mixture was shaken, then separated, and the aqueous phase was extracted with ethyl acetate (2×1 mL). The combined ethyl acetate layers were dried (MgSO₄), filtered, and concentrated under reduced pressure. The residue was purified on reverse phase HPLC, eluting with a 0.1% aqueous trifuoroacetic acid:acetonitrile to provide the titled compound (18 mg, 23%). ¹H NMR (500 MHz, d₆-DMSO) mixture of rotamers, major rotamer resonances only, δ9.93 (bs, 1H), 8.03 (d, 1H, J=8.1), 7.92 (d, 1H, J=8.1), 7.89 (d, 1H, J=7.8), 7.87-7.83 (m, 2H), 7.64 (d, 1H, J=6.6), 7.48 (d, 1H, J=2.2), 7.36 (dd, 1H, J=7.5, 8.1), 7.28 (d, 1H, J=1.3), 7.13 (dd, 1H, J=2.3, 8.9); MS (ESI) m/z=375 (M−H), 394 (M+NH4⁺).

### EXAMPLE 73

### 4-[(carboxycarbonyl)(2-carboxyphenyl)amino]-N-{4-[(ethylamino)sulfonyl]benzyl}-N-(methoxycarbonyl)naphthylalaninamide

### Example 73A

### 4-(1,3-dioxo-1,3-dihydro-isoindol-2-ylmethyl)-benzenesulfonyl Chloride

The titled compound was prepared according to the procedure described in Bergeim, F. H.; Braker, W. J. Am. Chem. Soc. 1944, 64, 1459.

### Example 73B

### 4-(1,3-dioxo-1,3-dihydro-isoindol-2-ylmethyl)-N-ethyl benzenesulfonamide

To a mixture of 4-(1,3-dioxo-1,3-dihydro-isoindol-2-ylmethyl)-benzenesulfonyl chloride (1.68 g, 5 mmol) in dioxane (15 mL) at 0° C. was added aqueous sodium bicarbonate (5 mL) followed by ethylamine (10 mmol, 5 mL of a 2M solution in tetrahydrofuran). The mixture was stirred for 30 minutes at 0° C., then warmed to ambient temperature for 2 hours. The mixture was partitioned between water and ethyl acetate. The organic layer was washed with water (1×20 mL), aqueous 1N HCl (2×20 mL), aqueous NaHCO₃(2×20 mL), and brine (1×20 mL), dried (MgSO₄), filtered and concentrated under reduced pressure. The residue was dissolved in a minimum of chloroform and precipitated by the addition of hexane. The mixture was filtered and the solid washed with hexane and chloroform to provide the titled product. MS (APCI(+)) m/e 345 (M+H)⁺.

### Example 73C

### 4-[(ethylamino)sulfonyl]benzylamine

A solution containing 4-(1,3-dioxo-1,3-dihydro-isoindol-2-ylmethyl)-N-ethyl benzenesulfonamide (0.84 g, 2.44 mmol) and hydrazine (0.38 mL) in methanol (5 mL) was stirred under nitrogen for 14 hours diluted with aqueous NaHCO₃ and extracted with ethyl acetate. The organic layer was washed with aqueous NaHCO₃(1×35 mL), brine (1×30 mL), dried ( MgSO₄), filtered and concentrated under reduced pressure. The residue was purified on silica gel eluting with 1:1 toluene/ethyl acetate with a gradient of methanol to provide the titled compound. MS (APCI(+)) m/e 215 (M+H)⁺.

### Example 73D

### 4-[(carboxycarbonyl)(2-carboxyphenyl)amino]-N-{4-[(ethylamino)sulfonyl]benzyl}-N-(methoxycarbonyl)naphthylalaninamide

The titled compound was prepared according to the procedure described in 56 J by substituting 4-[(ethylamino)sulfonyl]benzylamine for 1-pentylamine. MS (ESI(+)) m/e 676 (M)⁺; ¹H NMR (500 MHz, DMSO-d₆) δ8.80-6.75 (m, 18H), 4.55-4.25 (m, 3H), 2.85-2.65 (m, 2H), 1.00-0.90 (m, 3H), (signals of methylcarbamate and benzylic protons appeared in H₂O signal).

### EXAMPLE 74

### N-(tert-butoxycarbonyl)-L-phenylalanyl-3-[(1E)-3-amino-3-oxo-1-propenyl]-4-[(carboxycarbonyl)(2-carboxyphenyl)amino]-N-pentyl-L-phenylalaninamide

The titled compound was prepared according to the procedure described for Example 38 C-E, substituting {1-[2-(4-amino-3-iodo-phenyl)-1-pentylcarbamoyl-ethylcarbamoyl]-2-phenyl-ethyl}carbamic acid tert-butyl ester from Example 49B for [2-(4-amino-3-iodo-phenyl)-1-pentylcarbamoyl-ethyl]-carbamic acid tert-butyl ester from Example 38B. MS (ESI+) m/e 780 (M+Na)⁺; 758 (M+H); No NMR data available.

### EXAMPLE 75

### N-acetyl-4-[(carboxycarbonyl)(2-carboxyphenyl)amino]-3-(2-hydroxyethyl)-N-[(1S)-1-(4-nitrophenyl)ethyl]phenylalaninamide

The titled compound was prepared according to the procedure described in Example 35D-G, substituting 2-acetylamino-3-[4-amino-3-(2-hydroxy-ethyl)-phenyl]-propionic acid for 2-acetylamino-3-(4-amino-naphthalen-1-yl)-propionic acid and substituting 4-nitro-(S)-α-methyl-benzylamine for the amylamine in Example 35D. MS (ESI+) m/e 607 (M+H)⁺; ¹H NMR (300 MHz, DMSO-d₆) (A mixture of rotamers and 1:1 mixture of diastereoisomers) δ8.74-8.44 (m, 2H), 8.22-7.79 (m, 4H), 7.59-6.84 (m, 7H), 5.02-4.82 (m, 1H), 4.67-4.44 (m, 1H), 3.58-3.33 (m, 2H), 3.01-2.60 (m, 4H), 1.78 (multiple s, 3H), 1.43-1.10 (m, 3H).

### EXAMPLE 76

### N-acetyl-4-[(carboxycarbonyl)(2-carboxyphenyl)amino]-N-(4-chlorobenzyl)-3-(2-hydroxyethyl)phenylalaninamide

The titled compound was prepared according to the procedure described in Example 35D-E, substituting 2-acetylamino-3-[4-amino-3-(2-hydroxy-ethyl)-phenyl]-propionic acid for 2-acetylamino-3-(4-amino-naphthalen-1-yl)-propionic acid and substituting 4-chloro-benzylamine for the amylamine in Example 35D. MS (ESI+) m/e 582, 584 (M+H)⁺; ¹H NMR (300 MHz, DMSO-d₆) (A mixture of rotamers) δ8.52 (t, J=5.25 Hz, 1H), 8.23-8.11 (m, 1H), 8.01-7.92 (m, 1H), 7.87-7.77 (m, 1H), 7.59-6.72 (m, 9H), 4.60-4.41 (m, 2H), 4.10-4.28 (m, 2H), 3.82-3.70 (m, 1H), 2.62-3.14 (m, 4H), 1.33-1.23 (m, 3H).

### EXAMPLE 77

### N-acetyl-4-[(carboxycarbonyl)(2-carboxyphenyl)amino]-N-(4-bromobenzyl)-3-(2-hydroxyethyl)phenylalaninamide

The titled compound was prepared according to the procedure described in Example 35D-G, substituting 2-acetylamino-3-[4-amino-3-(2-hydroxy-ethyl)-phenyl]-propionic acid for 2-acetylamino-3-(4-amino-naphthalen-1-yl)-propionic acid and 4-bromobenzylamine for the amylamine in Example 35D. MS (ESI+) m/e 626, 628 (M+H)⁺; ¹H NMR (300 MHz, DMSO-d₆) (A mixture of rotamers) δ8.65 (t, J=5.25 Hz, 1H), 8.35-8.14 (m, 1H), 8.11-7.97 (m, 1H), 7.89-7.77 (m, 1H), 7.65-6.82 (m, 9H), 4.60-4.41 (m, 2H), 4.10-4.28 (m, 2H), 3.82-3.70 (m, 1H), 2.62-3.14 (m, 4H), 1.33-1.23 (m, 3H).

### EXAMPLE 78

### 2-[(carboxycarbonyl)(7-hydroxy-1-naphthyl)amino]-4-{[4-(dimethylamino)benzoyl]amino}benzoic Acid

### Example 78A

### 2-[7-(tert-butyl-dimethyl-silanyloxy)-naphthalen-1-ylamino]-4-nitro-benzoic Acid Methyl Ester

A mixture of methyl 2-bromo-4-nitrobenzoate (2.86 g, 11.0 mmol), 7-tert-butyl-dimethylsilanlyoxy-1-naphthylamine (3.01 g, 11.0 mmol), tris(dibenzylideneacetone)dipalladium (50 mg, 0.051 mmol), (2′-dicyclohexylphosphanyl-biphenyl-2-yl)-dimethyl-amine (66 mg, 17 mmol), and 60% NaH in mineral oil (660 mg, 16.5 mmol) in toluene (35 mL) was refluxed for 2 hours under N₂, cooled and extracted with aqueous 1M HCl (1×15 mL), brine (1×15 mL). The HCl layer was extracted with diethyl ether (1×15 mL), and the ether layer washed with brine (1×10 mL). The combined ether and toluene layers were dried (MgSO₄), filtered, and concentrated under reduced pressure and the residue purified on silica gel eluting with 5% ethyl acetate: hexanes to provide the titled compound (2.58 g, 52%). ¹H NMR (300 MHz, CDCl₃) δ9.82 (bs, 1H), 8.15 (d, 1H, J=8.8), 7.81 (d, 1H, J=8.8), 7.75 (d, 1H, J=7.8), 7.51 (d, 1H, J=2.4), 7.47-7.36 (m, 3H), 7.25 (d, 1H, J=2.4), 7.12 (dd, 1H, J=2.4, 8.8), 4.01 (s, 3H), 0.94 (s, 9H), 0.13 (s, 6H); MS (ESI) m/z=451 (M−H).

### Example 78B

### 2-[7-(tert-butyl-dimethyl-silanyloxy)-naphthalen-1-ylamino]-4-amino-benzoic Acid Methyl Ester

A mixture of 2-[7-(tert-butyl-dimethyl-silanyloxy)-naphthalen-1-ylamino]-4-nitro-benzoic acid methyl ester (796 mg, 1.76 mmol), iron powder (580 mg, 10.5 mmol), ammonium chloride (116 mg, 2.16 mmol) in 2-propanol:water (8 mL of 5:1, (v/v)) was heated to reflux for 1.75 hour, diluted with ethyl acetate (50 mL), filtered through diatomaceous earth, and concentrated under reduced pressure. The product was purified on silica gel eluting with 20% ethyl acetate: hexanes to provide the titled compound (653 mg, 88%).

### Example 78C

### 2-[(carboxycarbonyl)(7-hydroxy-1-naphthyl)amino]-4-{[4-(dimethylamino)benzoyl]amino}benzoic Acid

A solution of 2-[7-(tert-butyl-dimethyl-silanyloxy)-naphthalen-1-ylamino]-4-amino-benzoic acid methyl ester (73 mg, 0.17 mmol), triethylamine (66 μL, 0.47 mmol), 4-N,N-dimethylaminobenzoyl chloride (40 mg, 0.22 mmol) in ethyl acetate (1 mL) was stirred for 10 minutes, extracted with aqueous 1M HCl (2×1 mL), aqueous NaHCO₃ solution (2×1 mL), dried (MgSO₄), filtered, concentrated under reduced pressure, and purified on silica gel eluting with 30% ethyl acetate: hexanes (37 mg). The residue was taken up and stirred in aqueous 1.39M NaOH (1 mL) in 5:1 ethanol:water for 14 hours. The mixture was concentrated under reduced pressure, taken up in water (1 mL) and extracted with diethyl ether (3×1 mL). The aqueous layer was stirred with ethyl acetate (3 mL), acidified by the addition of 2 mL of aqueous 1 M HCl. The ethyl acetate layer was separated, dried (MgSO₄), filtered, and concentrated under reduced pressure. A mixture of the residue, triethylamine (200 μL), ethyl oxalyl chloride (100 μL) in N,N-dimethylformamide (1 mL) were stirred at ambient temperature for 10 minutes, aqueous 2M NaOH (2 mL) was added and the mixture allowed to stand for 10 minutes. Water was added until a homogenous solution was achieved and the mixture was allowed to stand for 1.5 hour. The mixture was acidified to pH=1 by addition of 12M HCl, and extracted with ethyl acetate (4×1 mL). The combined ethyl acetate layers were washed with water (1×1 mL), brine (1×1 mL), dried (MgSO₄), filtered, and concentrated under reduced pressure. The residue was purified by reverse-phase HPLC, eluting with a 0.1 % trifluoroacetic acid: acetonitrile to provide the titled compound. ¹H NMR (300 MHz, d₆-DMSO) mixture of rotamers, δ10.13 (s, 1H), 9.99 (s, 1H), 7.99-7.95 (m, 2H), 7.89-7.86 (m, 2H), 7.83-7.79 (m, 2H), 7.75-7.70 (m, 2H), 7.65 (m, 1H), 7.53 (m, 1H), 7.45 (s, 1), 7.35 (m, 1H), 7.29 (d, 1H, J=2.2), 7.26 (t, 1H, J=7.8), 7.17 (m, 1H), 7.11 (dd, 1H, J=2.2, 8.7), 6.68 (m, 2H), 2.96 (s, 6H); MS (ESI) m/z=514 (MH⁺).

### EXAMPLE 79

### N-acetyl-4-[(carboxycarbonyl)(2-carboxyphenyl)amino]-3-ethyl-N-(4-nitrobenzyl)phenylalaninamide

### Example 79A

### 2-Acetylamino-acrylic Acid Benzyl Ester

To a mixture of 2-acetamidoacrylic acid (10.3 g, 80.0 mmol) and K₂CO₃(10 g, 72.5 mmol) in N,N-dimethylformamide (50 mL) was added benzyl bromide (8.7 ml, 72.5 mmol) at room temperature then stirred at room temperature for 3 hours. The mixture was partitioned between ethyl acetate and water (50 mL, 1:1), the aqueous layer was extracted with ethyl acetate (2×45 mL). The combined organic layers was washed with brine (2×25 mL), dried (MgSO₄), filtered and concentrated under reduced pressure to provide titled compound. MS (ESI(+)) m/e 220(M+H)⁺; ¹H NMR (300 MHz, DMSO-d₆) δ9.37 (s, 1H), 7.43-7.30 (m, 5H), 6.13 (s, 1H), 5.70 (s, 1H), 5.23(s, 2H), 2.01 (s, 3H).

### Example 79B

### 2-acetylamino-3-(4-amino-3-ethyl-phenyl)-acrylic Acid Benzyl Ester

To 2-acetylamino-acrylic acid benzyl ester (80.0 mmol) in acetonitrile (200 mL) was added Pd(OAc)₂ (488 mg, 2.18 mmol), (o-Tol)₃P (1.32 g, 4.35 mmol), Et₃N (20 mL) followed by addition of 4-bromo-2-ethylaniline (14.5 g, 72.5 mmol). The reaction mixture was heated to reflux overnight, concentrated under reduce pressure, taken up in ethyl acetate, washed with aqueous NaHCO₃, dried (MgSO₄), filtered and concentrated under reduced pressure. The residue was precipitated from ethyl acetate/hexane to provide the titled compound (6.3 g). The filtrate was precipitated a second time to provide and additional 5 g of the titled compound. MS (ESI(+)) m/e 339 (M+H)⁺; ¹H NMR (300 MHz, DMSO-d₆) δ9.31 (s, 1H), 7.40-7.20 (m, 8H), 6.59 (d, 1H), 5.52 (s, 2H), 5.16 (s, 2H), 2.42 (q, 2H), 1.98 (s, 3H), 1.13 (t, 3H).

### Example 79C

### 2-acetylamino-3-(4-amino-3-ethyl-phenyl)-propionic Acid

A mixture of 2-acetylamino-3-(4-amino-3-ethyl-phenyl)-acrylic acid benzyl ester (5 g) and 10% Pd-C (100 mg) in methanol (50 mL) was stirred under an atmosphere of hydrogen (4 atmospheres) at ambient temperature overnight to provide the titled compound. MS (ESI(+)) m/e 251 (M+H)⁺; ¹H NMR (300 MHz, DMSO-d₆) δ8.02 (d, 1H), 6.77-6.70 (m, 2H), 6.50 (d, 1H), 4.31-4.21 (m, 1H), 2.84 (dd, 1H), 2.65 (dd, 1H), 2.39 (q, 2H), 1.78 (s, 3H), 1.10 (t, 3H).

### Example 79D

### 2-acetylamino-3-(4-amino-3-ethyl-phenyl)-propionic Acid Allyl Ester

A mixture of 2-acetylamino-3-(4-amino-3-ethyl-phenyl)-propionic acid (2.0 g, 8.0 mmol), Cs₂CO₃ (2.61 g, 8.0 mmol) and allyl bromide (692 ul, 8.0 mmol) in N,N-dimethylformamide (40 mL) was stirred at room temperature for 3 hours, concentrated under reduce pressure and partitioned between ethyl acetate and water (100 mL, 1:1). The organic phase was washed with brine (1×50 mL), dried (MgSO₄), filtered and concentrated under reduced pressure. The residue was purified by on silica gel with ethyl acetate/hexane (5:3) to provide titled compound (1.44 g). MS (ESI(+)) m/e 291 (M+H)⁺; ¹H NMR (300 MHz, DMSO-d₆) δ8.23 (d, 1H), 6.77-6.70 (m, 2H), 6.50 (d, 1H), 5.90-5.76 (m, 1H), 5.30-5.15 (m, 2H), 4.67 (s, 2H), 4.54-4.50 (m, 2H), 4.38-4.30 (m, 1H), 2.77 (dddd, 2H), 2.39 (q, 2H), 1.80 (s, 3H), 1.10 (t, 3H).

### Example 79E

### 2-{[4-(2-acetylamino-2-allyloxycarbonyl-ethyl)-2-ethyl-phenyl]-tert-butoxyoxlyl-amino}-benzoic Acid

The titled compound was prepared according to the method described in Example 50E by substituting 2-acetylamino-3-(4-amino-3-ethyl-phenyl)-propionic acid allyl ester for 2-{[4-(2-allyloxycarbonylamino-2-pentylcarbamoyl-ethyl)-phenyl]-tert-butoxyoxalylamino}-benzoic acid. MS (APCI (+)) m/e 539 (M+H)⁺.

### Example 79F

### 2-{[4-(2-acetylamino-2-allyloxycarbonyl-ethyl)-2-ethyl-phenyl]-tert-butoxyoxalyl-amino}-benzoic Acid Benzhydryl Ester

To 2-{[4-(2-acetylamino-2-allyloxycarbonyl-ethyl)-2-ethyl-phenyl]-tert-butoxyoxalyl-amino}-benzoic acid in acetone was added diphenyldiazomethane (until all starting material was consumed as evident by monitoring via TLC). The reaction mixture was concentrated under reduced pressure, purified on silica gel using ethyl acetate as eluent to provide the titled compound. MS (ESI(+)) m/e 705 (M+H)⁺; ¹H NMR (300 MHz, DMSO-d₆) δ8.51-8.01 (m, 2H), 7.73-6.86 (m, 16H), 5.93-5.78 (m, 1H), 5.34-5.10 (m, 2H), 4.57-4.40 (m, 3H), 3.10-2.84 (m, 2H), 2.58-2.42 (m, 2H), 1.82-1.77 (m, 3H), 1.22-0.78 (m, 3H), 1.07, 1.05, 1.00 (s. s, s, 9H).

### Example 79G

### (2-{[4-(2-acetylamino-2-carboxy-ethyl)-2-ethyl-phenyl]-tert-butoxyoxalyl-amino}-benzoic Acid Benzhydryl Ester

A mixture of 2-{[4-(2-acetylamino-2-allyloxycarbonyl-ethyl)-2-ethyl-phenyl]-tert-butoxyoxalyl-amino}-benzoic acid benzhydryl ester (3.4 g, 4.8 mmol), Pd(Ph₃P)₄ (166 mg, 0.144 mmol) and morpholine (0.5 ml, 5.8 mmol) in dichloromethane (25 mL) was stirred under N₂ atmosphere for 2 hours, partitioned between ethyl acetate and water (75 mL, 1:1). The organic phase was washed with 1N HCl (1×25 mL), brine (1×25 mL), dried (MgSO₄), filtered and concentrated under reduced pressure to provide the titled compound (3.3 g). MS (ESI(+)) m/e 665 (M+H)⁺; ¹H NMR (300 MHz, DMSO-d₆) δ12.67 (s, 1H), 8.51-7.98 (m, 2H), 7.73-6.86 (m, 16H), 4.53-4.33 (m, 1H), 3.12-2.76 (m, 2H), 2.58-2.42 (m, 2H), 1.82-1.77 (m, 3H), 1.22-0.78 (m, 3H), 1.06, 1.04, 1.00 (s, s, s, 9H).

### Example 79H

### 2-{[4-(2-acetylamino-2-(4-nitro-benzylcarbamoyl)-ethyl]-2-ethyl-phenyl]-tert-butoxyoxalyl-amino}-benzoic Acid Benzhydryl Ester

A mixture of 2-{[4-(2-acetylamino-2-carboxy-ethyl)-2-ethyl-phenyl]-tert-butoxyoxalyl-amino}-benzoic acid benzhydryl ester (25 mg, 0.038 mmol), 4-nitrobenzylamine hydrochloride (15 mg, 0.08 mmol), 2-(1H-benzotriazole-1-yl)-1,1,3,3-tetramethyluronium tetrafluoroborate (16 mg, 0.048 mmol) and diisopropylethylamine (26 μL) in N,N-dimethylformamide (250 μL) was stirred at ambient temperature overnight, concentrated under reduced pressure and the residue purified by reverse-phase HPLC eluting with 5-100% acetonitrile/aqueous 0.1% TRIFLUOROACETIC ACID to provide the titled compound.

### Example 791

### N-acetyl-4-[(carboxycarbonyl)(2-carboxyphenyl)amino]-3-ethyl-N-(4-nitrobenzyl)phenylalaninamide

The material from Example 79H was treated with trifluoroacetic acid/dichloromethane (10 mL, 1:1) at ambient temperature for 3 hours, concentrated under reduced pressure and purified by HPLC eluting with 5-100% acetonitrile/ aqueous 0.1% trifluoroacetic acid to provide the titled compound (8 mg). MS (ESI(+)) m/e 577 (M+H)⁺; ¹H NMR (500 MHz, DMSO-d₆) δ8.65 (t, 1H), 8.24-8.10 (m, 3H), 7.96-7.81 (m, 1H), 7.56-6.75 (m, 8H), 4.59-4.50 (m, 1H), 4.45-4.32 (m, 2H), 3.10-2.97 (m, 1H), 2.88-2.78 (m, 1H), 2.72-2.55 (m, 2H), 1.81, 1.79 (s, s, 3H), 1.22, 0.94 (t, t, 3H).

### EXAMPLE 80

### N-acetyl-4-[(carboxycarbonyl)(2-carboxyphenyl)amino]-3-ethyl-N-[(1R)-1-(4-nitrophenyl)ethyl]phenylalaninamide

The titled compound was prepared according to the procedure described in Example 79 H-I by substituting 1-(4-nitro-phenyl)-ethylamine for 4-nitrobenzylamine in Example 79 H. MS (ESI(+)) m/e 591 (M+H)⁺; ¹H NMR (500 MHz, DMSO-d₆) δ8.68-8.46 (m, 1H), 8.20-8.01 (m, 3H), 7.96-7.81 (m, 1H), 7.59-6.75 (m, 8H), 5.02-4.88 (m, 1H), 4.65-4.50 (m, 1H), 3.00-2.78 (m, 4H), 1.83-1.75 (m, 3H), 1.40-0.86 (m, 6H).

### EXAMPLE 81

### N-acetyl-4-[(carboxycarbonyl)(2-carboxyphenyl)amino]-N-(4-chlorobenzyl)-3-ethylphenylalaninamide

The desired product was prepared according to the procedure described in Example 79 H-I by substituting 4-chlorobenzylamine for 4-nitrobenzylamine in Example 79 H. MS (ESI(+)) m/e 566, 567 (M+H)⁺; ¹H NMR (500 MHz, DMSO-d₆) δ8.51 (t, 1H), 8.20-8.12 (m, 1H), 7.96-7.80 (m, 1H), 7.58-6.75 (m, 10H), 4.57-4.48 (m, 1H), 4.30-4.16 (m, 2H), 3.10-2.97 (m, 1H), 2.88-2.78 (m, 1H), 2.72-2.55 (m, 2H), 1.79, 1.77 (s, s, 3H), 1.22, 0.93(t, t, 3H).

### EXAMPLE 82

### N-acetyl-4-[(carboxycarbonyl)(2-carboxyphenyl)amino]-N-(4-nitrobenzyl)naphthylalaninamide

The titled compound was prepared according to the procedure described in Example 35 D-G, substituting 4-nitro-benzylamine for the amylamine used in Example 35 D. MS (ESI+) m/e 599 (M+H)⁺; ¹H NMR (300 MHz, DMSO-d₆) (A mixture of rotamers) δ8.77-8.60 (m, 1H), 8.49-7.77 (m, 5H), 7.77-7.25 (m, SH), 6.97-6.68 (m, 2H), 4.81-4.60 (m, 2H), 4.43-4.29 (m, 2H), 3.71-3.13 (overlapping m, 5H), 1.88-1.72 (m, 3H).

### EXAMPLE 83

### N-acetyl-4-[(carboxycarbonyl)(2-carboxyphenyl)amino]-N-[(1R)-1-(4-bromophenyl)ethyl]-3-(2-hydroxyethyl)phenylalaninamide

The titled compound was prepared according to the procedure described in Example 35D-G, by substituting 2-acetylamino-3-[4-amino-3-(2-hydroxy-ethyl)-phenyl]-propionic acid for 2-acetylamino-3-(4-amino-naphthalen-1-yl)-propionic acid and 4-bromo-(R)-α-methylbenzylamine for the amylamine used in Example 35D. MS (ESI+) m/e 640, 642 (M+H)⁺; ¹H NMR (300 MHz, DMSO-d₆) (A mixture of rotamers and 1:1 mixture of diastereoisomers) δ8.55-8.27 (m, 2H), 8.20-7.80 (m, 3H), 7.58-6.95 (m, 7H), 6.83-6.64 (m, 1H), 4.93-4.70 (m, 2H), 4.65-4.24 (m, 3H), 3.09-2.63(m, 4H), 1.84-1.72 (multiple s, 3H in total), 1.38-1.05 (m, 3H).

### EXAMPLE 84

### 4-[(butylamino)carbonyl]-2-[(carboxycarbonyl)(7-hydroxy-1-naphthyl)amino]benzoic Acid

The titled compound was prepared according to the procedure described in Example 72I-J substituting n-butylamine for the aqueous ammonia in Example 72I. The titled compound was purified on silica gel eluting with 40% ethyl acetate: hexanes. ¹H NMR (500 MHz, d₆-DMSO) (A mixture of rotamers, major rotamer resonances only) δ9.90 (bs, 1H), 8.51 (t, 1H, J=5.6), 7.94 (d, 1H, J8.1), 7.89 (d, 1H, J=8.1), 7.84 (d, 1H, J=9.1), 7.77 (dd, 1H, J=1.6, 8.1), 7.60 (d, 1H, J=6.6), 7.50 (d, 1H, J=2.2), 7.36 (d, 1H, J=8.1), 7.34 (s, 1H), 7.12 (dd, 1H, J=2.3, 8.9), 3.17-3.10 (m, 2H), 1.45-1.37 (m, 2H), 1.28-1.18 (m, 2H), 0.83 (t, 3H, J=7.3 Hz); MS (ESI)⁻m/z=449 (M−H).

### EXAMPLE 85

### N-acetyl-4-[(carboxycarbonyl)(2-carboxyphenyl)amino]-N-{5 -[(3-hydroxyphenyl)amino]-5-oxopentyl}-3-(1-piperidinyl)phenylalaninamide

### Example 85A

### 2-({4-[2-acetylamino-2-(4-carboxy-butylcarbamoyl)-ethyl]-2-piperidin-1-yl-phenyl}-tert-butoxyoxalyl-amino)-benzoic Acid Benzhydryl Ester

The titled compound was prepared according to the procedure described in Example 55B-J, substituting the 4-bromo-2-piperidin-1-yl-aniline for the 4-bromo-2-ethyl-aniline in Example 55B.

### Example 85B

### N-acetyl-4-[(carboxycarbonyl)(2-carboxyphenyl)amino]-N-{5-[(3-hydroxyphenyl)amino]-5-oxopentyl}-3-(1-piperidinyl)phenylalaninamide

The titled compound was prepared by following the procedure as described in Example 55K, substituting 2-({4-[2-acetylamino-2-(4-carboxy-butylcarbamoyl)-ethyl]-2-piperidin-1-yl-phenyl}-tert-butoxyoxalyl-amino)-benzoic acid benzhydryl ester for 2-({4-[2-acetylamino-2-(4-carboxy-butylcarbamoyl)-ethyl]-2-ethyl-phenyl}-tert-butoxyoxalyl-amino)-benzoic acid benzhydryl ester, and 3-hydroxyaniline for the 1-phenyl-ethylamine used in Example 55K. MS (ESI+) m/e 688 (M+H)⁺; ¹H NMR (300 MHz, DMSO-d₆) (A mixture of rotamers) δ9.79 (s, 1H), 9.75 (s, 1H), 8.06-6.40 (m, 11H), 4.43-4.37 (m, 1H), 3.16 (d, 2H), 3.10-2.9 (m, 6H), 2.02 (s, 3H), 1.6-1.4 (m, 6H).

### EXAMPLE 86

### 2-((carboxycarbonyl){4-[2-hydroxy-3-(pentylamino)propyl]phenyl}amino)benzoic Acid

### Example 86 A

### 1-allyl-4-nitro-benzene

The titled compound was prepared according to the procedure described in JOC, 22, p. 1418, 1957.

### Example 86B

### 4-allyl-phenylamine

A solution of 1-allyl-4-nitro-benzene (1.92 g, 11.8 mmol), NH₄Cl (1.89 g, 35.3 mmol) and iron powder (4.60 g, 82.4 mmol) in 3:1 ethanol:H₂O (200 mL) was heated to reflux overnight. The reaction was cooled, concentrated under reduced pressure and purified on silica gel eluting with ethyl acetate to provide the titled compound. MS (ESI(+)) m/e 134 (M+H)⁺

### Example 86C

### 2-(4-allyl-phenylamino)-benzoic Acid

The titled compound was prepared according to the procedure described in Example 50C substituting 4-allyl-phenylamine for [2-(4-amino-phenyl)-1-pentylcarbamoyl-ethyl]-carbamic acid tert-butyl ester. MS (ESI(+)) m/e 254 (M+H)⁺; ¹H NMR (300 MHz, DMSO-d₆) δ13.02 (bs, 1H), 9.58 (bs, 1H), 7.89 (dd, 1H), 7.39-7.34 (m, 1H), 7.19-7.16 (m, 5H), 6.78-6.73 (m, 1H), 6.03-5.90 (m, 1H), 5.13-5.04 (m, 2H), 3.37-3.33 (m, 2H).

### Example 86D

### 2-[(4-allyl-phenyl)-benzyloxyoxalyl-amino]-benzoic Acid

The titled compound was prepared according to the procedure described in 50E by substituting 2-(4-allyl-phenylamino)-benzoic acid for 2-[4-(2-allyloxycarbonylamino-2-pentylcarbamoyl-ethyl)-phenylamino]-benzoic acid and by substituting benzyl oxalylchloride for t-butyloxalyl chloride. The material was purified on silica gel eluting with methylene chloride, 1% methanol/methylene chloride). The material was obtained as a 1:1 mixture of rotamers. MS (ESI(−)) m/e 414 (M−H)⁺; ¹H NMR (300 MHz, CDCl₃) δ8.07 and 7.92 (2dd, 1H total), 7.57-6.98 (m, 13H), 5.97-5.73 (m, 1H), 5.32 (d, 1H), 5.10-5.02 (m, 2H), 5.02 (d, 1H), 3.35-3.30 (m, 2H).

### Example 86E

### 2-[(4-allyl-phenyl)-benzyloxyoxalyl-amino]-benzoic Acid Benzhydryl Ester

The titled compound was prepared according to the procedure described in Example 50F by substituting 2-[(4-allyl-phenyl)-benzyloxyoxalyl-amino]-benzoic acid for 2-{[4-(2-allyloxycarbonylamino-2-pentylcarbamoyl-ethyl)-phenyl]-tert-butoxyoxalylamino}-benzoic acid. The material was obtained as a 1:1 mixture of rotamers. MS (ESI(−)) m/e 581 (M−H)⁺; ¹H NMR (300 MHz, DMSO-d₆) δ8.10 and 8.06 (2dd, 1H total), 7.72-6.89 (m, 23H), 5.97-5.83 (m, 1H), 5.12-4.85 (m, 4H), 3.28-3.13 (m, 2H).

### Example 86F

### 2-[benzyloxyoxalyl-(4-oxiranylmethyl-phenyl)-amino]-benzoic Acid Benzhydryl Ester

A solution of 2-[(4-allyl-phenyl)-benzyloxyoxalyl-amino]-benzoic acid benzhydryl ester (1.42 g, 2.44 mmol) ) in methylene chloride (15 mL) was treated with 65% meta chloro peroxybenzoic acid (780 mg, 2.94 mmol) and stirred overnight. Chromatography of the reaction mixture (25%-40% EtOAc/hexanes) gave the titled compound as a 1:1 mixture of rotamers. ¹H NMR (300 MHz, DMSO-d₆) δ8.12 and 8.06 (2dd, 1H total), 7.93-7.87 (m, 1H), 7.73-6.89 (m, 22H), 5.03 (s, 1H), 4.96 and 4.84 (2d, 1H total), 3.10-3.03 (m, 1H), 2.78-2.68 (m, 4H).

### Example 86G

### 2-{benzyloxyoxalyl-[4-(2-hydroxy-3-pentylamino-propyl)-phenyl]-amino}-benzoic Acid Benzhydryl Ester

A solution of 2-[benzyloxyoxalyl-(4-oxiranylmethyl-phenyl)-amino]-benzoic acid benzhydryl ester (256 mg, 0.428 mmol) and diisopropylethylamine (112 μL, 0.642 mmol) in 2-methyl-2-propanol (5 mL) was treated with amylamine (55 μL, 0.47 mmol) and refluxed overnight. The reaction was concentrated under reduced pressure and purified by reverse phase HPLC using 20% to 100% acetonitrile/aqueous 0.1% trifluoroacetic acid to provide the titled compound as a 1:1 mixture of rotamers. MS (ESI(+)) m/e 685 (M+H)⁺; ¹H NMR (300 MHz, DMSO-d₆) δ8.10 and 8.05 (2dd, 1H total), 7.71-6.88 (m, 23H), 4.98 (s, 1H), 4.96 and 4.84 (2d, 1H total), 3.68-3.64 (m, 1H), 2.73-2.86 (m, 6H), 1.38-1.33 (m, 2H), 1.26-1.22 (m, 4H), 0.86-0.81 (m, 3H).

### Example 86H

### 2-{[4-(2-Hydroxy-3-pentylamino-propyl)-phenyl]-oxalyl-amino}-benzoic Acid

A solution of 2-{benzyloxyoxalyl-[4-(2-hydroxy-3-pentylamino-propyl)-phenyl]-amino}-benzoic acid benzhydryl ester (60 mg, 0.93 mmol) and 10% Pd/C (100 mg) in methanol (3 mL) was stirred under an atmosphere of hydrogen for 16 hours. The mixture was filtered and the filtrate was concentrated under reduced pressure and purified by reverse phase HPLC with 0% to 70% acetonitrile/ aqueous 0.1% trifluoracetic acid to provide the titled product. MS (ESI(+)) m/e 429 (M+H)⁺; ¹H NMR (300 MHz, DMSO-d₆) δ8.30 (bs, 1H), 7.88-7.39 (m, 5H), 7.29-7.19 (m, 3H), 5.53 (bs, 1H), 3.93 (bs, 1H), 2.94-2.66 (m, 6H), 1.61-1.53 (m, 2H), 1.28-1.23 (m, 4H), 0.89-0.83 (m, 3H).

### EXAMPLE 87

### 2-((carboxycarbonyl){4-[3-(pentylamino)butyl]-1-naphthyl}amino)benzoic Acid

### Example 87A

### 2-[(4-bromo-naphthalen-1-yl)-tert-butoxyoxalyl-amino]-benzoic Acid

The titled compound was prepared according to the procedure described in Example 50C and 50E respectively, by substituting 4-bromo-naphthalen-1-yl-amine for [2-(4-amino-phenyl)-1-pentylcarbamoyl-ethyl]-carbamic acid tert-butyl ester in procedure 50C followed by substituting the product for 2-[4-(2-allyloxycarbonylamino-2-pentylcarbamoyl-ethyl)-phenylamino]-benzoic acid in procedure 50E.

### Example 87B

### 2-[(4-bromo-naphthalen-1-yl)-tert-butoxyoxalyl-amino]-benzoic Acid Benzhydryl Ester

The titled compound was prepared according to the procedure described in Example 50F, substituting 2-[(4-bromo-naphthalen-1-yl)-tert-butoxyoxalyl-amino]-benzoic acid for 2-{[4-(2-allyloxycarbonylamino-2-pentylcarbamoyl-ethyl)-phenyl]-tert-butoxyoxalylamino}-benzoic acid. MS (ESI(+)) m/e 653, 655 (M+NH₄)⁺.

### Example 87 C

### 2-{tert-butoxyoxalyl-[4-(3-oxo-butyl)-naphthalen-1-yl]-amino}-benzoic Acid Benzhydryl Ester

To a mixture of 2-[(4-bromo-naphthalen-1-yl)-tert-butoxyoxalyl-amino]-benzoic acid benzhydryl ester (230 mg, 0.36 mmol), Pd(OAc)₂ (4.0 mg, 0.018 mmol), P(o-tolyl)₃ (11 mg, 0.036 mmol) in anhydrous N,N-dimethylformamide (1.5 mL) in a pressure tube was added 3-buten-2-ol (47 μL, 0.54 mmol) and triethylamine (127 μL, 0.90 mmol). The mixture was flushed with nitrogen for 3 minutes, capped and heated to 100° C. for 30 min. The reaction mixture was allowed to cool to ambient temperature, partitioned between ethyl acetate and water (75 mL, 1:1). The organic layer was washed with brine (2×25 mL), dried (Na₂SO₄), filtered, concentrated under reduced pressure and purified on an Alltech Sep-Pak eluting with 20-30% ethyl acetate/hexanes to provide the titled compound (180 mg, 81%). MS (ESI(+)) m/e 645 (M+NH₄)⁺.

### Example 87 D

### 2-((carboxycarbonyl){4-[3-(pentylamino)butyl]-1-naphthyl}amino)benzoic Acid

A mixture of 2-{tert-butoxyoxalyl-[4-(3-oxo-butyl)-naphthalen-1-yl]-amino}-benzoic acid benzhydryl ester (45 mg, 0.072 mmol) and amylamine (13 μL, 0.11 mmol) in anhydrous methanol (1.0 mL) was stirred at ambient temperature for 3 hours, NaBH₄ (30 mg) was added in portions over 30 minutes, stirred for 2 hours and concentrated under reduced pressure. The residue was dissolved in methylene chloride (1.0 mL) and stirred for 5 hours with trifluoroacetic acid (1.0 mL) and resorcinol (30 mg) then concentrated under reduced pressure. The residue was purified on a Gilson preparative HPLC to provide (22 mg, 0.046 mmol, 63%). MS (ESI+) m/e 477 (M+H)⁺; ¹H NMR (300 MHz, DMSO-d₆) (A mixture of rotamers) δ8.40-8.08 (m, 3H), 7.77-7.25 (m, 6H), 6.70-6.89 (m, 1H), 3.24-2.98 (m, 3H), 2.98-2.80 (m, 2H), 2.20-2.00 (m, 1H), 1.92-1.70 (m, 1H), 1.55 (sixtet, J=7.2 Hz, 2H), 1.37 (t, J=6.9 Hz, 3H), 1.33-1.20 (m, 4H), 0.92-0.82 (m, 3H).

### EXAMPLE 88

### 2-((carboxycarbonyl){4-[3-(pentylamino)propyl]-1-naphthyl}amino)benzoic Acid

The titled compound was prepared according to the procedure described in Example 87, substituting 3-buten-2-ol used in Example 87B with allyl alcohol. MS (ESI+) m/e 640, 642 (M+H)⁺; ¹H NMR (300 MHz, DMSO-d₆) (A mixture of rotamers) δ8.50-8.10 (m, 3H), 7.80-7.27 (m, 6H), 6.90-6.77 (m, 1H), 3.24-2.80 (m, 6H), 2.07-1.90 (m, 2H), 1.63-1.47 (m, 2H), 1.35-1.20 (m, 4H), 0.92-0.82 (m, 3H).

What is claimed is:

1. A compound of formula (I) 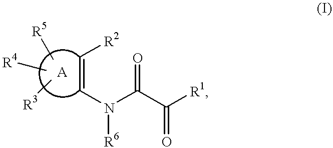
or a therapeutically acceptable salt thereof, wherein
A is selected from the group consisting of aryl, heteroaryl, and heterocycloalkyl;
R¹ is selected from the group consisting of alkoxy, alkyl, amino, aminosulfonyl, aryl, arylalkyl, aryloxy, hydroxy, perfluoroalkoxy, and perfluoroalkyl;
R² is selected from the group consisting of alkoxy, alkoxycarbonyl, alkyl, amido, amino, carboxy, cyano, nitro, SO₃H, PO(OH)₂, CH₂PO(OH)₂, CHFPO(OH)₂, CF₂(PO(OH)₂, c(═NH)NH₂, and the following 5-membered heterocycles: 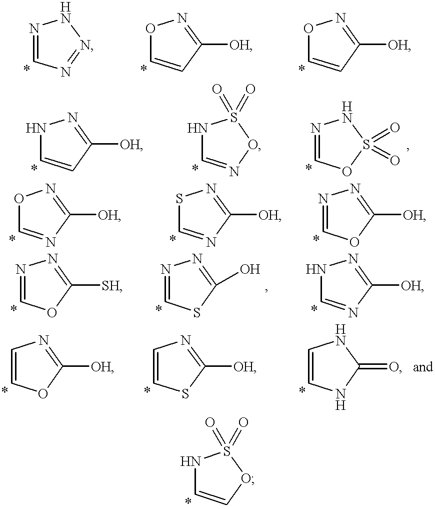
wherein * denotes the point of attachment to the parent molecular moeity;
R³, R⁴, and R⁵ are independently selected from the group consisting of hydrogen, alkoxy, alkyl, amido, amino, aminosulfonyl, arylcarbonylamino, cyano, halo, hydroxy, nitro, perfluoroalkoxy, and perfluoroalkyl; and
R⁶ is selected from the group consisting of alkyl, aryl, arylalkyl, cycloalkenyl, cycloalkenylalkyl, cycloalkyl, cycloalkylalkyl, heteroaryl, heteroarylalkyl, heterocycloalkyl, and (heterocycloalkyl)alkyl.

2. A compound according to claim 1 of formula (II), 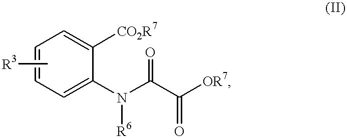
or a therapeutically acceptable salt thereof, wherein
R³ is selected from the group consisting of hydrogen, amido, alkoxy, arylcarbonylamino, cyano, hydroxy,
R⁶ is selected from the group consisting of alkyl, aryl, arylalkyl, cycloalkenyl, cycloalkenylalkyl, cycloalkyl, cycloalkylalkyl, heteroaryl, heteroarylalkyl, heterocycloalkyl, and (heterocycloalkyl)alkyl; and
Each R⁷ is independently selected from the group consisting of hydrogen and alkyl.

3. A compound according to claim 1 wherein A is aryl.

4. A compound according to claim 3 wherein R¹ is hydroxy.

5. A compound according to claim 4 wherein R² is carboxy.

6. A compound according to claim 5 wherein R³, R⁴, and R⁵ are independently selected from the group consisting of hydrogen, alkoxy, amido, arylcarbonylamino, cyano and hydroxy.

7. A compound according to claim 6 wherein R⁶ is aryl.

8. A compound according to claim 6 wherein R⁶ is cycloalkyl.

9. A compound according to claim 6 wherein R⁶ is heteroaryl.

10. A method for inhibiting protein tyrosine phosphatase at physiological pH comprising administering a therapeutically effective amount of a compound of claim 1.

11. A method for inhibiting protein tyrosine phosphatase at about pH 6.5 to about pH 8.5 comprising administering a therapeutically effective amount of a compound of claim 1.

12. The method of claim 11 wherein the pH is about 7.5.

13. A method for treating diseases in a patient in recognized need thereof comprising administering to the patient a therapeutically effective amount of a compound of claim 1.

14. The method of claim 13 wherein the disease is selected from the group consisting of type II diabetes, obesity, impaired glucose tolerance and insulin resistance.

15. A composition comprising a compound of claim 1 in combination with a therapeutically acceptable excipient.

16. A method for inhibiting protein tyrosine phosphatase at physiological pH comprising administering a therapeutically effective amount of a compound of claim 2.

17. A method for inhibiting protein tyrosine phosphatase at about pH 6.5 to about pH 8.5 comprising administering a therapeutically effective amount of a compound of claim 2.

18. The method of claim 17 wherein the pH is about 7.5.

19. A method for treating diseases in a patient in recognized need thereof comprising administering to the patient a therapeutically effective amount of a compound of claim 2.

20. The method of claim 19 wherein the disease is selected from the group consisting of type II diabetes and obesity, impaired glucose tolerance and insulin resistance.

21. A composition comprising a compound of claim 2 in combination with a therapeutically acceptable excipient.

22. A compound selected from the group consisting of
2-((carboxycarbonyl)-2-((E)-2-carboxyethenyl)anilino)benzoic acid;
2-(2-((1E)-3-((tert-butoxycarbonyl)amino)-1-propenyl)(carboxycarbonyl)anilino)benzoic acid;
2-((carboxycarbonyl)-2,3-dimethylanilino)benzoic acid;
2-((carboxycarbonyl)-4-chloro-3-methylanilino)benzoic acid;
2-(2-(aminocarbonyl)(carboxycarbonyl)anilino)benzoic acid;
2-((7-(aminomethyl)-5,6,7,8-tetrahydro-1-naphthalenyl)(carboxycarbonyl)amino)benzoic acid;
2-((6-(aminomethyl)-5,6,7,8-tetrahydro-1-naphthalenyl)(carboxycarbonyl)amino)benzoic acid;
2-((carboxycarbonyl)-4-(2,3-diamino-3-oxopropyl)anilino)benzoic acid; and
2-((carboxycarbonyl)(5-(2,3-diamino-3-oxopropyl)-5,6,7,8-tetrahydro-1-naphthalenyl)amino)benzoic acid.

23. A compound selected from the group consisting of
2-((carboxycarbonyl)(1-naphthyl)amino)benzoic acid;
2-((carboxycarbonyl)(2-naphthyl)amino)benzoic acid;
2-((carboxycarbonyl)-4-methoxyanilino)benzoic acid;
2-((carboxycarbonyl)(1-naphthyl)amino)-5-methoxybenzoic acid;
2-((carboxycarbonyl)-2-chloro-5-methoxyanilino)benzoic acid;
2-((carboxycarbonyl)-2-((1E)-3-ethoxy-3-oxo-1-propenyl)anilino)benzoic acid;
2-(4-((2S)-2-((tert-butoxycarbonyl)amino)-3-(((4-(methoxycarbonyl)cyclohexyl)methyl)amino)-3-oxopropyl)(carboxycarbonyl)anilino)benzoic acid;
2-(4-((2S)-2-((tert-butoxycarbonyl)amino)-3-(((4-carboxycyclohexyl)methyl)amino)-3-oxopropyl)(carboxycarbonyl)anilino)benzoic acid;
2-((carboxycarbonyl)-2-iodoanilino)benzoic acid;
2-((carboxycarbonyl)-2-((1E)-3-(1,3-oxazinan-3-yl)-3-oxo-1-propenyl)anilino)benzoic acid;
2-((carboxycarbonyl)-3-(trifluoromethyl)anilino)benzoic acid;
2-((carboxycarbonyl)(cyclobutyl)amino)benzoic acid;
2-((7-(benzyloxy)-1-naphthyl)(carboxycarbonyl)amino)benzoic acid;
2-((carboxycarbonyl)-2-(2-hydroxyethyl)anilino)benzoic acid;
2-((carboxycarbonyl)-2-methylanilino)benzoic acid;
2-((carboxycarbonyl)(2-methyl-1H-indol-1-yl)amino)benzoic acid;
2-((carboxycarbonyl)(7-hydroxy-1-naphthyl)amino)benzoic acid;
2-((carboxycarbonyl)(7-((6-phenylhexyl)oxy)-1-naphthyl)amino)benzoic acid;
2-((1,1′-biphenyl)-2-yl(carboxycarbonyl)amino)benzoic acid;
2-((1,1′-biphenyl)-4-yl(carboxycarbonyl)amino)benzoic acid;
2-((carboxycarbonyl)(5,6,7,8-tetrahydro-1-naphthalenyl)amino)benzoic acid;
2-((carboxycarbonyl)(cyclohexyl)amino)benzoic acid;
2-((carboxycarbonyl)(2,3-dihydro-1,4-benzodioxin-6-yl)amino)benzoic acid;
2-((carboxycarbonyl)(3-methylcyclohexyl)amino)benzoic acid;
2-[(carboxycarbonyl)(2-hydroxy-1-naphthyl)amino]benzoic acid;
N-acetyl-4-[(carboxycarbonyl)(2-carboxyphenyl)amino]-N-pentyl-1-naphthylalaninamide;
N-acetyl-4-[(carboxycarbonyl)(2-carboxyphenyl)amino]-N-pentyl-3-(2-hydroxyethane)-phenylalaninamide;
4-[(carboxycarbonyl)(2-carboxyphenyl)amino]-6-{[N-acetyl-3-(1-naphthyl)alanyl]amino}hexanoic acid;
4-[(carboxycarbonyl)(2-carboxyphenyl)amino]-3-[(1E)-3-amino-3-oxo-1-propenyl]-N-(tert-butoxycarbonyl)-N-pentyl-L-phenylalaninamide;
N-acetyl-4-[(carboxycarbonyl)(2-carboxyphenyl)amino]-3-isopropyl-N-pentylphenylalaninamide;
4-[(carboxycarbonyl)(2-carboxyphenyl)amino]-6-{[N-acetyl-3-(1-piperidinyl)phenylalanyl]amino}hexanoic acid;
2-{(carboxycarbonyl)[2-(3-methyl-1-piperidinyl)phenyl]amino}benzoic acid;
2-{(carboxycarbonyl)[5-hydroxy-2-(1-piperidinyl)phenyl]amino}benzoic acid;
4-[(carboxycarbonyl)(2-carboxyphenyl)amino]-3-[(1E)-3-amino-3-oxo-1-propenyl]-N-(methylsulfonyl)-N-pentyl-L-phenylalaninamide;
4-[(carboxycarbonyl)(2-carboxyphenyl)amino]-3-(3-amino-3-oxopropyl)-N-[(isopropylamino)carbonyl]-N-pentyl-L-phenylalaninamide;
4-[(carboxycarbonyl)(2-carboxyphenyl)amino]-3-[(1E)-3-amino-3-oxo-1-propenyl]-N-[(isopropylamino)carbonyl]-N-pentyl-L-phenylalaninamide;
2-((carboxycarbonyl){2-[4-(hydroxymethyl)-1-piperidinyl]phenyl}amino)benzoic acid;
N-acetyl-4-[(carboxycarbonyl)(2-carboxyphenyl)amino]-N-pentyl-4-(1-piperidinyl)phenylalaninamide;
N-acetyl-4-[(carboxycarbonyl)(2-carboxyphenyl)amino]-3-ethylphenylalanyl-N-methyl-4-nitro-L-phenylalaninamide;
N-(3-carboxypropanoyl)-L-phenylalanyl-3-[(1E)-3-amino-3-oxo-1-propenyl]-4-[(carboxycarbonyl)(2-carboxyphenyl)amino]-N-pentyl-L-phenylalaninamide;
3-(4-benzoylphenyl)-N-(tert-butoxycarbonyl)-L-alanyl-3-{4-[(carboxycarbonyl)(2-carboxyphenyl)amino]phenyl}-N˜1˜-pentyl-L-alaninamide;
N-acetyl-4-[(carboxycarbonyl)(2-carboxyphenyl)amino]-3-(2-hydroxyethyl)-N-[4-(methylsulfonyl)benzyl]phenylalaninamide;
2-[[7-(aminocarbonyl)-1-naphthyl](carboxycarbonyl)amino]benzoic acid;
N-acetyl-4-[(carboxycarbonyl)(2-carboxyphenyl)amino]-3-isopropyl-N-[4-(methylsulfonyl)benzyl]phenylalaninamide
4-[(carboxycarbonyl)(2-carboxyphenyl)amino]-1-acetyl-6-(3-isopropylbenzyl)-4-[4-(methylsulfonyl)benzyl]-2,3,5-piperazinetrione;
2-[(carboxycarbonyl)(7-hydroxy-1-naphthyl)amino]-4-hydroxybenzoic acid;
N-acetyl-4-[(carboxycarbonyl)(2-carboxyphenyl)amino]-3-ethyl-N-{5-oxo-5-[(1-phenylethyl)amino]pentyl}phenylalaninamide;
N-(methoxycarbonyl)-4-[(carboxycarbonyl)(2-carboxyphenyl)amino]-N-pentylnaphthylalaninamide;
4-[(carboxycarbonyl)(2-carboxyphenyl)amino]-N-(cyclohexylmethyl)-N-(methoxycarbonyl)naphthylalaninamide;
4-[(carboxycarbonyl)(2-carboxyphenyl)amino]-N-(methoxycarbonyl)-N-[(1R)-1-(4-nitrophenyl)ethyl]naphthylalaninamide;
4-[(carboxycarbonyl)(2-carboxyphenyl)amino]-N-(methoxycarbonyl)-N-[4-(methylsulfonyl)benzyl]naphthylalaninamide;
4-[(carboxycarbonyl)(2-carboxyphenyl)amino]-N-(methoxycarbonyl)-N-(3,4,5-trifluorobenzyl)naphthylalaninamide;
4-[(carboxycarbonyl)(2-carboxyphenyl)amino]-N-(cyclooctylmethyl)-N-(methoxycarbonyl)naphthylalaninamide;
4-[(carboxycarbonyl)(2-carboxyphenyl)amino]-N-[(1R)-1-(4-bromophenyl)ethyl]-N-(methoxycarbonyl)naphthylalaninamide;
4-[(carboxycarbonyl)(2-carboxyphenyl)amino]-N-(methoxycarbonyl)-N-(3-phenylpropyl)naphthylalaninamide;
methyl 3-{4-[(carboxycarbonyl)(2-carboxyphenyl)amino]-1-naphthyl}-N-(methoxycarbonyl)alanyl-L-norleucinate;
4-[(carboxycarbonyl)(2-carboxyphenyl)amino]-N-(2-fluorobenzyl)-N-(methoxycarbonyl)naphthylalaninamide;
4-[(carboxycarbonyl)(2-carboxyphenyl)amino]-N-(4-chlorobenzyl)-N-(methoxycarbonyl)naphthylalaninamide;
4-[(carboxycarbonyl)(2-carboxyphenyl)amino]-N-(4-bromobenzyl)-N-(methoxycarbonyl)naphthylalaninamide;
4-[(carboxycarbonyl)(2-carboxyphenyl)amino]-N-(methoxycarbonyl)-N-(4-nitrobenzyl)naphthylalaninamide;
4-[(carboxycarbonyl)(2-carboxyphenyl)amino]-N-[4-(aminosulfonyl)benzyl]-N-(methoxycarbonyl)naphthylalaninamide;
4-[(carboxycarbonyl)(2-carboxyphenyl)amino]-N-(methoxycarbonyl)-N-({4-[(methylamino)carbonyl]cyclohexyl}methyl)naphthylalaninamide;
N-acetyl-4-[(carboxycarbonyl)(2-carboxyphenyl)amino]-3-(2-hydroxyethyl)-N-(4-nitrobenzyl)phenylalaninamide;
2-[(carboxycarbonyl)(7-hydroxy-1-naphthyl)amino]-4-cyanobenzoic acid;
4-[(carboxycarbonyl)(2-carboxyphenyl)amino]-N-{4-[(ethylamino)sulfonyl]benzyl}-N-(methoxycarbonyl)naphthylalaninamide;
N-(tert-butoxycarbonyl)-L-phenylalanyl-3-[(1E)-3-amino-3-oxo-1-propenyl]-4-[(carboxycarbonyl)(2-carboxyphenyl)amino]-N-pentyl-L-phenylalaninamide;
N-acetyl-4-[(carboxycarbonyl)(2-carboxyphenyl)amino]-3-(2-hydroxyethyl)-N-[(1S)-1-(4-nitrophenyl)ethyl]phenylalaninamide;
N-acetyl-4-[(carboxycarbonyl)(2-carboxyphenyl)amino]-N-(4-chlorobenzyl)-3-(2-hydroxyethyl)phenylalaninamide;
N-acetyl-4-[(carboxycarbonyl)(2-carboxyphenyl)amino]-N-(4-bromobenzyl)-3-(2-hydroxyethyl)phenylalaninamide;
2-[(carboxycarbonyl)(7-hydroxy-1-naphthyl)amino]-4-{[4-(dimethylamino)benzoyl]amino}benzoic acid;
N-acetyl-4-[(carboxycarbonyl)(2-carboxyphenyl)amino]-3-ethyl-N-(4-nitrobenzyl)phenylalaninamide;
N-acetyl-4-[(carboxycarbonyl)(2-carboxyphenyl)amino]-3-ethyl-N-[(1R)-1-(4-nitrophenyl)ethyl]phenylalaninamide;
N-acetyl-4-[(carboxycarbonyl)(2-carboxyphenyl)amino]-N-(4-chlorobenzyl)-3-ethylphenylalaninamide;
N-acetyl-4-[(carboxycarbonyl)(2-carboxyphenyl)amino]-N-(4-nitrobenzyl)naphthylalaninamide;
N-acetyl-4-[(carboxycarbonyl)(2-carboxyphenyl)amino]-N-[(1R)-1-(4-bromophenyl)ethyl]-3-(2-hydroxyethyl)phenylalaninamide;
4-[(butylamino)carbonyl]-2-[(carboxycarbonyl)(7-hydroxy-1-naphthyl)amino]benzoic acid;
N-acetyl-4-[(carboxycarbonyl)(2-carboxyphenyl)amino]-N-{5-[(3-hydroxyphenyl)amino]-5-oxopentyl}-3-(1-piperidinyl)phenylalaninamide;
2-((carboxycarbonyl){4-[2-hydroxy-3-(pentylamino)propyl]phenyl}amino)benzoic acid;
2-((carboxycarbonyl){4-[3-(pentylamino)butyl]-1-naphthyl}amino)benzoic acid; and
2-((carboxycarbonyl){4-[3-(pentylamino)propyl]-1-naphthyl}amino)benzoic acid.
